(19) United States
(12) Patent Application Publication
(10) Pub. No.: US 2025/0277793 A1
(43) Pub. Date: Sep. 4, 2025

(54) COMPOSITIONS AND METHODS FOR CANCER DIAGNOSIS

(71) Applicant: AOA Dx, Cambridge, MA (US)

(72) Inventor: Horacio Uri Saragovi, Montreal (CA)

(21) Appl. No.: 18/030,426

(22) PCT Filed: Oct. 5, 2021
(86) PCT No.: PCT/US21/53503

§ 371 Date: Apr. 5, 2023

Related U.S. Application Data
(60) Provisional application No. 63/087,427, filed on Oct. 5, 2020.

(51) Int. Cl. G01N 33/574; A61K 39/00; C07K 16/30

(52) U.S. Cl. CPC G01N 33/57484; A61K 39/001171; C07K 16/3084; C07K 2317/73; G01N 2405/10; G01N 2560/00

(57) ABSTRACT

The present disclosure provides monoclonal antibodies and antigen-binding fragments thereof that specifically bind to gangliosides (e.g., GD2 or GD3), as well as compositions comprising same and methods of using same for diagnostic and prognostic purposes. In addition, the present disclosure provides mass spectrometry-based methods for detecting various gangliosides and their lipid length, the changes of which are useful for the cancer diagnostic and prognostic methods described herein. The present disclosure further provides compounds comprising modified versions of gangliosides.

### CROSS-REFERENCE TO RELATED APPLICATIONS

This application claims the benefit of U.S. Provisional Application No. 63/087,427, filed Oct. 5, 2020, the entire contents of said application is incorporated herein in its entirety.

### BACKGROUND OF THE INVENTION

Cancer involves abnormal cell growth with the potential to invade or spread to other parts of the body. Despite decades of cancer research, cancer continues to cause a significant number of deaths (˜1,600 deaths per day in the U.S. in 2020) largely due to a lack of means for early and/or accurate detection. For example, ovarian cancer is the most lethal gynecologic cancer and the third cause of death among women. Serous Ovarian Cancer is the most aggresive subtype among ovarian cancer and it has been commonly labelled as the “silent killer” because most patients are diagnosed at advanced stages. Symptoms are vague and easily confounded with other conditions. Currently, CA-125 is a blood marker used both to screen and to monitor treatment efficacy. Although CA-125 levels are raised in 80% of epithelial tumors, most of these tumors are in an advanced stage. Serum CA-125 has a low detection rate for early diagnosis because its levels are elevated in <50% of stage I ovarian cancers, and has limited specificity. Thus, there is a great need for new biomarkers, e.g., in cancer tissue and in blood, to detect early stages of cancer, which would improve the survival of cancer patients.

### SUMMARY OF THE INVENTION

Gangliosides are glycolipids that comprise (i) a carbohydrate structure that specifically defines it by name, and (ii) lipid tails that can vary in carbon chain length. The present invention is based, at least ins part, on the discovery that gangliosides (e.g., GD2, GD3, GM2, and/or GD1) are useful biomarkers for cancer diagnosis and prognosis, especially for early detection of cancer. Provided herein are compositions and methods for cancer diagnosis and prognosis. For example, provided herein are compositions comprising monoclonal antibodies, or an antigen-binding fragment thereof, that specifically bind a ganglioside (e.g., GD2 or GD3), and methods of using same for detecting and measuring the amount of a ganglioside, as well as diagnosing a cancer using a liquid biopsy (e.g., blood, serum) or solid tissue biopsy. Further provided herein are novel mass spectrometry-based methods to detect the presence, level, and/or lipid length of a ganglioside (e.g., GD2, GD3, GM2, and/or GD1). The heterogeneity and/or homogeneity of the type of gangliosides detected, as well as the heterogeneity and/or homogeneity or changes thereof in the lipid length in cancer patients, or longitudinally in a patient over time, can thereby provide a novel biomarker for cancer diagnosis and prognosis.

In certain aspects, provided herein is a composition comprising a modified ganglioside. For example, provided herein is a composition comprising a ganglioside having the structure:

(A)x-[(P)y-(L)z]-(M)b;
wherein A is a ganglioside, or any portion thereof; x is an integer from 1 to 32; P is a heteroaryl; y is 1; L is a linker; z is an integer from 0 to 8; M is a core; and b is 0 or 1;
wherein P is optionally substituted with 1, 2, 3, 4, or 5 substituents independently selected from (1) hydrogen; (2) C₁₋₇ acyl; (3) C₁₋₂₀ alkyl; (4)amino; (5) C₃₋₁₀ aryl; (6) hydroxy; (7) nitro; (8) C₁₋₂₀ alkyl-amino; and (9)-(CH₂)_{q}CONR^{B}, where q is an integer from 0 to 4 and where R^{B} is selected from the group consisting of (a) hydrogen, (b) C₁₋₆ alkyl, (c) C₃₋₁₀ aryl, and (d) C₁₋₆ alk-C₆₋₁₀ aryl.

In some embodiments, the ganglioside comprises a triazine. In other embodiments, the modified ganglioside comprises a triazole.

In some embodiments, the ganglioside of the present disclosure is detectably labeled. In some embodiments, the composition comprising the ganglioside of the present disclosure is a pharmaceutical composition.

Also provided herein is a method of inducing an immune response against a ganglioside in a subject, comprising administering to the subject the composition comprising the ganglioside of the present disclosure.

Further provided herein is a method of treating a subject in need thereof, the method comprising administering to the subject the composition comprising the ganglioside of the present disclosure. In some embodiments, the subject is afflicted with a cancer or has an infection (e.g., viral (e.g., HIV, hepatitis C virus (HCV), or Epstein-Barr virus) or bacterial infection). For example, the composition of the present disclosure can be used to prevent or treat a cancer in a subject.

Provided herein is a method of producing an antibody in a mammal, comprising: (a) immunizing the mammal with the composition comprising the ganglioside of the present disclosure, optionally further comprising an adjuvant; and (b) isolating an antibody that binds to the ganglioside from the mammal, a cell from the mammal, or a hybridoma made using a cell from the mammal. In some embodiments, the mammal is selected from a rabbit, a mouse, a goat, a camel, a dog, a sheep, or a rat.

In certain aspects, provided herein is a monoclonal antibody, or antigen-binding fragment thereof, wherein the monoclonal antibody specifically binds to the carbohydrate portion of a ganglioside. In some embodiments, the ganglioside is (a) GD2, (b) GD3, or (c) GD2 and GD3.

In certain aspects, provided herein is an isolated nucleic acid molecule that encodes: a) a polypeptide comprising an amino acid sequence listed in Table 1 and/or Table 2; b) a polypeptide comprising an amino acid sequence with at least or about 85% identity to an amino acid sequence listed in Table 1 and/or Table 2; and/or c) the monoclonal antibody or antigen-binding fragment thereof, described herein.

In certain aspects, a vector comprising the isolated nucleic acid described herein, is provided.

In certain aspects, a host cell which comprises the isolated nucleic acid described herein, comprises the vector described herein, expresses the antibody, or antigen-binding fragment thereof, described herein, is provided.

In certain aspect, a method of producing at least one monoclonal antibody or antigen-binding fragment thereof, of the present disclosure, wherein the method comprises the steps of: (i) culturing a host cell comprising a nucleic acid comprising a sequence encoding at least one monoclonal antibody, or antigen-binding fragment thereof, of the present disclosure under conditions suitable to allow expression of said monoclonal antibody or antigen-binding fragment thereof; and (ii) recovering the expressed monoclonal antibody or antigen-binding fragment thereof.

In certain aspects, a device or kit comprising at least one monoclonal antibody, or antigen-binding fragment thereof, described herein.

In some embodiments, the device or kit further comprises: (a) a label to detect the at least one monoclonal antibody or antigen-binding fragment thereof; (b) a secondary antibody for detection of the primary antibody; and/or (c) at least one reference antigen, optionally wherein the reference antigen is a ganglioside.

In some embodiments, the reference antigen of the device or kit is selected from GD2, GD3, and a modified version of GD2 or GD3. In some embodiments, the reference antigen is selected from any one of the gangliosides described herein (e.g., those having the structure of (A)x-[(P)y-(L)z]-(M)b), thiophenyl GD2, thiophenyl GD3, GD2-O-aryl-NH2, GD3-O-aryl-NH2, p-amino phenyl ether GD2 (AP-GD2), p-amino phenyl ether GD3 (AP-GD3), triazine GD2 (e.g., 1, 3, 5-triazine-GD2, e.g., 2-Amino-4,6-dichloro-1,3,5-triazine-GD2), triazine GD3 (e.g., 1, 3, 5-triazine-GD3, e.g., 2-Amino-4,6-dichloro-1,3,5-triazine-GD3), a multimeric GD2 (e.g., PAMAM (polyamidoamine)-GD2), and a multimeric GD3 (PAMAM-GD3).

In certain aspects, a method of extracting lipids from a sample, the method comprising: (a) obtaining the sample; (b) adding to the sample about 1, 2, 3, 4, or 5-fold (or 2 to 5-fold) volume of an organic solvent comprising CHCl₃: methanol: water at a ratio selected from (i) about 1:2:1, (ii) about 4:8:3, or (iii) about 2:4:1; (c) shaking the combined mixture of (b); and (d) separating the sample from the organic solvent, thereby extracting lipids from the sample.

In certain aspects, a method of purifying a ganglioside from a sample, the method comprising: (a) obtaining the sample; (b) adding to the sample about 1, 2, 3, 4, or 5-fold (or 2 to 5-fold) volume of an organic solvent comprising CHCl₃: methanol: water at a ratio selected from (i) about 1:2:1, (ii) about 4:8:3, or (iii) about 2:4:1; (c) shaking the combined mixture of (b); and (d) separating the sample from the organic solvent, thereby extracting lipids from the sample.

In some embodiments of the method of extracting lipids or the method of purifying a ganglioside, (i) the sample is clarified by centrifugation prior to extraction with the organic solvent; (ii) the sample is from a mammal, optionally from a human; (iii) the sample is separated from the organic solvent by centrifugation; (iv) the sample is from a subject afflicted with a cancer or a cancer-free subject; (v) the sample comprises cells, serum, blood, peritumoral tissue, and/or intratumoral tissue; (vi) the method further comprises repeating the steps of (b)-(d) at least 1, 2, 3, 4, or 5 times; and/or (vii) the method further comprises evaporating the residual organic solvent from the extracted sample of (d), optionally by centrifuging the solution under vacuum (e.g., speed vacuum).

For samples comprising cells and/or tissues (i.e., non-liquid), an ordinarily skilled artisan appreciates that such samples should be sufficiently homogenized to increase the efficiency of the extraction. Accordingly, in some embodiments, the cells and/or tissues are homogenized before extraction.

In certain aspects, a method of detecting the presence or level of at least one ganglioside (e.g., GD2 and/or GD3) comprising detecting said ganglioside in a sample using at least one monoclonal antibody, or antigen-binding fragment thereof, described herein. In some embodiments, wherein the sample is from a subject afflicted with a cancer or a cancer-free subject.

As described herein, certain embodiments are applicable to any method described herein. For example, in some embodiments, the at least one monoclonal antibody, or antigen-binding fragment thereof, forms a complex with a ganglioside (e.g., GD2 or GD3) and the complex is detected in the form of an enzyme linked immunosorbent assay (ELISA), radioimmune assay (RIA), immunochemically (e.g., immunohistochemistry (IHC)), or using a flow cytometry. In some embodiments, the complex is detected in an enzyme linked immunosorbent assay (ELISA). In preferred embodiments, the complex is detected in sandwich ELISA or competitive ELISA. In some embodiments, the complex is detected by immunohistochemistry (IHC).

In preferred embodiments, the complex is detected in sandwich ELISA. In some embodiments, the complex is detected in sandwich ELISA using any two antibodies or antigen-binding fragment thereof of the present disclosure. In other embodiments, the complex is detected in sandwich ELISA using a combination of any one antibody of the present disclosure and an antibody known in the art (e.g., an antibody that can detect a ganglioside, e.g., an antibody directed to the ceramide or the lipid tail moiety of a ganglioside). In yet other embodiments, the complex is detected in sandwich ELISA using two antibodies or antigen-binding fragment thereof that are known in the art.

In another preferred embodiments, the complex is detected in competitive ELISA.

In some embodiments, the competititve ELISA comprises a reference antigen that is selected from GD2, GD3, or a modified version of GD2 or GD3. In some embodiments, the reference antigen is selected from any one of the gangliosides described herein (e.g., those having the structure of (A)x-[(P)y-(L)z]-(M)b), thiophenyl GD2, thiophenyl GD3, GD2-O-aryl-NH2, GD3-O-aryl-NH2, p-amino phenyl ether GD2 (AP-GD2), p-amino phenyl ether GD3 (AP-GD3), triazine GD2 (e.g., 1, 3, 5-triazine-GD2, e.g., 2-Amino-4,6-dichloro-1,3,5-triazine-GD2), triazine GD3 (e.g., 1, 3, 5-triazine-GD3, e.g., 2-Amino-4,6-dichloro-1,3,5-triazine-GD3), a multimeric GD2 (e.g., PAMAM-GD2), and a multimeric GD3 (PAMAM-GD3).

Further provided herein is a mass spectrometry-based method of detecting at least one ganglioside. For example, provided herein is a method of detecting the presence, the level, or lipid length of at least one ganglioside comprising detecting said ganglioside in a sample using mass spectrometry (e.g., LC/MS or LC/MS/MS).

In some embodiments, the relative heterogeneity or alternatively the relative homogeneity of the lipid length of at least one ganglioside is detected and/or quantified using said method. In some embodiments, the longitudinal changes (changes made over a time for a given patient) that occur in a patient to the relative heterogeneity or relative homogeneity of the lipid length of at least one ganglioside is detected and/or quantified using said method.

In some embodiments, the sample for detecting the presence, level, or the lipid length of at least one ganglioside (e.g., detection using an antibody or mass spectrometry) comprises a sample prepared according to the method described herein (e.g., lipid extraction).

In certain aspects, provided herein are diagnostic and prognostic methods that are described in details herein.

### BRIEF DESCRIPTION OF FIGURES

FIG. 1 shows a schematic diagram of GD2 and GD3 gangliosides. Each geometric shape is a type of sugar. The exposed glycan tree is linked to a ceramide, which is linked to two lipid tails. The sugar head of normal GM1 and the tumor GD3 differ by 2 sugars, and GD2 and GD3 differ from each other by 1 sugar. GD2 is a biosynthetic product of GD3, so the GD2 and GD3 can be often (but not always) detected on the same cell.

FIG. 2 shows the specificity of GD2 and GD3 mAbs. Example of anti-GD2 mAbs 9A3 and 14C₃ in flow cytometry, specifically binding to cells expressing GD2, but not to cells lacking GD2 but expressing GD3. The red histogram is a mAb negative control.

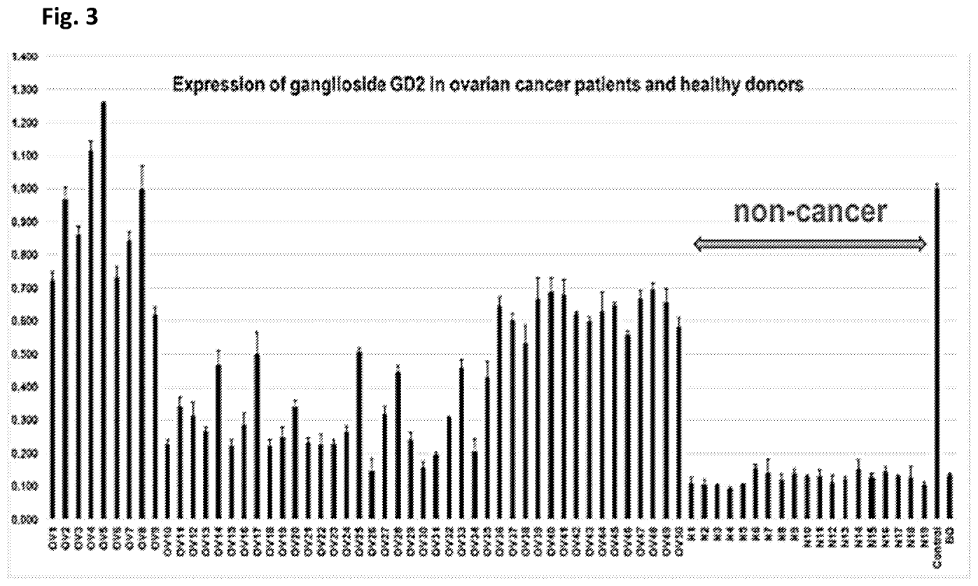

FIG. 3 shows ELISA assays quantify the amounts of GD2 or GD3 in the serum of ovarian cancer patients (representative of multiple assays). Analytes immobilized in ELISA plates are assayed with mAbs to GD2 or GD3. Control=10 ng purified native GD2 or GD3. BG=background wells, no primary. All cancer samples (OV) are positive for GD2 or GD3 except one diagnosed borderline. All normal samples (N) are negative and equal to background. The OD for each sample is average of triplicates±sd, and each ELISA was replicated at least 2 independent times, and two different mAbs each for GD2 or for GD3. As cutoff a statistically significant 2-fold higher value was used and at least 2×s.d from the average of all healthy controls or background. The ODs are normalized to the standard curve.

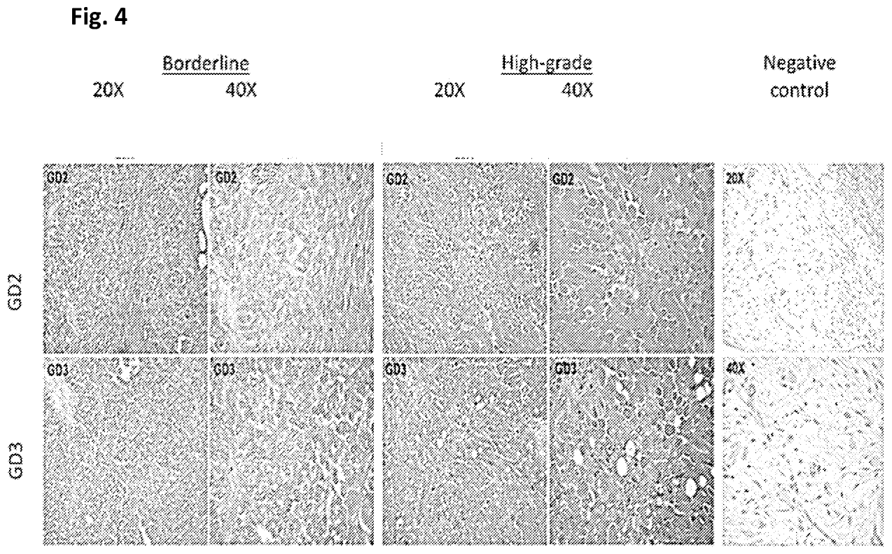

FIG. 4 shows GD2 and GD3 immuno-staining on human ovarian tissues. GD2 and GD3 staining was specifically detected in tumor cells of borderline and high-grade biopsies. Only cancer tissue was GD2+ or GD3+ with robust labeling of the tumor membrane, surrounding normal tissue was negative. Negative control is an isotype matched irrelevant mAb tested on an adjacent section.

FIG. 5 shows that GD2 and GD3 IHC on human ovarian tissue correlates with disease stage. GD3 and/or GD2 staining were detected in tumor tissues, correlating with disease stage (p=0.026 and p=0.015 comparing low grade to high grade, without further segregation by subtype). High grades express both GD2 and GD3 and scored high in expression of GD2 and/or GD3. Low grades scored lower for both GD2 and GD3 expression, albeit GD2 stained slightly stronger than GD3.

FIG. 6 shows immunostaining of GD2 and GD3 in ovarian cancer tissue biopsies. Both borderline (early stage) and high grade (late stage) biopsies are positive for GD2 and GD3 in IHC. The liquid biopsy data correlate with IHC when samples from the same patient is studied using the 2 techniques.

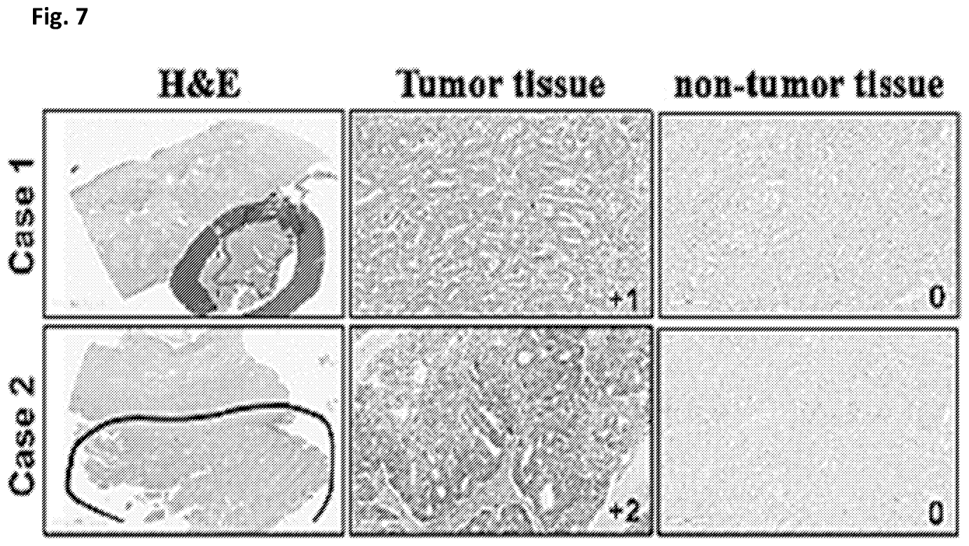

FIG. 7 shows immunostaining of GD2 and GD3 in melanoma tissue biopsies. Nearly 100% of the melanoma tissue biopsies are positive for GD2 and GD3 in IHC.

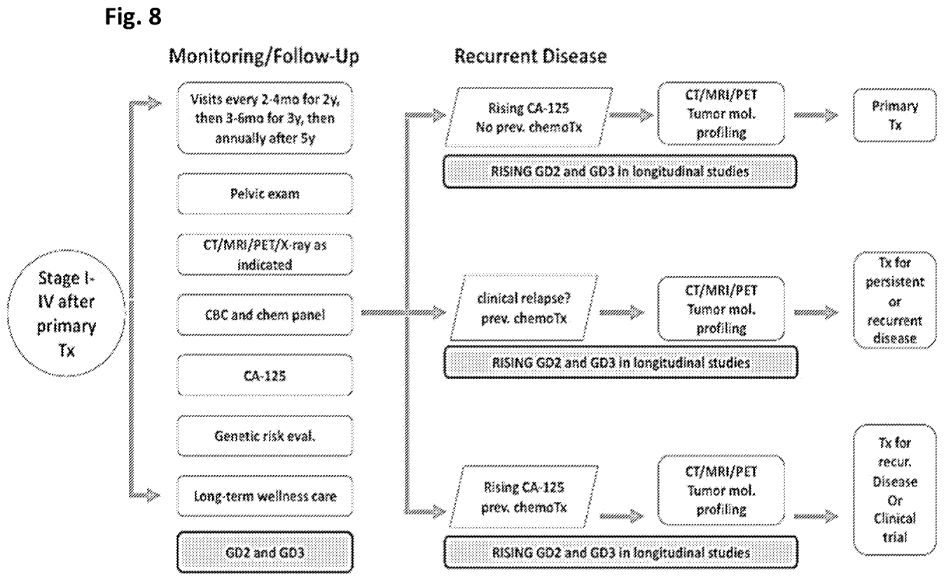

FIG. 8 shows a schematic diagram adapted from the National Comprehensive Cancer Networks Clinical Practice Guidelines in Ovarian Cancer, v3.2019. The current guidelines for Monitoring and Risk assessment are inadequate for early detection or monitoring. Adding the GD2/GD3 tests of the present disclosure (yellow boxes) would detect early and late stage ovarian cancers, many of which are missed by CA-125 standard of care liquid biopsy.

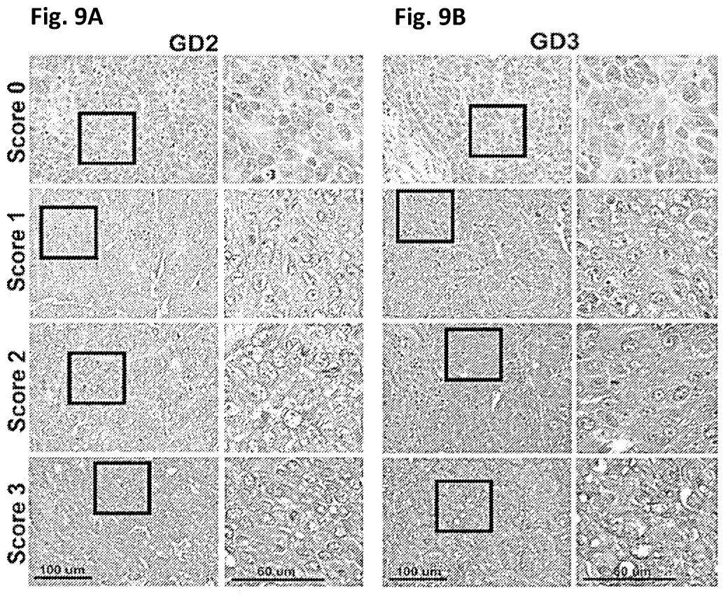

FIG. 9A-FIG. 9B show immunohistochemical detection of tumoral marker gangliosides (TMGs) in ovarian cancer biopsies. Images show representative pictures of anti-GD2 (FIG. 9A) and anti-GD3 (FIG. 9B) antibody staining, scored as “0” (no staining), “1” (weak staining), “2” (moderate staining) and “3” (strong staining). Scores “0” and “1” were considered negative and scores “2” and “3” were deemed positive. The right panels for each column are higher magnification of the areas within the black rectangles.

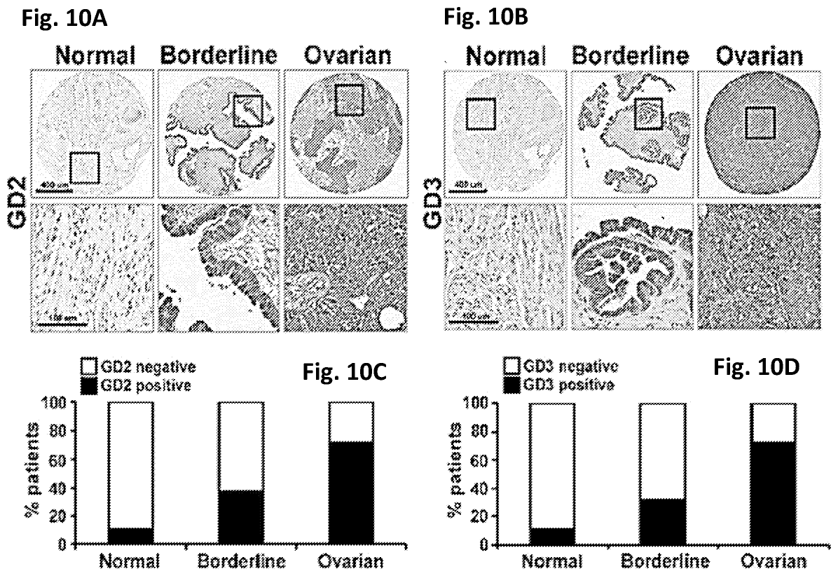

FIG. 10A-FIG. 10D show that tumoral marker gangliosides (TMGs) are highly expressed in ovarian cancer patients. Representative images showing GD2 (FIG. 10A) and GD3 (FIG. 10B) immunohistochemistry in normal, borderline and ovarian tissue biopsies. The bottom panels show a higher magnification of the top panel areas within the black rectangles. GD2 and GD3 specifically stained tumor cells, but not surrounding normal tissue. Staining was mainly localized in the cytoplasmic membrane and cytoplasm. Percentage of normal, borderline and ovarian patients negative (Scores “0” and “1”) or positive (Scores “2” and “3”) for GD2 (FIG. 10C) and GD3 (FIG. 10D). The expression of TMGs is significantly higher in borderline and ovarian patients compared to normal (n=9 normal; n=16 borderline; n=151 ovarian), p<0.0001 (Chi-square).

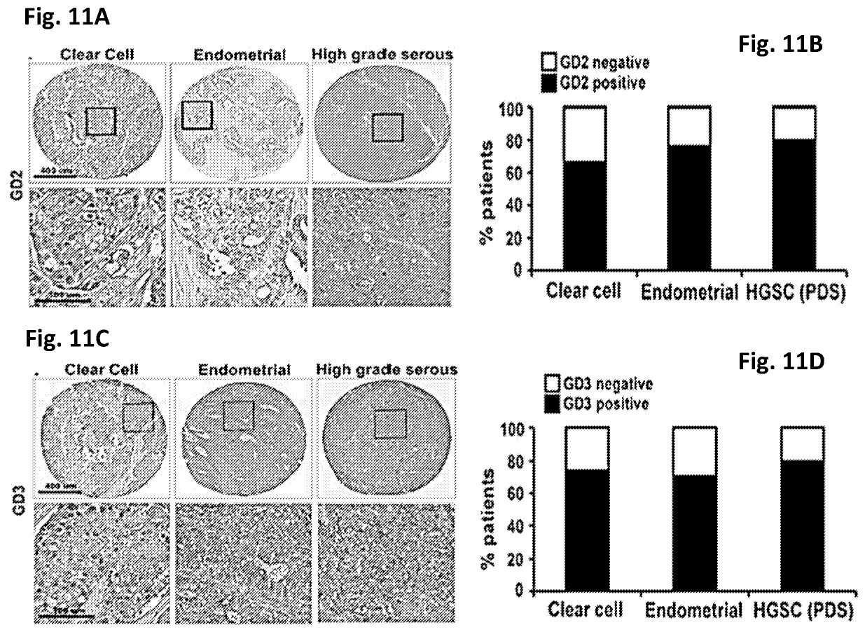

FIG. 11A-FIG. 11D show that tumoral marker gangliosides (TMGs) are highly expressed in different ovarian cancer subtypes. Representative images showing GD2 (FIG. 11A) and GD3 (FIG. 11B) immunohistochemistry in clear cell and endometrial cancer tissue biopsies; and in post-debulking surgery (PDS) cancer tissue biopsies from high grade serous cancer (HGSC) patients. The bottom panels show a higher magnification of the top panel areas within the black rectangles. Percentage of ovarian cancer subtype patients negative (Scores “0” and “1) or positive (Scores “2” and “3”) for GD2 (FIG. 11C) and GD3 (FIG. 11D). The levels of GD2 and GD3 expression are similar among the different ovarian cancer subtypes, although there are minor statistical differences for GD2 staining (p=0.035, Chi-square). No statistical differences were observed for GD3 (n=27 clear cell; n=17 endometrial; n=55 HGSC (PDS)).

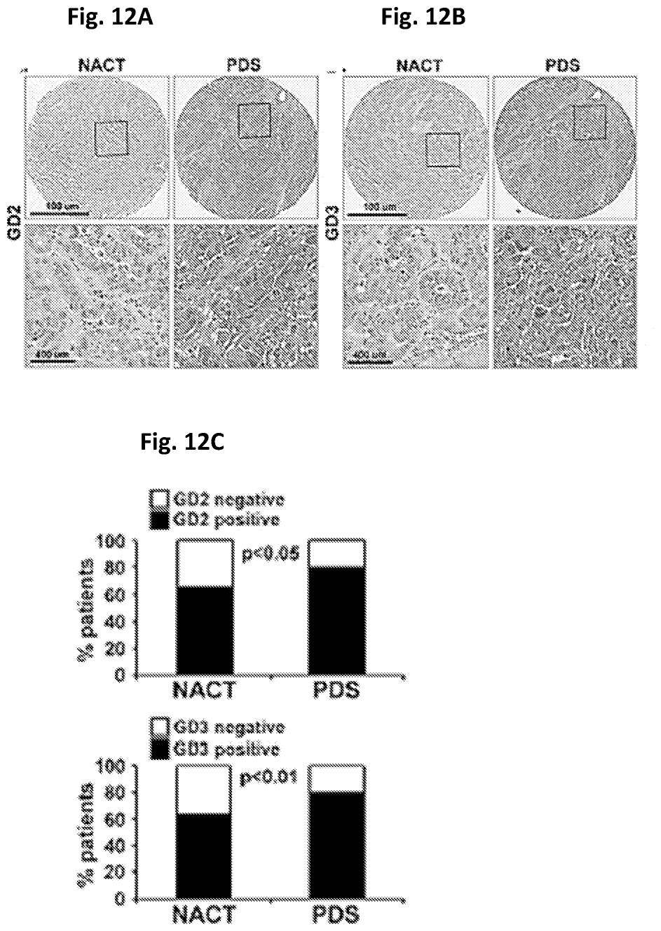

FIG. 12A-FIG. 12C show that tumoral marker gangliosides (TMGs) expression is decreased in HGSC patients treated with neoadjuvant chemotherapy (NACT). Representative images showing GD2 (FIG. 12A) and GD3 (FIG. 12B) immunohistochemistry in HGSC (PDS) or HGSC (NACT). Quantification (in percentage) of positive and negative patients showed that GD2 and GD3 expression is significantly reduced in patients that underwent NACT vs. patients that underwent PDS (n=52 NACT, n=55 PDS). p=0.017 for GD2 and p=0.007 for GD3, (Chi-square).

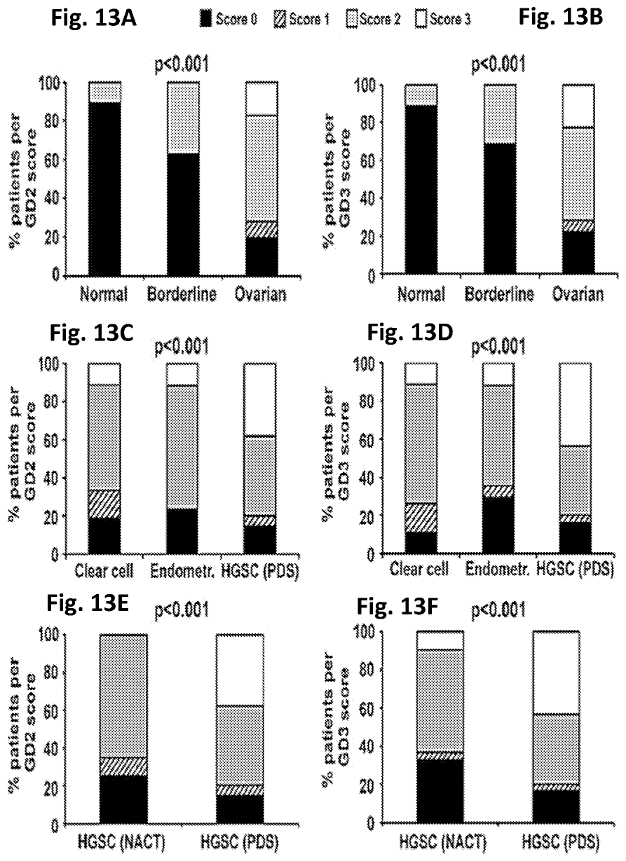

FIG. 13A-FIG. 13F show that higher scores in tumoral marker gangliosides (TMGs) staining (IHC) is associated with more aggressive ovarian cancer subtypes. Quantification (in percentage) of GD2 and GD3 scores in normal patients vs. borderline and ovarian cancer patients (FIG. 13A, FIG. 13B); in ovarian cancer subtypes (FIG. 13C, FIG. 13D) and in HGSC (PDS vs. NACT) (FIG. 13E, FIG. 13F). Ovarian cancer patients showed significantly higher GD2 and GD3 staining scores compared to borderline and normal (FIG. 13A, FIG. 13B). GD2 and GD3 scores were significantly higher in HGSC (PDS) patients vs. clear cell or endometrial cancer subtypes (FIG. 13C, FIG. 13D) or vs. HGCS (NACT) patients (FIG. 13E, FIG. 13F). (n=9 normal; n=16 borderline; n=151 ovarian; from n=151 ovarian, n=27 clear cell; n=17 endometrial; n=55 HGSC (PDS) and n=52 HGSC (NACT). p<0.001 (Chi-square).

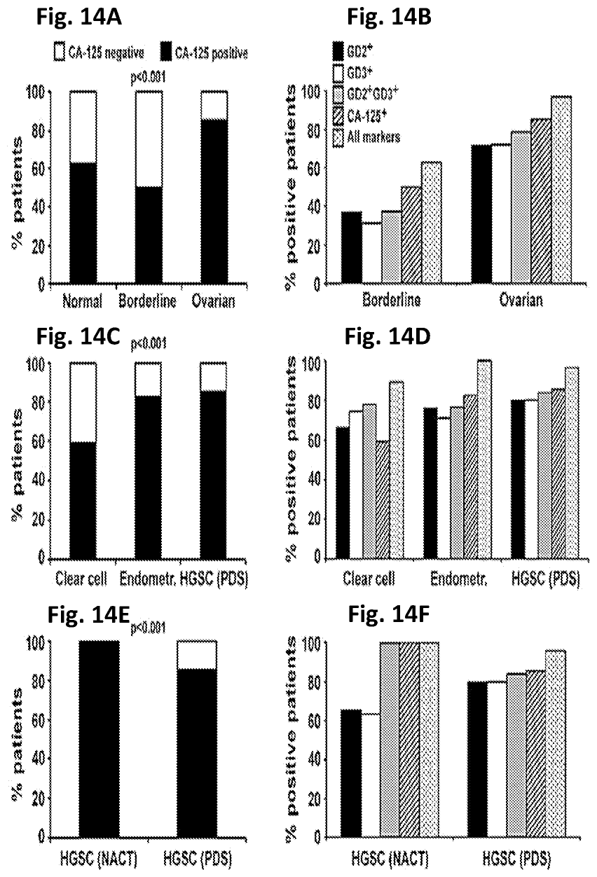

FIG. 14A-FIG. 14F show that tumoral marker gangliosides (TMGs) detected similar percentage of positive cancer patients compared to the CA-125 ovarian cancer marker used in clinic. TMGs were detected using IHC, and CA-125 was detected in liquid biopsies. CA-125 levels are shown in normal patients vs. borderline and ovarian cancer patients (FIG. 14A); in ovarian cancer subtypes (FIG. 14C) and in HGSC (PDS vs. NACT) (FIG. 14E). Although CA-125 levels were statistically different in FIG. 14A, FIG. 14C and FIG. 14E (p<0.001, Chi-square), there was not a direct correlation between aggressiveness of the disease and CA-125 values detected. Quantification of positive patients (in percentage) GD2+, GD3+, GD2+GD3+, CA-125+ or positive for all markers together in borderline vs. ovarian cancer patients (FIG. 14B); in ovarian cancer subtypes (FIG. 14D) and in HGSC (PDS vs. NACT) (FIG. 14F). All markers, individually or combined, achieved similar levels of detection of positive ovarian cancer patients. Females were classified as postmenopausal based on average age (62±6 yrs. for normal; 57±15 yrs. for borderline; 61±12 yrs. for all ovarian; 5530 12 yrs. for clear cell; 60±10 yrs. for endometrial; 63±12 yrs. for HGSC (PDS) and 63±10 yrs. HGSC (NACT). No significant age differences were found among the groups, except for the clear cell group vs. HGSC (PDS) patients (p<0.01, One-Way ANOVA with Bonferroni correction for multiple comparisons) (n=9 normal; n=16 borderline; n=151 ovarian; from n=151 ovarian, n=27 clear cell; n=17 endometrial; n=55 HGSC (PDS) and n=52 HGSC (NACT).

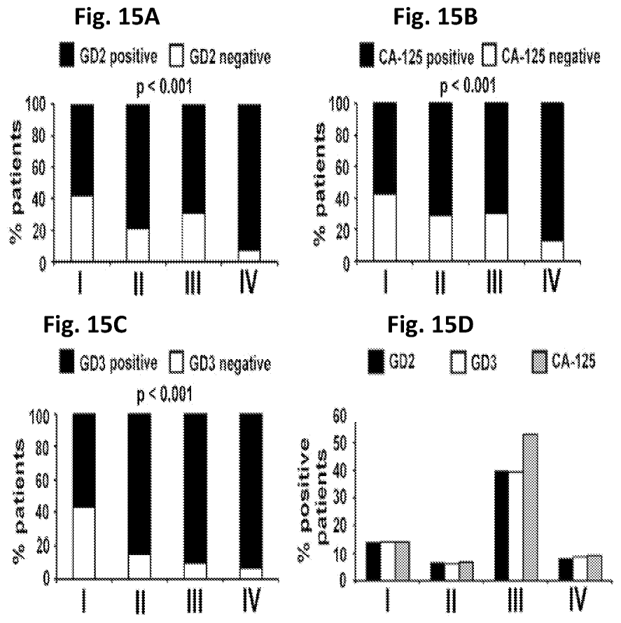

FIG. 15A-FIG. 15D show that tumoral marker gangliosides (TMGs) are more highly expressed in advanced stages of ovarian cancer. Quantification (in percentage) of positive and negative GD2 (FIG. 15A), CA-125 (FIG. 15B) or GD3 (FIG. 15C) at different stages of the disease. TMGs were detected using IHC, and CA-125 was detected in liquid biopsies. Total number of patients (100%) corresponds to the sum of total patients detected in each individual stage (I or II or III or IV). All markers were significantly highly expressed in more advanced stages of ovarian cancer (p<0.001, Chi-square). (FIG. 15D) Comparison of the percentage of positive patients detected by each marker. No significant differences were observed (n=162 patients).

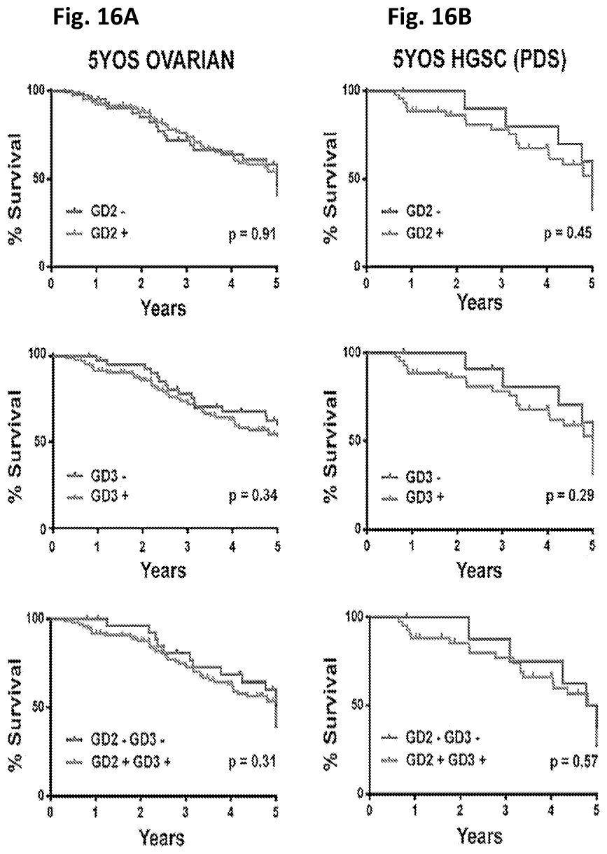

FIG. 16A-FIG. 16B show 5-years overall survival (5YOS). Correlation between tumoral marker gangliosides (TMGs) and 5YOS in (FIG. 16A) ovarian patients and (FIG. 16B) HGSC (PDS). TMGs were detected using IHC. Data are shown as percentage of survival in either GD2+ and GD2− or GD3+ and GD3− or GD2− GD3− and GD2+GD3+. No statistical differences were reported, although, in general, GD2+, GD3+, and GD2+GD3+ patients tend to have a slightly reduced survival compared to GD2−, GD3−, and GD2− GD3− patients (n=151 ovarian in FIG. 16A; n=55 HGSC (PDS) in FIG. 16B).

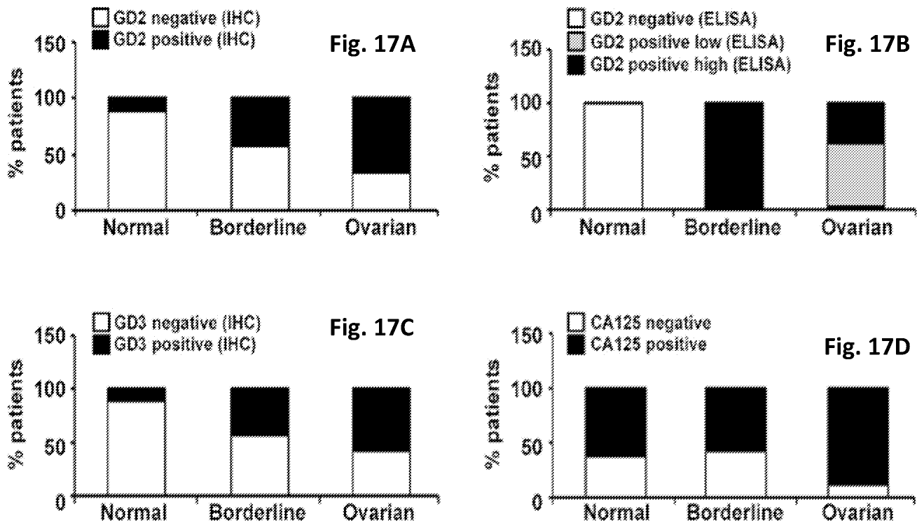

FIG. 17A-FIG. 17D show that detection of GD2 in liquid biopsies by ELISA is a more sensitive ovarian marker than CA-125. TMGs were detected using IHC (FIG. 17A-FIG. 17B), the level of which was matched to the level of TMGs measured in liquid biopsies using ELISA (FIG. 17C). CA-125 (FIG. 17D) was also detected in liquid biopsies using ELISA. Quantification (in percentage) of negative or positive GD2 (FIG. 17A) or GD3 (FIG. 17B) in tissue biopsies from normal, borderline and ovarian cancer patients. The expression of TMGs is significantly higher in borderline and ovarian patients compared to normal (n=9 normal; n=7 borderline; n=33 ovarian), p<0.0001 (Chi-square). (FIG. 17C) GD2 levels analyzed in liquid biopsies from same borderline and ovarian patients represented in (FIG. 17A). GD2 was significantly elevated in ovarian and borderline cancer patients compared to normal patients (n=19 normal, n=7 borderline, n=33 ovarian) (p<0.0001, Chi-square) (FIG. 17D) CA-125 levels in same patients analyzed in (FIG. 17A) and (FIG. 17C). CA-125 levels were significantly higher in ovarian patients compared to normal (n=8 normal, n=7 borderline, n=33 ovarian) (p<0.0001, Chi-square), but not versus borderline. About 40% of borderline patients and 8% of ovarian cancer patients were negative for CA-125, but GD2 positive by ELISA indicating that GD2 is a significant superior marker, mainly for early stages of the disease.

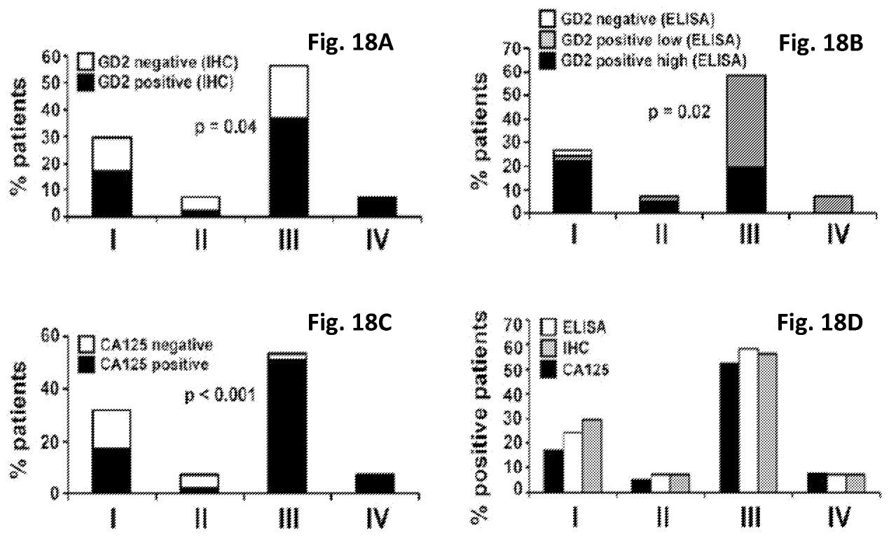

FIG. 18A-FIG. 18D show comparative detection of ovarian markers in different cancer stages. Percentage of positive or negative patients for GD2 detected by IHC (FIG. 18A) by ELISA (FIG. 18B) or for CA-125 (FIG. 18C). The percentage of GD2 and CA-125 positive patients was significantly higher versus negative patients (p=0.04 for FIG. 18A, p=0.02 for FIG. 18B and p<0.0001 for C, Chi-square). (FIG. 18D) Comparison of the percentage of positive patients detected by each technique. Total number of patients (100%) corresponds to the sum of patients detected in all stages (I+II+III+IV). No significant differences were observed (n=41 patients).

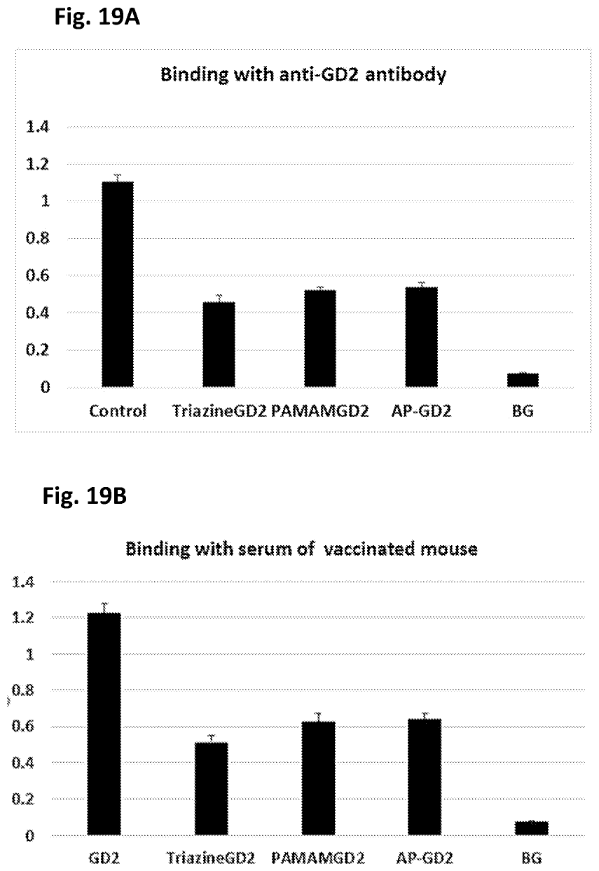

FIG. 19A-19D show the results of direct ELISA experiments, which demonstrate that both Triazine-triGD2 and Triazine-triGD3 were recognized by anti-GD2 or anti-GD3 mAbs, respectively. FIG. 19A shows binding of an anti-GD2 antibody. FIG. 19B shows binding of serum from a vaccinated mouse to GD2 antigens. FIG. 19C shows binding with an anti-GD3 antibody. FIG. 19D shows biding of serum from a vaccinated mouse to GD3 antigens.

FIGS. 20A and 20B show the results of competitive ELISA experiments that demonstrated both Triazine-triGD2 and Triazine-triGD3 were recognized by anti-GD2 or anti-GD3 mAbs, respectively; resulting in competition of the mAbs binding to GD2 or GD3 immobilized onto the plates. FIG. 20A shows binding of monoclonal antibodies to triazine-GD2. FIG. 20B shows binding of monoclonal antibodies to triazine-GD3.

### DETAILED DESCRIPTION OF THE INVENTION

Gangliosides are a family of >40 different sialic acid-containing glycosphingolipids. Each glycan tree is structurally unique and defines each ganglioside by name. Some gangliosides such as GM1 are normal and ubiquitous. Other gangliosides such as GD2 and GD3 are tumor-markers (Table 3). They are low/absent in normal cells, and are expressed at high levels in cancer. Hence, GD2 and GD3 are etiological biomarkers (i.e., those with indispensable function for the cancer), which are preferred because cancers do not easily downregulate expression of the marker.

GD2 and GD3 regulate membrane fluidity, raft size, and function, and provide tumors with advantages in growth/metastasis, immune evasion, and blockade. The lipid tails are embedded in the outer leaflet of cell membranes and are variable in length. The biological relevance of the variable lipid length is unknown.

GD2 or GD3 provide for stable, invariant, and non-mutating targets, expression is conserved across mammalian species (the glycan tree is identical), expression is homogeneous and uniform in cell lines and in primary tumors, and density of expression does not downregulate in the surviving tumor cells after chemotherapy. In addition to being present on the tumor cell surface, GD2 and GD3 can be shed into the extracellular environment.

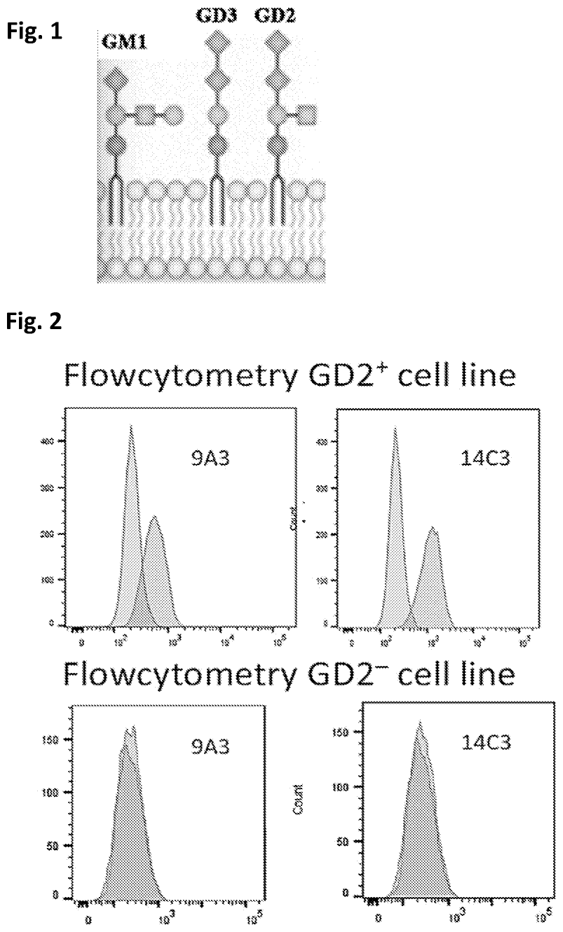

However, studies of expression of GD2 and GD3 in tissues or in circulation only reported few patient samples. Assays to detect GD2 or GD3 that may be expressed in tissue or in serum are not quantitative or standardized. Studies used Thin Layer Chromatography (TLC) or lipid-associated sialic acids (LASAs) that only yield estimations, and resulted in contradictory conclusions. There is a scarcity of tools to study GD2 or GD3. Even after 40 years of research there are very few mAbs against GD2 or GD3 with which to design assays and many are not specific, most are suboptimal and are cross-reactive given the small differences in ganglioside structures (FIG. 1). Since GD2 and GD3 are glycolipids and the products can be generated via multiple biosynthetic pathways and enzymes, monitoring mutations or mRNA expression is not feasible. GD2 and GD3 remain underexploited for diagnosis of any cancer.

Accordingly, the present disclosure provides monoclonal antibodies, and antigen-binding fragments thereof, that specifically bind to a ganglioside (e.g., GD2 or GD3), as well as immunoglobulins, polypeptides, nucleic acids thereof, and methods of using such antibodies for diagnostic and prognostic purposes. Further provided herein are novel mass spectrometry-based methods to detect the presence, level, and/or lipid length of a ganglioside (e.g., GD2, GD3, GM2, and/or GD1). The discovery presented herein that the heterogeneity of the lipid length of gangliosides changes in cancer patients provides a novel biomarker and utility of such biomarker for cancer diagnosis and prognosis.

### Definitions

The articles “a” and “an” are used herein to refer to one or to more than one (i.e. to at least one) of the grammatical object of the article. By way of example, “an element” means one element or more than one element.

As used herein, the term “composite antibody” refers to an antibody which has variable regions comprising germline or non-germline immunoglobulin sequences from two or more unrelated variable regions. Additionally, the term “composite, human antibody” refers to an antibody which has constant regions derived from human germline or non-germline immunoglobulin sequences and variable regions comprising human germline or non-germline sequences from two or more unrelated human variable regions.

A molecule is “fixed” or “affixed” to a substrate if it is covalently or non-covalently associated with the substrate such the substrate can be rinsed with a fluid (e.g. standard saline citrate, pH 7.4) without a substantial fraction of the molecule dissociating from the substrate.

The terms “CDR”, and its plural “CDRs,” refer to a complementarity determining region (CDR) of which three make up the binding character of a light chain variable region (CDR-L1, CDR-L2 and CDR-L3) and three make up the binding character of a heavy chain variable region (CDR-H1, CDR-H2 and CDR-H3). CDRs contribute to the functional activity of an antibody molecule and are separated by amino acid sequences that comprise scaffolding or framework regions. The exact definitional CDR boundaries and lengths are subject to different classification and numbering systems. CDRs may therefore be referred to by Kabat, Chothia, contact or any other boundary definitions. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat, Chothia, and/or MacCallum et al., (Kabat et al., in “Sequences of Proteins of Immunological Interest,” 5th Edition, U.S. Department of Health and Human Services, 1992; Chothia et al. (1987) J. Mol. Biol. 196, 901; and MacCallum et al., J. Mol. Biol. (1996) 262, 732, each of which is incorporated by reference in its entirety).

As used herein, the term “Fc region” is used to describe a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. Suitable native-sequence Fc regions for use in the antibodies of the present invention include human IgG1, IgG2 (IgG2A, IgG2B), IgG3 and IgG4.

As used herein, the term “KD” is intended to refer to the dissociation equilibrium constant of a particular antibody-antigen interaction. The binding affinity of antibodies of the disclosed invention may be measured or determined by standard antibody-antigen assays, for example, competitive assays, saturation assays, or standard immunoassays such as ELISA or RIA.

The lipid length refers the length of the lipid tails of a ganglioside that are important for diagnostics. The lipid tails are embedded in the outer leaflet of cell membranes. The lipids can be variable in length. Notably, these differences occur when comparing amongst gangliosides as well as within a single ganglioside. Thus, the lipid heterogeneity and changes thereof are important for diagnostics and methods described herein.

The term “minimal residual disease” is art recognized, and is used to describe a small number of cancer cells in the body during or after cancer treatment, when the patient is in remission. The number of remaining cells may be so small that they do not cause any physical signs or symptoms and often cannot even be detected through traditional methods. It is a major cause of relapse of cancer.

The term “neoadjuvant therapy” refers to a treatment given before the primary treatment. Examples of neoadjuvant therapy can include chemotherapy, radiation therapy, and hormone therapy.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

The term “preventing” is art-recognized, and when used in relation to a condition, such as a viral/bacterial infection or a disease such as cancer is well understood in the art, and includes administration of a treatment, e.g., a composition which reduces the frequency of, or delays the onset of, symptoms of a medical condition in a subject relative to a subject which does not receive the treatment. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

The term “remission” is art recognized, and refers to a condition in which the signs and symptoms of the cancer are reduced.

As used herein, “subject” refers to any healthy animal, mammal or human, or any animal, mammal or human afflicted with a cancer. The term “subject” is interchangeable with “patient”. The term “non-human animal” includes all vertebrates, e.g., mammals and non-mammals, such as non-human primates, sheep, dog, cow, chickens, amphibians, reptiles, etc.

A “therapeutically effective amount” of a compound is an amount capable of producing a medically desirable result in a treated patient, e.g., induce immune response against a ganglioside, decrease tumor burden, decrease the growth of tumor cells, or alleviate any symptom associated with cancer, with an acceptable benefit: risk ratio, preferably in a human or non-human mammal.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal), then the treatment is prophylactic (i.e., it protects the host against developing the unwanted condition); whereas, if it is administered after manifestation of the unwanted condition, the treatment is therapeutic (i.e., it is intended to diminish, ameliorate, or stabilize the existing unwanted condition or side effects thereof).

### Antigen-Binding Proteins

Provided herein are antigen-binding proteins that bind to a ganglioside (e.g., GD2 or GD3). In various embodiments, the antigen binding proteins bind to the carbohydrate portion of the ganglioside (e.g., GD2 or GD3). The antigen-binding proteins of the present disclosure can take any one of many forms of antigen-binding proteins known in the art. In various embodiments, the antigen-binding proteins of the present disclosure take the form of an antibody, or antigen-binding antibody fragment, or an antibody protein product.

In various embodiments of the present disclosure, the antigen-binding protein comprises, consists essentially of, or consists of an antibody. As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. The constant region allows the antibody to recruit cells and molecules of the immune system. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4th ed. Elsevier Science Ltd./Garland Publishing, (1999)).

Unless otherwise specified here within, antibody or antibodies broadly encompass naturally-occurring forms of antibodies (e.g. IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody. An antibody refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH₁, CH₂ and CH₃. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. Framework or FR residues are those variable-domain residues other than the hypervariable residues as herein indicated. The variable regions of the heavy and light chains contain a binding domain that interacts with an antigen.

The general structure and properties of CDRs of antibodies have been described in the art. Briefly, in an antibody scaffold, the CDRs are embedded within a framework in the heavy and light chain variable region where they constitute the regions largely responsible for antigen binding and recognition. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342:877-883), within a framework region (designated framework regions 1-4, FR1, FR2, FR3, and FR4, by Kabat et al., 1991; see also Chothia and Lesk, 1987, supra). In a related embodiment, the residues of the framework are altered. The heavy chain framework regions which can be altered lie within regions designated H-FR1, H-FR2, H-FR3 and H-FR4, which surround the heavy chain CDR residues, and the residues of the light chain framework regions which can be altered lie within the regions designated L-FR1, L-FR2, L-FR3 and L-FR4, which surround the light chain CDR residues. An amino acid within the framework region may be replaced, for example, with any suitable amino acid identified in a human framework or human consensus framework.

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in various embodiments, the antibody is an antibody of isotype IgA, IgD, IgE, IgG, or IgM, including any one of IgG1, IgG2, IgG3 or IgG4. In various aspects, the antibody comprises a constant region comprising one or more amino acid modifications, relative to the naturally-occurring counterpart, in order to improve half-life/stability or to render the antibody more suitable for expression/manufacturability. In various instances, the antibody comprises a constant region wherein the C-terminal Lys residue that is present in the naturally-occurring counterpart is removed or clipped.

The antibody can be a monoclonal antibody. In some embodiments, the antibody comprises a sequence that is substantially similar to a naturally-occurring antibody produced by a mammal, e.g., mouse, rabbit, goat, horse, chicken, hamster, human, and the like. In this regard, the antibody can be considered as a mammalian antibody, e.g., a mouse antibody, rabbit antibody, goat antibody, horse antibody, chicken antibody, hamster antibody, human antibody, and the like. In certain aspects, the antigen-binding protein is an antibody, such as a human antibody. In certain aspects, the antigen-binding protein is a chimeric antibody or a humanized antibody. The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence. Information, including sequence information for human antibody heavy and light chain constant regions is publicly available through the Uniprot database as well as other databases well-known to those in the field of antibody engineering and production. For example, the IgG2 constant region is available from the Uniprot database as Uniprot number P01859, incorporated herein by reference.

Antibody as used herein also includes antigen-binding portion of an antibody. The antigen-binding portion refers to one or more fragments of an antibody that retain the ability to specifically bind to an antigen (e.g., a ganglioside). It has been shown that the antigen-binding function of an antibody can be performed by fragments of a full-length antibody. Examples of binding fragments encompassed within the antigen-binding portion of an antibody include (i) a Fab fragment, a monovalent fragment consisting of the VL, VH, CL and CHI domains; (ii) a F(ab′) 2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16:778). Such single chain antibodies are also intended to be encompassed within the antigen-binding portion of an antibody.

An antibody can be cleaved into fragments by enzymes, such as, e.g., papain and pepsin. Papain cleaves an antibody to produce two Fab fragments and a single Fc fragment. Pepsin cleaves an antibody to produce a F (ab′) 2 fragment and a pFc′ fragment. In various aspects of the present disclosure, the antigen-binding protein of the present disclosure is an antigen-binding fragment of an antibody (a.k.a., antigen-binding antibody fragment, antigen-binding fragment, antigen-binding portion). In various instances, the antigen-binding antibody fragment is a Fab fragment or a F (ab′) 2 fragment.

The architecture of antibodies has been exploited to create a growing range of alternative antibody formats that spans a molecular-weight range of at least or about 12-150 kDa and has a valency (n) range from monomeric (n=1), to dimeric (n=2), to trimeric (n=3), to tetrameric (n=4), and potentially higher; such alternative antibody formats are referred to herein as “antibody protein products”. Antibody protein products include those based on the full antibody structure and those that mimic antibody fragments which retain full antigen-binding capacity, e.g., scFvs, Fabs and VHH/VH (discussed below). The smallest antigen-binding fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment [fragment, antigen-binding]. Both scFv and Fab fragments can be easily produced in host cells, e.g., prokaryotic host cells. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4 (5): 586-591 (2012).

Other antibody protein products include a single chain antibody (SCA); a diabody; a triabody; a tetrabody; bispecific or trispecific antibodies, and the like. Bispecific antibodies can be divided into five major classes: BsIgG, appended IgG, bispecific antibody (BsAb) fragments, bispecific fusion proteins, and BsAb conjugates. See, e.g., Spiess et al., Molecular Immunology 67 (2) Part A: 97-106 (2015).

In various aspects, the antigen-binding protein of the present disclosure comprises, consists essentially of, or consists of any one of these antibody protein products. In various aspects, the antigen-binding protein of the present disclosure comprises, consists essentially of, or consists of any one of a Fab VHH/VH, Fv fragment, ds-scFv, scFab, Fv, F(ab′) 2, Fab′, dsFv, scFv, sc (Fv) 2, dimeric antibody, multimeric antibody (e.g., a diabody, triabody, tetrabody), miniAb, peptibody VHH/VH of camelid heavy chain antibody, sdAb, diabody; a triabody; and a tetrabody.

In various instances, the antigen-binding protein of the present disclosure is an antibody protein product in monomeric form, or polymeric, oligomeric, or multimeric form.

In certain aspects, provided herein is a monoclonal antibody, or antigen-binding fragment thereof, wherein the monoclonal antibody specifically binds to the carbohydrate portion of a ganglioside. In some embodiments, the ganglioside is (a) GD2, (b) GD3, or (c) GD2 and GD3.

In various embodiments, an anti-ganglioside antibody (against GD2 or GD3) or antibody variant thereof is selected from the group consisting of a human antibody, a humanized antibody, a chimeric antibody, a monoclonal antibody, a recombinant antibody, an antigen-binding antibody fragment, a single chain antibody, a monomeric antibody, a diabody, a triabody, a tetrabody, Fv, F(ab′) 2, Fab′, dsFv, scFv, sc (Fv) 2, an IgG1 antibody, an IgG2 antibody, an IgG3 antibody, and an IgG4 antibody.

In certain aspects, provided herein is a monoclonal antibody, or antigen-binding fragment thereof, wherein the monoclonal antibody comprises a) a heavy chain complementarity determining region (CDR) sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to a heavy chain CDR sequence selected from the group consisting of the sequences listed in Table 1; and/or b) a light chain CDR sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to a light chain CDR sequence selected from the group consisting of the sequences listed in Table 1.

In certain aspects, a monoclonal antibody, or antigen-binding fragment thereof, wherein the monoclonal antibody comprises a) a heavy chain variable domain (VH) with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to a VH sequence selected from the group consisting of the VH sequences listed in Table 2; and/or b) a light chain variable domain (VL) sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to a VL sequence selected from the group consisting of the VL sequences listed in Table 2, is provided.

In certain aspects, a monoclonal antibody, or antigen-binding fragment thereof, wherein the monoclonal antibody, or antigen-binding fragment thereof, comprises a) a combination of a heavy chain CDR1, CDR2, and CDR3 as set forth in Table 1, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; and/or b) a combination of a light chain CDR1, CDR2, and CDR3 as set forth in Table 1, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity, is provided.

In certain aspects, provided herein is a monoclonal antibody, or antigen-binding fragment thereof, wherein the monoclonal antibody, or antigen-binding fragment thereof, comprises: a) a VH sequence as set forth in Table 2, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; and/or b) a VL sequence as set forth in Table 2, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity.

In some embodiments, a monoclonal antibody, or antigen-binding fragment thereof, wherein the monoclonal antibody comprises a) a VH sequence selected from the group consisting of the VH sequences listed in Table 2; and/or b) a VL sequence selected from the group consisting of the VL sequences listed in Table 2, is provided.

In some embodiments, the monoclonal antibody or antigen-binding fragment thereof comprises six CDR amino acid sequences selected from: a) SEQ ID NOs: 2, 4, 6, 8, 10, and 12 (clone 4), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; b) SEQ ID NOs: 14, 16, 18, 20, 22, and 24 (clone 6), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; c) SEQ ID NOs: 26,28, 30, 32, 34, and 36 (clone 7), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; d) SEQ ID NOs: 38, 40, 42, 44, 46, and 48 (clone 8), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; e) SEQ ID NOs: 50, 52, 54, 56, 58, and 60 (clone 9), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; f) SEQ ID NOs: 62, 64, 66, 68, 70, and 72 (clone 10), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; g) SEQ ID NOs: 74, 76, 78, 80, 82, and 84 (clone 13), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; h) SEQ ID NOs: 86, 88, 90, 92, 94, and 96 (clone 14), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; i) SEQ ID NOs: 98, 100, 102, 104, 106, and 108 (clone 15), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; j) SEQ ID NOs: 110, 112, 114, 116, 118, and 120 (clone 17), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; k) SEQ ID NOs: 122, 124, 126, 128, 130, and 132 (clone 18), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; and 1) SEQ ID NOs: 134, 136, 138, 140, 142, and 144 (clone 19), or a variant sequence thereof which differs by only one or two amino acids, or which has at least or about 85% sequence identity.

In some embodiments, the monoclonal antibody or antigen-binding fragment thereof comprises the VH and VL amino acid sequences selected from: a) SEQ ID NOs: 146 and 148, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; b) SEQ ID NOs: 150 and 152, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; c) SEQ ID NOs: 154 and 156, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; d) SEQ ID NOs: 158 and 160, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; e) SEQ ID NOs: 162 and 164, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; f) SEQ ID NOs: 166 and 168, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; g) SEQ ID NOs: 170 and 172, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; h) SEQ ID NOs: 174 and 176, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; i) SEQ ID NOs: 178 and 180, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; j) SEQ ID NOs: 182 and 184, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; k) SEQ ID NOs: 186 and 188, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; and 1) SEQ ID NOs: 190 and 192, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity.

Numerous embodiments are further provided that can be applied to any aspect of the present invention described herein. For example, in some embodiments, the monoclonal antibody, or antigen-binding fragment thereof, is chimeric, humanized, composite, murine, or human. In some embodiments, the monoclonal antibody, or antigen-binding fragment thereof, comprises an immunoglobulin heavy chain constant domain selected from the group consisting of IgG, IgG1, IgG2, IgG2A, IgG2B, IgG3, IgG4, IgA, IgM, IgD, and IgE constant domains. In some embodiments, the monoclonal antibody, or antigen-binding fragment thereof, is detectably labeled, comprises an effector domain, comprises an Fc domain, and/or is selected from the group consisting of Fv, F(ab′) 2, Fab′, dsFv, scFv, sc (Fv) 2 fragments, diabodies, bivalent, multivalent, and bifunctional engineered constructs. In some embodiments, the monoclonal antibody, or antigen-binding fragment thereof, is obtainable from hybridoma. In some embodiments, the monoclonal antibody, or antigen-binding fragment thereof, specifically binds a ganglioside (e.g., GD2 or GD3). In some embodiments, the monoclonal antibody, or antigen-binding fragment thereof, specifically binds a carbohydrate portion of a ganglioside (e.g., GD2 or GD3).

In certain aspects, a conjugate comprising the monoclonal antibody, or antigen-binding fragment thereof, described herein is provided. In some embodiments, the conjugate comprises a detectable moiety (e.g., a fluorophore, an enzyme, a radioisotope, etc.).

In certain aspects, an immunoglobulin heavy and/or light chain selected from the group consisting of immunoglobulin heavy and light chain sequences listed in Table 2, is provided.

### Sequence Identity/Homology

Function-conservative variants are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A function-conservative variant also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared.

The percent identity between two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available on the world wide web at the GCG company website), using a NWSgapdna. CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11 17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48): 444 453 (1970)) algorithm which has been incorporated into the GAP program in the GCG software package (available on the world wide web at the GCG company website), using either a Blosum 62 matrix or a PAM250 matrix, and a gap weight of 16, 14, 12, 10, 8, 6, or 4 and a length weight of 1, 2, 3, 4, 5, or 6.

The nucleic acid and protein sequences of the present invention can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403 10. BLAST nucleotide searches can be performed with the NBLAST program, score=100, wordlength=12 to obtain nucleotide sequences homologous to the nucleic acid molecules of the present invention. BLAST protein searches can be performed with the XBLAST program, score=50, wordlength=3 to obtain amino acid sequences homologous to the protein molecules of the present invention. To obtain gapped alignments for comparison purposes, Gapped BLAST can be utilized as described in Altschul et al., (1997) Nucleic Acids Res. 25 (17): 3389 3402. When utilizing BLAST and Gapped BLAST programs, the default parameters of the respective programs (e.g., XBLAST and NBLAST) can be used (available on the world wide web at the NCBI website).

### Sequences

As used herein, coding region refers to regions of a nucleotide sequence comprising codons which are translated into amino acid residues, whereas noncoding region refers to regions of a nucleotide sequence that are not translated into amino acids (e.g., 5′ and 3′ untranslated regions).

Complement [to] or complementary refers to the broad concept of sequence complementarity between regions of two nucleic acid strands or between two regions of the same nucleic acid strand. It is known that an adenine residue of a first nucleic acid region is capable of forming specific hydrogen bonds (base pairing) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least or about 50%, and preferably at least or about 75%, at least or about 90%, or at least or about 95% of the nucleotide residues of the first portion are capable of base pairing with nucleotide residues in the second portion. In other embodiments, all nucleotide residues of the first portion are capable of base pairing with nucleotide residues in the second portion.

A nucleic acid is operably linked when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (-0.9); tyrosine (−1.3); proline (−1.6); histidine (−3.2); glutamate (−3.5); glutamine (−3.5); aspartate (<RTI 3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well-known to those of skill in the art and include: arginine and lysine; glutamate and aspartate; serine and threonine; glutamine and asparagine; and valine, leucine and isoleucine.

In view of the foregoing, the nucleotide sequence of a DNA or RNA can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

TABLE 1
Exemplary sequence of the CDRs of anti-ganglioside monoclonal antibodies

| Clone | | CDR | | Amino Acid |
|---|---|---|---|---|
| No. | Binds: | No. | cDNA Sequence | Sequence |
| 4 | GD2/GD3 | HC- | GGCTACACATTTACCAGGTACTGG | GYTFTRYW |
| Mouse | | CDR1 | (SEQ ID NO: 1) | (SEQ ID NO: 2) |
| mAb | | HC- | ATTTATCCTGGAAATAGTGATACT | IYPGNSDT |
| | | CDR2 | (SEQ ID NO: 3) | (SEQ ID NO: 4) |
| | | HC- | GCAAGATCCGATGGTCCTATGGACTAC | ARSDGPMDY |
| | | CDR3 | (SEQ ID NO: 5) | (SEQ ID NO: 6) |
| | | LC- | GAAAGTGTTGATAATTATGGCATCAGTT | ESVDNYGISF |
| | | CDR1 | TT | (SEQ ID NO: 8) |
| | | | (SEQ ID NO: 7) | |
| | | LC- | GCTGCATCC | AAS |
| | | CDR2 | (SEQ ID NO: 9) | (SEQ ID NO: 10) |
| | | LC- | CAGCAAAGTAAGGAGGTTCCGTTCACG | QQSKEVPFT |
| | | CDR3 | (SEQ ID NO: 11) | (SEQ ID NO: 12) |
| 6 | GD3 | HC- | ACCTATGGAATGAGC | TYGMS |
| Mouse | | CDR1 | (SEQ ID NO: 13) | (SEQ ID NO: 14) |
| IgG | | HC- | TGGATAAACACATATACTGGAGTGCCAA | WINTYTGVPTYGDDFKG |
| Kappa | | CDR2 | CATATGGTGATGACTTCAAGGGA | (SEQ ID NO: 16) |
| | | | (SEQ ID NO: 15) | |
| | | HC- | TGGTTACGCCACCATGCTATGGACTAC | WLRHHAMDY |
| | | CDR3 | (SEQ ID NO: 17) | (SEQ ID NO: 18) |
| | | LC- | AAGGCCAGTGAGAATGTGGTTACTTATG | KASENVVTYVS |
| | | CDR1 | TTTCC | (SEQ ID NO: 20) |
| | | | (SEQ ID NO: 19) | |
| | | LC- | GGGGCATCCAACCGGTACACT | GASNRYT |
| | | CDR2 | (SEQ ID NO: 21) | (SEQ ID NO: 22) |
| | | LC- | GGACAGGGTTACAGCTATCCGTACACG | GQGYSYPYT |
| | | CDR3 | (SEQ ID NO: 23) | (SEQ ID NO: 24) |
| 7 | GD2/GD3 | HC- | GGATTCACTTTTAGTGACGCCTGG | GFTFSDAW |
| Mouse | | CDR1 | (SEQ ID NO: 25) | (SEQ ID NO: 26) |
| mAb | | HC- | ATTAGAAACAAAGCTAATAATCATGCG | IRNKANNHAT |
| | | CDR2 | ACA | (SEQ ID NO: 28) |
| | | | (SEQ ID NO: 27) | |
| | | HC- | ACCAGGCGACATGATTCCTACTTTGACT | TRRHDSYFDY |
| | | CDR3 | AC | (SEQ ID NO: 30) |
| | | | (SEQ ID NO: 29) | |
| | | LC- | CAGGATGTGGATACTGCT | QDVDTA |
| | | CDR1 | (SEQ ID NO: 31) | (SEQ ID NO: 32) |
| | | LC- | TGGGCATCC | WAS |
| | | CDR2 | (SEQ ID NO: 33) | (SEQ ID NO: 34) |
| | | LC- | CAGCAATATCGCAGCTATCCTCTCACG | QQYRSYPLT |
| | | CDR3 | (SEQ ID NO: 35) | (SEQ ID NO: 36) |
| 8 | GD2/GD3 | HC- | GGCTACACATTTACCAGTTACTGG | GYTFTSYW |
| Mouse | | CDR1 | (SEQ ID NO: 37) | (SEQ ID NO: 38) |
| mAb | | HC- | ATTTATCCTGGAAAAAGTGGTACT | IYPGKSGT |
| | | CDR2 | (SEQ ID NO: 39) | (SEQ ID NO: 40) |
| | | HC- | ACAAGATCCGATGGTCCTATGGACTAC | TRSDGPMDY |
| | | CDR3 | (SEQ ID NO: 41) | (SEQ ID NO: 42) |
| | | LC- | GAGAGTGTTGATAATTATGACATTAGTT | ESVDNYDISF |
| | | CDR1 | TT | (SEQ ID NO: 44) |
| | | | (SEQ ID NO: 43) | |
| | | LC- | GCTGCATCC | AAS |
| | | CDR2 | (SEQ ID NO: 45) | (SEQ ID NO: 46) |
| | | LC- | CAGCAAAGTAAGGAGGTTCCGTACACG | QQSKEVPYT |
| | | CDR3 | (SEQ ID NO: 47) | (SEQ ID NO: 48) |
| 9 | GD2/GD3 | HC- | GACTACAACATGGAC | DYNMD |
| Mouse | | CDR1 | (SEQ ID NO: 49) | (SEQ ID NO: 50) |
| IgG | | HC- | GATATTAATCCTAACAATGGTGGTACTA | DINPNNGGTIYNQKFKG |
| Kappa | | CDR2 | TCTACAACCAGAAGTTCAAGGGC | (SEQ ID NO: 52) |
| | | | (SEQ ID NO: 51) | |
| | | HC- | TCGGGGATCTACTATGATTACGCCTGGT | SGIYYDYAWFPY |
| | | CDR3 | TTCCTTAC | (SEQ ID NO: 54) |
| | | | (SEQ ID NO: 53) | |
| | | LC- | AGTGCAAGTCAGGGCATTAGCAATTATT | SASQGISNYLN |
| | | CDR1 | TAAAC | (SEQ ID NO: 56) |
| | | | (SEQ ID NO: 55) | |
| | | LC- | TACACATCAAGTTTACACTCA | YTSSLHS |
| | | CDR2 | (SEQ ID NO: 57) | (SEQ ID NO: 58) |
| | | LC- | CAGCAGTATAGTAAGCTTCCTCCTACG | QQYSKLPPT |
| | | CDR3 | (SEQ ID NO: 59) | (SEQ ID NO: 60) |
| 10 | GD2/GD3 | HC- | GACTACAACATGGAC | DYNMD |
| Mouse | | CDR1 | (SEQ ID NO: 61) | (SEQ ID NO: 62) |
| IgM | | HC- | GATATTAATCCTAACAATGGTGGTACTA | DINPNNGGTIYNQKFKG |
| Kappa | | CDR2 | TCTACAACCAGAAGTTCAAGGGC | (SEQ ID NO: 64) |
| | | | (SEQ ID NO: 63) | |
| | | HC- | TCGGGGATCTACTATGATTACGCCTGGT | SGIYYDYAWFPY |
| | | CDR3 | TTCCTTAC | (SEQ ID NO: 66) |
| | | | (SEQ ID NO: 65) | |
| | | LC- | AGTGCAAGTCAGGGCATTAGCAATTATT | SASQGISNYLN |
| | | CDR1 | TAAAC | (SEQ ID NO: 68) |
| | | | (SEQ ID NO: 67) | |
| | | LC- | TACACATCAAGTTTACACTCA | YTSSLHS |
| | | CDR2 | (SEQ ID NO: 69) | (SEQ ID NO: 70) |
| | | LC- | CAGCAGTATAGTAAGCTTCCTCCTACG | QQYSKLPPT |
| | | CDR3 | (SEQ ID NO: 71) | (SEQ ID NO: 72) |
| 13 | GD2/GD3 | HC- | GACTATGAAATGCAC | DYEMH |
| Mouse | | CDR1 | (SEQ ID NO: 73) | (SEQ ID NO: 74) |
| IgM | | HC- | GCTATTGATCCTGAAACTGGTGGTACTG | AIDPETGGTAYNQKFKG |
| Kappa | | CDR2 | CCTACAATCAGAAGTTCAAGGGC | (SEQ ID NO: 76) |
| | | | (SEQ ID NO: 75) | |
| | | HC- | AGCTGGGACGGAGACTAC | SWDGDY |
| | | CDR3 | (SEQ ID NO: 77) | (SEQ ID NO: 78) |
| | | LC- | AAGGCCAGTCAGAATGTGGGTACTAAT | KASQNVGTNVA |
| | | CDR1 | GTAGCC | (SEQ ID NO: 80) |
| | | | (SEQ ID NO: 79) | |
| | | LC- | TCGGCATCCTACCGGTACAGT | SASYRYS |
| | | CDR2 | (SEQ ID NO: 81) | (SEQ ID NO: 82) |
| | | LC- | CAGCAATATAACAGCTATCCATTCACG | QQYNSYPFT |
| | | CDR3 | (SEQ ID NO: 83) | (SEQ ID NO: 84) |
| 14 | GD2/GD3 | HC- | GACTACAACATGGAC | DYNMD |
| Mouse | | CDR1 | (SEQ ID NO: 85) | (SEQ ID NO: 86) |
| IgM | | HC- | GATATTAATCCTAACAATGGTGGTACTA | DINPNNGGTIYNQKFKG |
| Kappa | | CDR2 | TCTACAACCAGAAGTTCAAGGGC | (SEQ ID NO: 88) |
| | | | (SEQ ID NO: 87) | |
| | | HC- | TCGGGGATCTACTATGATTACGCCTGGT | SGIYYDYAWFPY |
| | | CDR3 | TTCCTTAC | (SEQ ID NO: 90) |
| | | | (SEQ ID NO: 89) | |
| | | LC- | AGTGCAAGTCAGGGCATTAGCAATTATT | SASQGISNYLN |
| | | CDR1 | TAAAC | (SEQ ID NO: 92) |
| | | | (SEQ ID NO: 91) | |
| | | LC- | TACACATCAAGTTTACACTCA | YTSSLHS |
| | | CDR2 | (SEQ ID NO: 93) | (SEQ ID NO: 94) |
| | | LC- | CAGCAGTATAGTAAGCTTCCTCCTACG | QQYSKLPPT |
| | | CDR3 | (SEQ ID NO: 95) | (SEQ ID NO: 96) |
| 15 | GD2/GD3 | HC- | GGATTCACTTTTAGTGACGCCTGG | GFTFSDAW |
| Mouse | | CDR1 | (SEQ ID NO: 97) | (SEQ ID NO: 98) |
| mAb | | HC- | ATTAGAAACAAAGCTAATAATCATGCG | IRNKANNHAT |
| | | CDR2 | ACA | (SEQ ID NO: 100) |
| | | | (SEQ ID NO: 99) | |
| | | HC- | ACCGGGCGACATGATTCCTACTTTGACT | TGRHDSYFDY |
| | | CDR3 | AC | (SEQ ID NO: 102) |
| | | | (SEQ ID NO: 101) | |
| | | LC- | CAGGGCATTAGCAATTAT | QGISNY |
| | | CDR1 | (SEQ ID NO: 103) | (SEQ ID NO: 104) |
| | | LC- | TACACATCA | YTS |
| | | CDR2 | (SEQ ID NO: 105) | (SEQ ID NO: 106) |
| | | LC- | CAGCAGTATAGTAAGCTTCCTCCTACG | QQYSKLPPT |
| | | CDR3 | (SEQ ID NO: 107) | (SEQ ID NO: 108) |
| 17 | GD2/GD3 | HC- | GGCTACACCTTCACCAGCTACTGG | GYTFTSYW |
| Mouse | | CDR1 | (SEQ ID NO: 109) | (SEQ ID NO: 110) |
| mAb | | HC- | ATTTATCCTGGTAGTGGTAGTACT | IYPGSGST |
| | | CDR2 | (SEQ ID NO: 111) | (SEQ ID NO: 112) |
| | | HC- | GCAAGCCACCGATTTGATTACTACGGTA | ASHRFDYYGSSYYAMDY |
| | | CDR3 | GTAGCTACTATGCTATGGACTAC | (SEQ ID NO: 114) |
| | | | (SEQ ID NO: 113) | |
| | | LC- | CAGGACATTTGCAATTAT | QDICNY |
| | | CDR1 | (SEQ ID NO: 115) | (SEQ ID NO: 116) |
| | | LC- | TACACATCA | YTS |
| | | CDR2 | (SEQ ID NO: 117) | (SEQ ID NO: 118) |
| | | LC- | CAACAGGGTAATACGCTTCCGCTCACG | QQGNTLPLT |
| | | CDR3 | (SEQ ID NO: 119) | (SEQ ID NO: 120) |
| 18 | GD2/GD3 | HC- | GGATACACGTTCACTGACTTCCAC | GYTFTDFH |
| Mouse | | CDR1 | (SEQ ID NO: 121) | (SEQ ID NO: 122) |
| mAb | | HC- | ATTAATCCTAACAATGGTGGTACT | INPNNGGT |
| | | CDR2 | (SEQ ID NO: 123) | (SEQ ID NO: 124) |
| | | HC- | GTAAGAGAAATCTACTTTGGCTTTGACT | VREIYFGFDY |
| | | CDR3 | AC | (SEQ ID NO: 126) |
| | | | (SEQ ID NO: 125) | |
| | | LC- | CAGGACATTTGCAATTAT | QDICNY |
| | | CDR1 | (SEQ ID NO: 127) | (SEQ ID NO: 128) |
| | | LC- | TACACATCA | YTS |
| | | CDR2 | (SEQ ID NO: 129) | (SEQ ID NO: 130) |
| | | LC- | CAACAGGGTAATACGCTTCCGCTCACG | QQGNTLPLT |
| | | CDR3 | (SEQ ID NO: 131) | (SEQ ID NO: 132) |
| 19 | GD2 | HC- | GGATACACATTCACTAAATACACC | GYTFTKYT |
| Mouse | | CDR1 | (SEQ ID NO: 133) | (SEQ ID NO: 134) |
| mAb | | HC- | ATTAATCCTAACAATGGTGGTACT | INPNNGGT |
| | | CDR2 | (SEQ ID NO: 135) | (SEQ ID NO: 136) |
| | | HC- | ACAAGCAAGTCCTTTGACTAC | TSKSFDY |
| | | CDR3 | (SEQ ID NO: 137) | (SEQ ID NO: 138) |
| | | LC- | TCAAGTGTAAGTAAC | SSVSN |
| | | CDR1 | (SEQ ID NO: 139) | (SEQ ID NO: 140) |
| | | LC- | AGCACATCC | STS |
| | | CDR2 | (SEQ ID NO: 141) | (SEQ ID NO: 142) |
| | | LC- | CAACAAAGGAGTGGTTACCCATTCACG | QQRSGYPFT |
| | | CDR3 | (SEQ ID NO: 143) | (SEQ ID NO: 144) |

TABLE 2
Exemplary sequence of the leader and variable regions of anti-ganglioside
monoclonal antibodies

SEQ ID NO: 145 Clone 4 Heavy Chain Variable (vH) cDNA Sequence
CAAGTACAGCTGGAGGAGTCTGGGACTGTGCTGGCAAGGCCTGGGGCTTCAGTGAAGATGTCCTGCAAGACTT
CTGGCTACACATTTACCAGGTACTGGATGCACTGGGTAAAACAGAGGCCTGGACAGGGTCTGGAATGGATAGG
GGCTATTTATCCTGGAAATAGTGATACTACCTACAACCAGAAGTTCAAGGGCAAGGCCAAACTGACTGCAGTC
ACATCCGCCACCAATGCCTACATGGAAGTAAGCAGCCTGACAAATGAGGACTCTGCGGTCTATTACTGTGCAA
GATCCGATGGTCCTATGGACTACTGGGGTCAAGGAACCTCAGTCACCGTCTCCTCA
SEQ ID NO: 146 Clone 4 Heavy Chain Variable (vH) Amino Acid Sequence
QVQLEESGTVLARPGASVKMSCKTSGYTFTRYWMHWVKQRPGQGLEWIGAI YPGNSDTTYNQKFKGKAKLTAV
TSATNAYMEVSSLTNEDSAVYYCARSDGPMDYWGQGTSVTVSS
SEQ ID NO: 147 Clone 4 Light Chain (kappa) Variable (vL) cDNA Sequence
GACATTGTACTGACCCAATCTCCAGCTTCTTTGGCTGTGTCTCTAGGGCAGAGGGCCACCATTTCCTGCAGAG
CCAGCGAAAGTGTTGATAATTATGGCATCAGTTTTATGAACTGGTTCCAACAGAAACCAGGACAGCCACCCAA
ACTCCTCATCTATGCTGCATCCAATCAAGGATCCGGGGTCCCTGCCAGGTTTAGTGGCAGTGGGTCTGGGACA
GACTTCAGTCTCAACATCCATCCTATGGAGGAGGATGATACTGCAATGTATTTCTGTCAGCAAAGTAAGGAGG
TTCCGTTCACGTTCGGAGGGGGGACCAAGCTGGA
SEQ ID NO: 148 Clone 4 Light Chain (kappa) Variable (vL) Amino Acid
Sequence
DIVLTQSPASLAVSLGQRATISCRASESVDNYGISFMNWFQQKPGQPPKLLIYAASNQGSGVPARFSGSGSGT
DFSLNIHPMEEDDTAMYFCQQSKEVPFTFGGGTKL
SEQ ID NO: 149 Clone 6 Heavy Chain Variable (vH) cDNA Sequence
CAGATCCAGTTGGTACAATCTGGACCTGAGCTGAAGAAGCCTGGAGAGACAGTCAAGATCTCCTGCAAGGCTT
CTGGATATTCCTTCACAACCTATGGAATGAGCTGGGTGAAACAGGCTCCAGGAAAGGGTTTAAAGTGGATGGG
CTGGATAAACACATATACTGGAGTGCCAACATATGGTGATGACTTCAAGGGACGGTTTGCCTTCTCTTTGGAA
ACCTCTACCAGCACTGCCTATTTGCAGATCAACAACCTCAAAAATGACGACACGGCTTCATATTTCTGTGCAA
GATGGTTACGCCACCATGCTATGGACTACTGGGGTCAAGGAACCTCAGTCACCGTCTCCTCA
SEQ ID NO: 150 Clone 6 Heavy Chain Variable (vH) Amino Acid Sequence
QIQLVQSGPELKKPGETVKISCKASGYSFTTYGMSWVKQAPGKGLKWM
GWINTYTGVPTYGDDFKGRFAFSLETSTSTAYLQINNLKNDDTASYFCARWLRHHAMDYWGQGTSVTVSS
SEQ ID NO: 151 Clone 6 Light Chain (kappa) Variable (vL) cDNA Sequence
AACATTGTAATGACCCAATCTCCCAAATCCATGTCCATGTCAGTAGGAGAGAGGGTCACCTTGACCTGCAAGG
CCAGTGAGAATGTGGTTACTTATGTTTCCTGGTATCAACAGAAACCAGAGCAGTCTCCTAAACTGCTGATATA
CGGGGCATCCAACCGGTACACTGGGGTCCCCGATCGCTTCACAGGCAGTGGATCTGCAACAGATTTCACTCTG
ACCATCAGCAGTGTGCAGGCTGAAGACCTTGCAGATTATCACTGTGGACAGGGTTACAGCTATCCGTACACGT
TCGGAGGGGGGACCAAGCTGGAAATAAAA
SEQ ID NO: 152 Clone 6 Light Chain (kappa) Variable (vL) Amino Acid
Sequence
NIVMTQSPKSMSMSVGERVTLTCKASENVVTYVSWYQQKPEQSPKLLIYGASNRYTGVPDRFTGSGSATDFTL
TISSVQAEDLADYHCGQGYSYPYTFGGGTKLEIK
SEQ ID NO: 153 Clone 7 Heavy Chain Variable (vH) cDNA Sequence
CAGGTTCAGCTGGAGCAGTCTGGAGGAGCCTTGGTGCAACCTGGAGGATCCATGAAACTCTCTTGTGCTGCCT
CTGGATTCACTTTTAGTGACGCCTGGATGGACTGGGTCCGCCAGTCTCCAGAAAAGGGGCTTGAGTGGGTTGC
TGAAATTAGAAACAAAGCTAATAATCATGCGACATACTATGCTGAGTCTGTGAAAGGGAGGTTCACCATCTCA
AGAGATGATTCCAAAAATAGTGTCTACCTGCAAATGAACAACTTAAGAGCTGAAGACACTGGCATTTATTATT
GTACCAGGCGACATGATTCCTACTTTGACTACTGGGGCCAAGGCACCACTCTCACAGTCTCCTCA
SEQ ID NO: 154 Clone 7 Heavy Chain Variable (vH) Amino Acid Sequence
QVQLEQSGGALVQPGGSMKLSCAASGFTFSDAWMDWVRQSPEKGLEWVAEIRNKANNHATYYAESVKGRFTIS
RDDSKNSVYLQMNNLRAEDTGIYYCTRRHDSYFDYWGQGTTLTVSS
SEQ ID NO: 155 Clone 7 Light Chain (kappa) Variable (vL) cDNA Sequence
GACATTGTGATGACCCAGTCTCACAAATTCATGTCCACATCAGTAGGAGACAGGGTCAGCATCACCTACAAGG
CCAGTCAGGATGTGGATACTGCTGTAGCCTGGTATCAACAGAAACCAGGGCAATCTCCTAAACTACTGATTTA
CTGGGCATCCACCCGGCACACTGGAGTCCCTGATCGCTTCACAGGCAGTGGATCTGGGACAGATTTCACTCTC
ACCATTAACAATGTGCAGTCTGAAGACTTGGCAGATTATTTCTGTCAGCAATATCGCAGCTATCCTCTCACGT
TCGGTGCTGGGACCAAGCTGGAACTGAAACGG
SEQ ID NO: 156 Clone 7 Light Chain (kappa) Variable (vL) Amino Acid
Sequence
DIVMTQSHKFMSTSVGDRVSITYKASQDVDTAVAWYQQKPGQSPKLLIYWASTRHTGVPDRFTGSGSGTDFTL
TINNVQSEDLADYFCQQYRSYPLTFGAGTKLELKR
SEQ ID NO: 157 Clone 8 Heavy Chain Variable (vH) cDNA Sequence
GAAGTAAAGCTGCAGGAGTCTGGGACTGAGCTGGCAAGGCCTGGGGCTTCAGTGAAGATGTCCTGCAAGACTT
CTGGCTACACATTTACCAGTTACTGGATGCACTGGGTAAAACAGAGGCCTGGACAGGGTCTGGAATGGATAGG
GGCTATTTATCCTGGAAAAAGTGGTACTACCTACAACCAGAAGTTCAAGGGCAAGGCCAAACTGACTGCAGTC
ACATCCGCCAGCACTGCCTACATGGAACTCAGCAGCCTGACAAATGAGGACTCTGCGGTCTATTACTGTACAA
GATCCGATGGTCCTATGGACTACTGGGGTCAAGGAACCTCAGTCACCGTCTCCTCA
SEQ ID NO: 158 Clone 8 Heavy Chain Variable (vH) Amino Acid Sequence
EVKLQESGTELARPGASVKMSCKTSGYTFTSYWMHWVKQRPGQGLEWIGAIYPGKSGTTYNQKFKGKAKLTAV
TSASTAYMELSSLTNEDSAVYYCTRSDGPMDYWGQGTSVTVSS
SEQ ID NO: 159 Clone 8 Light Chain (kappa) Variable (vL) cDNA Sequence
GACATTGTGCTGACCCAATCTCCAGCTTCTTTGGCTGTGTCTCTAGGGCAGAGGGCCACCATCTCCTGCAGAG
CCAGCGAGAGTGTTGATAATTATGACATTAGTTTTATGAACTGGTTCCAACAGAAACCAGGACAGCCACCCAA
ACTCCTCATCTATGCTGCATCCAACCAAGGATCCGGGGTCCCTGCCAGGTTCAGTGGCAGTGGGTCTGGGACA
GACTTCAGTCTCAACATCCATCCTATGGAGGAGGATGATACTGCAATGTATTTCTGTCAGCAAAGTAAGGAGG
TTCCGTACACGTTCGGAGGGGGGACCAAGCTGGAAATAAAACGG
SEQ ID NO: 160 Clone 8 Light Chain (kappa) Variable (vL) Amino Acid
Sequence
DIVLTQSPASLAVSLGQRATISCRASESVDNYDISFMNWFQQKPGQPPKLLIYAASNQGSGVPARFSGSGSGT
DFSLNIHPMEEDDTAMYFCQQSKEVPYTFGGGTKLEIKR
SEQ ID NO: 161 Clone 9 Heavy Chain Variable (vH) cDNA Sequence
GAGGTCCAGCTGCAACAGTCTGGACCTGAGCTGGTGAAGCCTGGGGCTTCAGTGAAGATACCCTGCAAGGCTT
CTGGATACACATTCACTGACTACAACATGGACTGGGTGAAACAGAGCCATGGAAAGAGCCTTGAGTGGATTGG
AGATATTAATCCTAACAATGGTGGTACTATCTACAACCAGAAGTTCAAGGGCAAGGCCACATTGACTGTAGAC
AAGTCCTCCAGCACAGCCTACATGGAGCTCCGCAGCCTGACATCTGAGGACACTGCAGTCTATTACTGTGCAA
GATCGGGGATCTACTATGATTACGCCTGGTTTCCTTACTGGGGCCAAGGGACTCTGGTCACTGTCTCTGCA
SEQ ID NO: 162 Clone 9 Heavy Chain Variable (vH) Amino Acid Sequence
EVQLQQSGPELVKPGASVKIPCKASGYTFTDYNMDWVKQSHGKSLEWIGDINPNNGGTIYNQKFKGKATLTVD
KSSSTAYMELRSLTSEDTAVYYCARSGIYYDYAWFPYWGQGTLVTVSA
SEQ ID NO: 163 Clone 9 Light Chain (kappa) Variable (vL) cDNA Sequence
GATATCCAGATGACACAGACTACATCCTCCCTGTCTGCCTCTCTGGGAGACAGAGTCACCATCAGTTGCAGTG
CAAGTCAGGGCATTAGCAATTATTTAAACTGGTATCAGCAGAAACCAGATGGAACTGTTAAACTCCTGATCTA
TTACACATCAAGTTTACACTCAGGAGTCCCATCAAGGTTCAGTGGCAGTGGGTCTGGGACAGATTATTCTCTC
ACCATCAGCAACCTGGAACCTGAAGATATTGCCACTTACTATTGTCAGCAGTATAGTAAGCTTCCTCCTACGT
TCGGTGCTGGGACCAAGCTGGAGCTGAAA
SEQ ID NO: 164 Clone 9 Light Chain (kappa) Variable (vL) Amino Acid
DIQMTQTTSSLSASLGDRVTISCSASQGISNYLNWYQQKPDGTVKLLIYYTSSLHSGVPSRFSGSGSGTDYSL
TISNLEPEDIATYYCQQYSKLPPTFGAGTKLELK
SEQ ID NO: 165 Clone 10 Heavy Chain Variable (vH) cDNA Sequence
GAGGTCCAGCTGCAACAGTCTGGACCTGAGCTGGTGAAGCCTGGGGCTTCAGTGAAGATACCCTGCAAGGCTT
CTGGATACACATTCACTGACTACAACATGGACTGGGTGAAACAGAGCCATGGAAAGAGCCTTGAGTGGATTGG
AGATATTAATCCTAACAATGGTGGTACTATCTACAACCAGAAGTTCAAGGGCAAGGCCACATTGACTGTAGAC
AAGTCCTCCAGCACAGCCTACATGGAGCTCCGCAGCCTGACATCTGAGGACACTGCAGTCTATTACTGTGCAA
GATCGGGGATCTACTATGATTACGCCTGGTTTCCTTACTGGGGCCAAGGGACTCTGGTCACTGTCTCTGCAG
SEQ ID NO: 166 Clone 10 Heavy Chain Variable (vH) Amino Acid Sequence
EVQLQQSGPELVKPGASVKIPCKASGYTFTDYNMDWVKQSHGKSLEWIGDINPNNGGTIYNQKFKGKATLTVD
KSSSTAYMELRSLTSEDTAVYYCARSGIYYDYAWFPYWGQGTLVTVSA
SEQ ID NO: 167 Clone 10 Light Chain (kappa) Variable (vL) cDNA Sequence
GATATCCAGATGACACAGACTACATCCTCCCTGTCTGCCTCTCTGGGAGACAGAGTCACCATCAGTTGCAGTG
CAAGTCAGGGCATTAGCAATTATTTAAACTGGTATCAGCAGAAACCAGATGGAACTGTTAAACTCCTGATCTA
TTACACATCAAGTTTACACTCAGGAGTCCCATCAAGGTTCAGTGGCAGTGGGTCTGGGACAGATTATTCTCTC
ACCATCAGCAACCTGGAACCTGAAGATATTGCCACTTACTATTGTCAGCAGTATAGTAAGCTTCCTCCTACGT
TCGGTGCTGGGACCAAGCTGGAGCTGAAAC
SEQ ID NO: 168 Clone 10 Light Chain (kappa) Variable (vL) Amino Acid
DIQMTQTTSSLSASLGDRVTISCSASQGISNYLNWYQQKPDGTVKLLIYYTSSLHSGVPSRFSGSGSGTDYSL
TISNLEPEDIATYYCQQYSKLPPTFGAGTKLELK
SEQ ID NO: 169 Clone 13 Heavy Chain Variable (vH) cDNA Sequence
CAGGTTCAACTGCAGCAGTCTGGGGCTGAGCTGGTGAGGCCTGGGGCTTCAGTGACGCTGTCCTGCAAGGCTT
CGGGCTACACATTTACTGACTATGAAATGCACTGGGTGAAGCAGACACCTGTGCATGGCCTGGAATGGATTGG
AGCTATTGATCCTGAAACTGGTGGTACTGCCTACAATCAGAAGTTCAAGGGCAAGGCCATACTGACTGCAGAC
AAATCCTCCAGCACAGCCTACATGGAGCTCCGCAGCCTGACATCTGAGGACTCTGCCGTCTATTACTGTACAA
GAAGCTGGGACGGAGACTACTGGGGCCAAGGCACCACTCTCACAGTCTCCTCA
SEQ ID NO: 170 Clone 13 Heavy Chain Variable (vH) Amino Acid Sequence
QVQLQQSGAELVRPGASVTLSCKASGYTFTDYEMHWVKQTPVHGLEWIGAIDPETGGTAYNQKFKGKAILTAD
KSSSTAYMELRSLTSEDSAVYYCTRSWDGDYWGQGTTLTVSS
SEQ ID NO: 171 Clone 13 Light Chain (kappa) Variable (vL) cDNA Sequence
GACATTGTGATGACCCAGTCTCAAAAATTCATGTCCACATCAGTAGGAGACAGGGTCAGCGTCACCTGCAAGG
CCAGTCAGAATGTGGGTACTAATGTAGCCTGGTATCAACAGAAACCAGGGCAATCTCCTAAAGCACTGATTTA
CTCGGCATCCTACCGGTACAGTGGAGTCCCTGATCGCTTCACAGGCAGTGGATCTGGGACAGATTTCACTCTC
ACCATCAGCAATGTGCAGTCTGAAGACTTGGCAGAGTATTTCTGTCAGCAATATAACAGCTATCCATTCACGT
TCGGCTCGGGGACAAAGTTGGAAATAAAA
SEQ ID NO: 172 Clone 13 Light Chain (kappa) Variable (vL) Amino Acid
Sequence
DIVMTQSQKFMSTSVGDRVSVTCKASQNVGTNVAWYQQKPGQSPKALIYSASYRYSGVPDRFTGSGSGTDFTL
TISNVQSEDLAEYFCQQYNSYPFTFGSGTKLEIK
SEQ ID NO: 173 Clone 14 Heavy Chain Variable (vH) cDNA Sequence
GAGGTCCAGCTGCAACAGTCTGGACCTGAGCTGGTGAAGCCTGGGGCTTCAGTGAAGATACCCTGCAAGGCTT
CTGGATACACATTCACTGACTACAACATGGACTGGGTGAAACAGAGCCATGGAAAGAGCCTTGAGTGGATTGG
AGATATTAATCCTAACAATGGTGGTACTATCTACAACCAGAAGTTCAAGGGCAAGGCCACATTGACTGTAGAC
AAGTCCTCCAGCACAGCCTACATGGAGCTCCGCAGCCTGACATCTGAGGACACTGCAGTCTATTACTGTGCAA
GATCGGGGATCTACTATGATTACGCCTGGTTTCCTTACTGGGGCCAAGGGACTCTGGTCACTGTCTCTGCAG
SEQ ID NO: 174 Clone 14 Heavy Chain Variable (vH) Amino Acid Sequence
EVQLQQSGPELVKPGASVKIPCKASGYTFTDYNMDWVKQSHGKSLEWIGDINPNNGGTIYNQKFKGKATLTVD
KSSSTAYMELRSLTSEDTAVYYCARSGIYYDYAWFPYWGQGTLVTVSA
SEQ ID NO: 175 Clone 14 Light Chain (kappa) Variable (vL) cDNA Sequence
GATATCCAGATGACACAGACTACATCCTCCCTGTCTGCCTCTCTGGGAGACAGAGTCACCATCAGTTGCAGTG
CAAGTCAGGGCATTAGCAATTATTTAAACTGGTATCAGCAGAAACCAGATGGAACTGTTAAACTCCTGATCTA
TTACACATCAAGTTTACACTCAGGAGTCCCATCAAGGTTCAGTGGCAGTGGGTCTGGGACAGATTATTCTCTC
ACCATCAGCAACCTGGAACCTGAAGATATTGCCACTTACTATTGTCAGCAGTATAGTAAGCTTCCTCCTACGT
TCGGTGCTGGGACCAAGCTGGAGCTGAAAC
SEQ ID NO: 176 Clone 14 Light Chain (kappa) Variable (vL) Amino Acid
Sequence
DIQMTQTTSSLSASLGDRVTISCSASQGISNYLNWYQQKPDGTVKLLIYYTSSLHSGVPSRFSGSGSGTDYSL
TISNLEPEDIATYYCQQYSKLPPTFGAGTKLELK
SEQ ID NO: 177 Clone 15 Heavy Chain Variable (vH) cDNA Sequence
CAAGTTCAGCTGCAGGAGTCTGGAGGAGCCTTGGTGCAACCTGGAGGATCCATGAAACTCTCTTGTGCTGCCT
CTGGATTCACTTTTAGTGACGCCTGGATGGACTGGGTCCGCCAGTCTCCAGAAAAGGGGCTTGAGTGGGTTGC
TGAAATTAGAAACAAAGCTAATAATCATGCGACATACTATGCTGAGTCTGTGAAAGGGAGGTTCACCATCTCA
AGAGATGATTCCAAAAATAGTGTCTACCTGCAAATGAACAACTTAAGAGCTGAGGACACTGGCATTTATTACT
GTACCGGGCGACATGATTCCTACTTTGACTACTGGGGCCAAGGCACCACTCTCACAGTCTCCTCA
SEQ ID NO: 178 Clone 15 Heavy Chain Variable (vH) Amino Acid Sequence
QVQLQESGGALVQPGGSMKLSCAASGFTFSDAWMDWVRQSPEKGLEWVAEIRNKANNHATYYAESVKGRFTIS
RDDSKNSVYLQMNNLRAEDTGIYYCTGRHDSYFDYWGQGTTLTVSS
SEQ ID NO: 179 Clone 15 Light Chain (kappa) Variable (vL) cDNA Sequence
GATATCCAGATGACACAGACTACATCCTCCCTGTCTGCCTCTCTGGGAGACAGAGTCACCATCAGTTGCAGTG
CAAGTCAGGGCATTAGCAATTATTTAAACTGGTATCAGCAGAAACCAGATGGAACTGTTAAACTCCTGATCTA
TTACACATCAAGTTTACACTCAGGAGTCCCATCAAGGTTCAGTGGCAGTGGGTCTGGGACAGATTATTCTCTC
ACCATCAGCAACCTGGAACCTGAAGATATTGCCACTTACTATTGTCAGCAGTATAGTAAGCTTCCTCCTACGT
TCGGTGCTGGGACCAAGCTGGAGCTGAAACGG
SEQ ID NO: 180 Clone 15 Light Chain (kappa) Variable (vL) Amino Acid
Sequence
DIQMTQTTSSLSASLGDRVTISCSASQGISNYLNWYQQKPDGTVKLLIYYTSSLHSGVPSRFSGSGSGTDYSL
TISNLEPEDIATYYCQQYSKLPPTFGAGTKLELKR
SEQ ID NO: 181 Clone 17 Heavy Chain Variable (vH) cDNA Sequence
GAGGTCCAGCTGGAGGAGTCTGGGGCTGAGCTTGTGAAGCCTGGGGCTTCGGTGAAGATGTCCTGTAAGGCTT
CTGGCTACACCTTCACCAGCTACTGGATAACCTGGGTGAAGCAGAGGCCTGGACAAGGCCTTGAGTGGATTGG
AGATATTTATCCTGGTAGTGGTAGTACTAACTACAATGAGAAGTTCAAGAGCAAGGCCACACTGACTGTAGAC
ACATCCTCCAGCACAACCTACATGCAGCTCAGCAGCCTGACATCTGAGGACTCTGCGGTCTATTACTGTGCAA
GCCACCGATTTGATTACTACGGTAGTAGCTACTATGCTATGGACTACTGGGGTCAAGGAACCTCAGTCACCGT
CTCCTCA
SEQ ID NO: 182 Clone 17 Heavy Chain Variable (vH) Amino Acid Sequence
EVQLEESGAELVKPGASVKMSCKASGYTFTSYWITWVKQRPGQGLEWIGDIYPGSGSTNYNEKFKSKATLTVD
TSSSTTYMQLSSLTSEDSAVYYCASHRFDYYGSSYYAMDYWGQGTSVTVSS
SEQ ID NO: 183 Clone 17 Light Chain (kappa) Variable (vL) cDNA Sequence
GATATCCAGATGACACAGACTACATCCTCCCTGTCTGCCTCTCTGGGAGACAGAGTCACCATCAGTTGCAGGG
CAAGTCAGGACATTTGCAATTATTTAAACTGGTATCAGCAGAAACCAGATGGACCTTTTAAACTCCTGATCTT
CTACACATCAAGATTACACTCAGGAGTCCCATCAAGGTTCAGTGGCAGTGGGTCTGGAACAGATTATTCTCTC
ACCATTAGCAACCTGGAGCAAGAAGATATTGCCACTTACTTTTGCCAACAGGGTAATACGCTTCCGCTCACGT
TCGGTGCTGGGACCAAGCTGGAGCTGAAACGG
SEQ ID NO: 184 Clone 17 Light Chain (kappa) Variable (vL) Amino Acid
Sequence
DIQMTQTTSSLSASLGDRVTISCRASQDICNYLNWYQQKPDGPFKLLIFYTSRLHSGVPSRFSGSGSGTDYSL
TISNLEQEDIATYFCQQGNTLPLTFGAGTKLELKR
SEQ ID NO: 185 Clone 18 Heavy Chain Variable (vH) cDNA Sequence
GAAGTACAGCTGGAGGAGTCTGGACCTGAGCTGGTGAAGCCTGGGACTTCAGTGAAGATATCCTGTAAGGCTT
CTGGATACACGTTCACTGACTTCCACATTAACTGGGTGAAACAGAGCCATGGAAAGAACCTTGAGTGGATTGG
AGATATTAATCCTAACAATGGTGGTACTAACTACAACCAGAAATTCAAGGGCAAGGCCACATTGATTGTTGAC
AAGTCTTCCAGCGCAGCCTACATGGAGCTCCGCAGCCTGACATCTGAGGACTCTGCAGTCTATTATTGTGTAA
GAGAAATCTACTTTGGCTTTGACTACTGGGGCCAAGGCACCACTCTCACAGTCTCCTCA
SEQ ID NO: 186 Clone 18 Heavy Chain Variable (vH) Amino Acid Sequence
EVQLEESGPELVKPGTSVKISCKASGYTFTDFHINWVKQSHGKNLEWIGDINPNNGGTNYNQKFKGKATLIVD
KSSSAAYMELRSLTSEDSAVYYCVREIYFGFDYWGQGTTLTVSS
SEQ ID NO: 187 Clone 18 Light Chain (kappa) Variable (vL) cDNA Sequence
GATATCCAGATGACACAGACTACATCCTCCCTGTCTGCCTCTCTGGGAGACAGAGTCACCATCAGTTGCAGGG
CAAGTCAGGACATTTGCAATTATTTAAACTGGTATCAGCAGAAACCAGATGGACCTTTTAAACTCCTGATCTT
CTACACATCAAGATTACACTCAGGAGTCCCATCAAGGTTCAGTGGCAGTGGGTCTGGAACAGATTATTCTCTC
ACCATTAGCAACCTGGAGCAAGAAGATATTGCCACTTACTTTTGCCAACAGGGTAATACGCTTCCGCTCACGT
TCGGTGCTGGGACCAAGCTGGAGCTGAAACGG
SEQ ID NO: 188 Clone 18 Light Chain (kappa) Variable (vL) Amino Acid
Sequence
DIQMTQTTSSLSASLGDRVTISCRASQDICNYLNWYQQKPDGPFKLLIFYTSRLHSGVPSRFSGSGSGTDYSL
TISNLEQEDIATYFCQQGNTLPLTFGAGTKLELKR
SEQ ID NO: 189 Clone 19 Heavy Chain Variable (vH) cDNA Sequence
GAAGTGAAGCTGGAGGAGTCTGGACCTGAGCTGGTGAAGCCTGGGGCTTCAGTGAAGATATCCTGCAAGACTT
CTGGATACACATTCACTAAATACACCATGCACTGGGTGAAGCAGAGCCATGGAAAGAGCCTTGAGTGGATTGG
AGATATTAATCCTAACAATGGTGGTACTAACTACAACCAGAAGTTCAAGGGCACGGCCACATTGACTGTACAC
AAGTCCTCCACCACAGCCTACATGGAGCTCCGCAGCCTGACATCTGAGGATTCTGCAGTCTATTACTGTACAA
GCAAGTCCTTTGACTACTGGGGCCAAGGCACCACTCTCACAGTCTCCTCA
SEQ ID NO: 190 Clone 19 Heavy Chain Variable (vH) Amino Acid Sequence
EVKLEESGPELVKPGASVKISCKTSGYTFTKYTMHWVKQSHGKSLEWIGDINPNNGGTNYNQKFKGTATLTVH
KSSTTAYMELRSLTSEDSAVYYCTSKSFDYWGQGTTLTVSS
SEQ ID NO: 191 Clone 19 Light Chain (kappa) Variable (vL) cDNA Sequence
CAAATTGTTCTCACCCAGTCTCCAGCAATCATGTCTGCATCTCCAGGGGAGAAGGTCACCATAACCTGCAGTG
CCAGCTCAAGTGTAAGTAACATACACTGGTTCCAGCAGAAGCCAGGCACTTTTCCCAAACTCTGGATTTATAG
CACATCCACCCTGGCTTCTGGAGTCCCTGGTCGCTTCAGTGGCAGTGGATCTGGGACCTCTTACTCTCTCACA
ATCAGCCGAATGGGGGCTGAAGATGCTGCCACTTATTACTGCCAACAAAGGAGTGGTTACCCATTCACGTTCG
GCTCGGGGACAAAGTTGGAAATAAAACGG
SEQ ID NO: 192 Clone 19 Light Chain (kappa) Variable (vL) Amino Acid
Sequence
QIVLTQSPAIMSASPGEKVTITCSASSSVSNIHWFQQKPGTFPKLWIY
STSTLASGVPGRFSGSGSGTSYSLTISRMGAEDAATYYCQQRSGYPFTFGSGTKLEIKR
*Included in Tables 1 and 2 are RNA nucleic acid molecules (e.g., thymidine replaced with uridine), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Tables 1 and 2, or a portion thereof. Such nucleic acid molecules can have a function of the full-length nucleic acid as described further herein.
*The antibodies presented in Tables 1 and 2 bind the carbohydrate domain of the ganglioside GD2 and GD3.

### Nucleic Acids, Vectors, and Recombinant Host Cells

A further object of the invention relates to nucleic acid sequences encoding monoclonal antibodies and fragments thereof, immunoglobulins, and polypeptides of the present invention.

For example, in certain embodiments, the present invention relates, in part, to a nucleic acid sequence encoding the vH domain or the vL domain of the antibodies or antigen-binding fragment thereof of the present disclosure.

Typically, said nucleic acid is a DNA or RNA molecule, which may be included in any suitable vector, such as a plasmid, cosmid, episome, artificial chromosome, phage or a viral vector.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Thus, a further object of the invention relates to a vector comprising a nucleic acid of the present invention.

Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cell can be used. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other representative examples of plasmids include replicating plasmids comprising an origin of replication, or integrative plasmids, such as for instance pUC, pcDNA, pBR, and the like. Representative examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv-positive cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A further object of the present invention relates to a cell which has been transfected, infected or transformed by a nucleic acid and/or a vector according to the invention. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed.”

The nucleic acids of the present invention may be used to produce a recombinant polypeptide of the invention in a suitable expression system. The term “expression system” means a host cell and compatible vector under suitable conditions, e.g. for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL 1662, hereinafter referred to as “YB2/0 cell”), and the like. The YB2/0 cell is preferred, since ADCC activity of chimeric or humanized antibodies is enhanced when expressed in this cell.

The present invention also relates to a method of producing a recombinant host cell expressing an antibody or a polypeptide of the invention according to the invention, said method comprising the steps consisting of (i) introducing in vitro or ex vivo a recombinant nucleic acid or a vector as described herein into a competent host cell, (ii) culturing in vitro or ex vivo the recombinant host cell obtained and (iii), optionally, selecting the cells which express and/or secrete said antibody or polypeptide. Such recombinant host cells can be used for the production of antibodies and polypeptides of the invention.

In another aspect, the present invention provides isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides of the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library. Preferably, the cDNA library comprises at least 80% full-length sequences, preferably, at least 85% or 90% full-length sequences, and, preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences. Optionally, polynucleotides of this invention will encode at least a portion of an antibody encoded by the polynucleotides described herein. The polynucleotides of this invention embrace nucleic acid sequences that can be employed for selective hybridization to a polynucleotide encoding an antibody of the present invention. See, e.g., Ausubel, supra; Colligan, supra, each entirely incorporated herein by reference.

### Methods of Producing Antibodies

Antibodies and fragments thereof, immunoglobulins, and polypeptides of the present invention may be produced by any technique known in the art, such as, without limitation, any chemical, biological, genetic or enzymatic technique, either alone or in combination.

Knowing the amino acid sequence of the desired sequence, one skilled in the art can readily produce said antibodies or polypeptides, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions. Alternatively, antibodies and other polypeptides of the present invention can be synthesized by recombinant DNA techniques as is well-known in the art. For example, these fragments can be obtained as DNA expression products after incorporation of DNA sequences encoding the desired (poly) peptide into expression vectors and introduction of such vectors into suitable eukaryotic or prokaryotic hosts that will express the desired polypeptide, from which they can be later isolated using well-known techniques.

In particular, the present invention further relates to a method of producing an antibody or a polypeptide of the invention, which method comprises the steps consisting of: (i) culturing a transformed host cell according to the invention under conditions suitable to allow expression of said antibody or polypeptide; and (ii) recovering the expressed antibody or polypeptide.

Antibodies and other polypeptides of the present invention are suitably separated from the culture medium by conventional immunoglobulin purification procedures such as, for example, protein A-Sepharose, hydroxylapatite chromatography, gel electrophoresis, dialysis, affinity chromatography, ammonium sulfate or ethanol precipitation, acid extraction, anion or cation exchange chromatography, phosphocellulose chromatography, hydrophobic interaction chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Chimeric antibodies (e.g., mouse-human chimeras or non-rodent-human chimeras) of the present invention can be produced by obtaining nucleic sequences encoding VL and VH domains as previously described, constructing a human chimeric antibody expression vector by inserting them into an expression vector for animal cell having genes encoding human antibody CH and human antibody CL, and expressing the coding sequence by introducing the expression vector into an animal cell. The CH domain of a human chimeric antibody can be any region which belongs to human immunoglobulin, such as the IgG class or a subclass thereof, such as IgG1, IgG2, IgG3 and IgG4. Similarly, the CL of a human chimeric antibody can be any region which belongs to Ig, such as the kappa class or lambda class. chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Patent Publication PCT/US86/02269; Akira et al. European Patent Application 184,187; Taniguchi, M. European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

In addition, humanized antibodies can be made according to standard protocols such as those disclosed in U.S. Pat. No. 5,565,332. In another embodiment, antibody chains or specific binding pair members can be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art, e.g., as described in U.S. Pat. Nos. 5,565,332, 5,871,907, or 5,733,743. Humanized antibodies of the present invention can be produced by obtaining nucleic acid sequences encoding CDR domains, as previously described, constructing a humanized antibody expression vector by inserting them into an expression vector for animal cell having genes encoding (i) a heavy chain constant region identical to that of a human antibody and (ii) a light chain constant region identical to that of a human antibody, and expressing the genes by introducing the expression vector into an animal cell. The humanized antibody expression vector may be either of a type in which a gene encoding an antibody heavy chain and a gene encoding an antibody light chain exists on separate vectors or of a type in which both genes exist on the same vector (tandem type).

Methods for producing humanized antibodies based on conventional recombinant DNA and gene transfection techniques are well-known in the art (See, e.g., Riechmann L. et al. 1988; Neuberger M S. et al. 1985). Antibodies can be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592, 106; EP 519,596; Padlan EA (1991); Studnicka G M et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576).

In addition, methods for producing antibody fragments are well-known. For example, Fab fragments of the present invention can be obtained by treating an antibody which specifically reacts with a ganglioside with a protease such as papain. Also, Fabs can be produced by inserting DNA encoding Fabs of the antibody into a vector for prokaryotic expression system, or for eukaryotic expression system, and introducing the vector into a procaryote or eucaryote (as appropriate) to express the Fabs.

Similarly, F(ab′) 2 fragments of the present invention can be obtained treating an antibody which specifically reacts with a ganglioside with a protease, pepsin. Also, the F(ab′) 2 fragment can be produced by binding Fab′ described below via a thioether bond or a disulfide bond.

Fab′ fragments of the present invention can be obtained treating F(ab′) 2 which specifically reacts with a ganglioside with a reducing agent, dithiothreitol. Also, the Fab′ fragments can be produced by inserting DNA encoding a Fab′ fragment of the antibody into an expression vector for prokaryote, or an expression vector for eukaryote, and introducing the vector into a prokaryote or eukaryote (as appropriate) to perform its expression.

In addition, scFvs of the present invention can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv. To generate a humanized scFv fragment, a well-known technology called CDR grafting may be used, which involves selecting the complementary determining regions (CDRs) from a donor scFv fragment, and grafting them onto a human scFv fragment framework of known three dimensional structure (see, e.g., WO98/45322; WO 87/02671; U.S. Pat. Nos. 5,859,205; 5,585,089; 4,816,567; EP0173494).

### Modification of Antibodies, Immunoglobulins, and Polypeptides

Amino acid sequence modification(s) of the antibodies described herein are contemplated. For example, it may be desirable to improve the binding affinity and/or other biological properties of the antibody. It is known that when a humanized antibody is produced by simply grafting only CDRs in VH and VL of an antibody derived from a non-human animal in FRs of the VH and VL of a human antibody, the antigen binding activity is reduced in comparison with that of the original antibody derived from a non-human animal. It is considered that several amino acid residues of the VH and VL of the non-human antibody, not only in CDRs but also in FRs, are directly or indirectly associated with the antigen binding activity. Hence, substitution of these amino acid residues with different amino acid residues derived from FRs of the VH and VL of the human antibody would reduce binding activity and can be corrected by replacing the amino acids with amino acid residues of the original antibody derived from a non-human animal.

Modifications and changes may be made in the structure of the antibodies of the present invention, and in the DNA sequences encoding them, and still obtain a functional molecule that encodes an antibody and polypeptide with desirable characteristics. For example, certain amino acids may be substituted by other amino acids in a protein structure without appreciable loss of activity. Since the interactive capacity and nature of a protein define the protein's biological functional activity, certain amino acid substitutions can be made in a protein sequence, and, of course, in its DNA encoding sequence, while nevertheless obtaining a protein with like properties. It is thus contemplated that various changes may be made in the antibodies sequences of the invention, or corresponding DNA sequences which encode said polypeptides, without appreciable loss of their biological activity.

In one embodiment, amino acid changes may be achieved by changing codons in the DNA sequence to encode conservative substitutions based on conservation of the genetic code. Specifically, there is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code (see genetic code chart above).

As described above, an important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (<RTI 3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well-known to those of skill in the art and include: arginine and lysine; glutamate and aspartate; serine and threonine; glutamine and asparagine; and valine, leucine and isoleucine.

Another type of amino acid modification of the antibody of the invention may be useful for altering the original glycosylation pattern of the antibody to, for example, increase stability. By “altering” is meant deleting one or more carbohydrate moieties found in the antibody, and/or adding one or more glycosylation sites that are not present in the antibody. Glycosylation of antibodies is typically N-linked. “N-linked” refers to the attachment of the carbohydrate moiety to the side chain of an asparagine residue. The tripeptide sequences asparagine-X-serine and asparagines-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the antibody is conveniently accomplished by altering the amino acid sequence such that it contains one or more of the above-described tripeptide sequences (for N-linked glycosylation sites). Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, orhydroxyproline, (e) aromatic residues such as those of phenylalanine, tyrosine, or tryptophan, or (f) the amide group of glutamine. For example, such methods are described in WO87/05330.

Similarly, removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Sojahr H. et al. (1987) and by Edge, A S. et al. (1981). Enzymatic cleavage of carbohydrate moieties on antibodies can be achieved by the use of a variety of endo- and exo-glycosidases as described by Thotakura, N R. et al. (1987).

Other modifications can involve the formation of immunoconjugates. For example, in one type of covalent modification, antibodies or proteins are covalently linked to one of a variety of non proteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Conjugation of antibodies or other proteins of the present invention with heterologous agents can be made using a variety of bifunctional protein coupling agents including but not limited to N-succinimidyl (2-pyridyldithio) propionate (SPDP), succinimidyl (N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6 diisocyanate), and bis-active fluorine compounds (such as 1,5-difluoro-2,4-dinitrobenzene). For example, carbon labeled 1-isothiocyanatobenzyl methyldiethylene triaminepentaacetic acid (MX-DTPA) is an exemplary chelating agent for conjugation of radionucleotide to the antibody (WO 94/11026).

In another aspect, the present invention features antibodies that specifically bind a ganglioside (e.g., GD2 or GD3) conjugated to a moiety that allows detection. Conjugated anti-ganglioside antibodies can be used diagnostically or prognostically to monitor the ganglioside levels (e.g., GD2 or GD3) in blood or tissues as part of a clinical testing procedure, e.g., to diagnose whether a patient has a cancer, to determine different stages of a cancer, to monitor the progression of a cancer, to determine the efficacy of a given treatment regimen or to select patients most likely to response to a cancer therapy (e.g., immunotherapy). Examples of detectable moieties include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin (PE); an example of a luminescent material includes luminol; examples of bioluminescent materials include luciferase, luciferin, and aequorin, and examples of suitable radioactive material include ¹²⁵I, ¹³¹I, ³⁵S, or ³H. As used herein, the term “labeled”, with regard to the antibody, is intended to encompass direct labeling of the antibody by coupling (i.e., physically linking) a detectable substance, such as a radioactive agent or a fluorophore (e.g. fluorescein isothiocyanate (FITC) or phycoerythrin (PE) or indocyanine (Cy5)) to the antibody, as well as indirect labeling of the antibody by reactivity with a detectable substance. For example, an antibody may be labeled with a nucleic acid sequence that may be amplified and detected, or an antisense oligonucleotide to reduce expression of a particular gene, such that expression can then be detected and measured.

Techniques for conjugating such therapeutic moiety to antibodies are well-known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243 56 (Alan R. Liss, Inc. 1985); Hellstrom et al., “Antibodies For Drug Delivery”, in Controlled Drug Delivery (2nd Ed.), Robinson et al. (eds.), pp. 623 53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475 506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303 16 (Academic Press 1985), and Thorpe et al., “The Preparation And Cytotoxic Properties Of Antibody-Toxin Conjugates”, Immunol. Rev., 62:119 58 (1982).

### Gangliosides

Gangliosides are glycosphingolipids comprising one or more sialic acids.

Glycosphingolipids contain a hydrophobic ceramide or sphingoid lipid tail, which is usually anchored to the outer leaflet of the plasma membrane. They also contain an oligosaccharide moiety and are classified according to this carbohydrate structure (ganglio, isoganglio, lacto etc.). Gangliosides are an important subclass of glycosphingolipids, because they contain negatively charged sialic acids (N-acetylneuraminic acid or N-glycolylneuraminic acid) linked to the lipooligosaccharide moiety. Gangliosides are named and classified according to the number of sialic acid residues attached (M for one, D for two, T for 3 and Q for 4) to the inner sugar moiety and according to their chromatographic mobility. The numbering of the gangliosides (5-x) is based on the number (x) of the inner sugar moieties (glucose, galactose or GalNAc) according to the original experimental classification of Svennerholm. Thus, if x is 4, the gangliosides are: GM1, GD1, GT1, x=3 for GM2, GD2, GT2, and x=2 for GM3, GD3, and GT3.

As presented herein, gangliosides are tumor biomarkers (for example, see Table 3). In some embodiments, a tumor-associated ganglioside is selected from GD2, GD3, GD1b, GT1b, fucosyl-GM1, GloboH, polysialic acid (PSA), GM2, GM3, sialyl-Lewis^{X}, sialyl-Lewis^{Y}, sialyl-Lewis^{A}, sialyl-Lewis^{B}, Lewis^{Y}, any portion thereof, and modified version thereof. In preferred embodiments, the ganglioside is GD2, GD3, GM2, or GT1b.

In some embodiments, gangliosides are modified. Modified gangliosides are well known in the art. Exemplary modified gangliosides are disclosed in, e.g., Patent Publication Nos. WO 2015/081438 and WO 2018/112669, the entire contents of which are incorporated herein by reference. A ganglioside may be a monomer. Alternatively, as exemplified in Patent Publication Nos. WO 2015/081438 and WO 2018/112669, two or more gangliosides may be covalently attached to a common core to form a multimer. In some embodiments, the multimer comprises 2, 3, 4, 5, 6, 7, 8, 9, 10, 11, 12, 13, 14, 15, 16, 17, 18, 19, or 20 gangliosides. In some embodiments, the multimer comprises 2 gangliosides. In some embodiments, the multimer comprises 4 gangliosides. In some embodiments, the common core comprises polyamidoamine (PAMAM).

Accordingly, as used herein, the term ganglioside may refer to a ganglioside, a tumor-associated ganglioside, a portion thereof, a modified version thereof, a monomer, or a multimer.

In some embodiments, a ganglioside has the structure: [(A)-(P)y-(L)z]x-M, wherein A is a ganglioside, or any portion thereof; P is a ring; y is 0 or 1; L is a linker; z is 0 or 1; x is an integer from 1 to 32; M is a core.

In some embodiments, a ganglioside has the structure (A)x-[(P)y-(L)z]-(M)b, wherein A is a ganglioside, or any portion thereof; x is an integer from 1 to 32; P is a ring; y is 0 or 1; L is a linker; z is an integer from 0 to 8; M is a core; and b is 0 or 1. In some embodiments, the ring, P, is a cycloalkyl, heterocyclyl, aryl, or heteroaryl group.

In some embodiments, the ring, P, is a cycloalkyl group, wherein the term “cycloalkyl” includes any monovalent saturated or unsaturated non-aromatic cyclic hydrocarbon group from three to eight carbons, unless otherwise specified, and is exemplified by cyclopropyl, cyclobutyl, cyclopentyl, cyclohexyl, cycloheptyl, bicycle heptyl, and the like. When the cycloalkyl group includes one carbon-carbon double bond or one carbon-carbon triple bond, the cycloalkyl group can be referred to as a “cycloalkenyl” or “cycloalkynyl” group respectively. Exemplary cycloalkenyl and cycloalkynyl groups include cyclopentenyl, cydohexenyl, cydohexynyl, and the like. The cycloalkyl groups of this invention can be optionally substituted with: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₋₂₀ alkoxy (e.g., C₁-6 alkoxy, such as perfluoroalkoxy); (4) C₁₋₆ alkylsulfinyl; (5) C₆₋₁₀ aryl; (6)amino; (7) C₁₋₆ alk-C₆₋₁₀ aryl; (8) azido; (9) C₃₋₈ cycloalkyl; (10) C₁₋₆ alk-C₃₋₈ cycloalkyl; (11)halo; (12) C₁₋₁₂ heterocyclyl (e.g., C₁₋₁₂ heteroaryl); (13) (C₁₋₁₂ heterocyclyl)oxy; (14) hydroxy; (15) nitro; (16) C₁₋₂₀ thioalkoxy (e.g., C₁₋₆ thioalkoxy); (17)-(CH₂)_{q}CO₂R^{A}, where q is an integer from zero to four, and R^{A} is selected from the group consisting of (a)alkyl, (b) C₆₋₁₀ aryl, (c) hydrogen, and (d)alk-C₆₋₁₀ aryl; (18)-(CH₂)_{q}CONR^{B} R^{c}, where q is an integer from zero to four and where R^{B} and R^{C} are independently selected from the group consisting of (a) hydrogen, (b) C₆₋₁₀ alkyl, (c) C₆₋₁₀ aryl, and (d) C₁₋₆ alk-C₆₋₁₀ aryl; (19)-(CH₂)_{q}SO₂R^{D}, where q is an integer from zero to four and where R^{D} is selected from the group consisting of (a) C₆₋₁₀ alkyl, (b) C₆₋₁₀ aryl, and (c) C₁₋₆ alk-C₆₋₁₀ aryl; (20)-(CH₂)_{q}SO₂NR^{E} R^{F}, where q is an integer from zero to four and where each of R^{E} and R^{F} is, independently, selected from the group consisting of (a) hydrogen, (b) C₆₋₁₀ alkyl, (c) C₆₋₁₀ aryl, and (d) C₁₋₆ alk-C₆₋₁₀ aryl; (21)thiol; (22) C₆₋₁₀ aryloxy; (23) C₃₋₈ cycloalkoxy; (24) C₆₋₁₀ aryl-C₁₋₆ alkoxy; (25) C₁₋₆ alk-C₁₋₁₂ heterocyclyl (e.g., C₁₋₆ alk-C₁₋₁₂ heteroaryl); (26)oxo; (27) C₂₋₂₀ alkenyl; and (28) C₂₋₂₀ alkynyl. In some embodiments, each of these groups can be further substituted as described herein. For example, the alkylene group of a C₁-alkaryl or a C₁-alkheterocyclyl can be further substituted with an oxo group to afford the respective aryloyl and (heterocyclyl) oyl substituent group.

In some embodiments, the ring, P, is a heterocyclyl group, wherein the term “heterocyclyl” includes a 5-, 6- or 7-membered ring, unless otherwise specified, containing one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocyclyl groups are of 1 to 12 (e.g., 1 to 11, 1 to 10, 1 to 9, 2 to 12, 2 to 11, 2 to 10, or 2 to 9) carbons. The term “heterocyclyl” also represents a heterocyclic compound having a bridged multicyclic structure in which one or more carbons and/or heteroatoms bridges two non-adjacent members of a monocyclic ring, e.g., a quinuclidinyl group. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cydopentane ring, a cydopentene ring, or another monocyclic heterocyclic ring, such as indolyl, quinolyl, isoquinolyl, tetrahydroquinolyl, benzofuryl, benzothienyl and the like. Examples of fused heterocyclyls include tropanes and 1 2,3,5,8,8a-hexahydroindolizine. Heterocyclics include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidiniyl, morpholinyl, thiomorpholinyl, thiazolyl, thiazolidinyl, isothiazolyl, isothiazolidinyl, indolyl, indazolyl, quinolyl, isoquinolyl, quinoxalinyl, dihydroquinoxalinyl, quinazolinyl, cinnolinyl, phthalazinyl, benzimidazolyl, benzothiazolyl, benzoxazolyl, benzothiadiazolyl, furyl, thienyl, thiazolidinyl, isothiazolyl, triazolyl, tetrazolyl, oxadiazolyl (e.g., 1,2,3-oxadiazolyl), purinyl, thiadiazolyl (e.g., 1,2,3-thiadiazolyl), tetrahydrofuranyl, dihydrofuranyl, tetrahydrothienyl, dihydrothienyl, dihydroindolyl, dihydroquinolyl, tetrahydroquinolyl, tetrahydroisoquinolyl, dihydroisoquinolyl, pyranyl, dihydropyranyl, dithiazolyl, benzofuranyl, isobenzofuranyl, benzothienyl, and the like, including dihydro and tetrahydro forms thereof, where one or more double bonds are reduced and replaced with hydrogens. Still other exemplary heterocyclyls include: 2,3,4,5-tetrahydro-2-oxo-oxazolyl; 2,3-dihydro-2-oxo-1H-imidazolyl; 2,3,4,5-tetrahydro-5-oxo-1H-pyrazolyl (e.g., 2,3,4,5-tetrahydro-2-phenyl-5-oxo-1H-pyrazolyl); 2,3,4,5-tetrahydro-2,4-dioxo-1H-imidazolyl (e.g., 2,3,4,5-tetrahydro-2,4-dioxo-5-methyl-5-phenyl-1H-imidazolyl); 2,3-dihydro-2-thioxo-1,3,4-oxadiazolyl (e.g., 2,3-dihydro-2-thioxo-5-phenyl-1,3,4-oxadiazolyl); 4,5-dihydro-5-oxo-1H-triazolyl (e.g., 4,5-dihydro-3-methyl-4-amino 5-oxo-1H-triazolyl); 1,2,3,4-tetrahydro-2,4-dioxopyridinyl (e.g., 1,2,3,4-tetrahydro-2,4-dioxo-3,3-diethylpyridinyl); 2,6-dioxo-piperidinyl (e.g., 2,6-dioxo-3-ethyl-3-phenylpiperidinyl); 1,6-dihydro-6-oxopyridiminyl; 1,6-dihydro-4-oxopyrimidinyl (e.g., 2-(methylthio)-1,6-dihydro-4-oxo-5-methylpyrimidin-1-yl); 1,2,3,4-tetrahydro-2,4-dioxopyrimidinyl (e.g., 1,2,3,4-tetrahydro-2,4-dioxo-3-ethylpyrimidinyl); 1,6-dihydro-6-oxo-pyridazinyl (e.g., 1,6-dihydro-6-oxo-3-ethylpyridazinyl); 1,6-dihydro-6-oxo-1,2,4-triazinyl (e.g., 1,6-dihydro-5-isopropyl-6-oxo-1,2,4-triazinyl); 2,3-dihydro-2-oxo-1H-indolyl (e.g., 3,3-dimethyl-2,3-dihydro-2-oxo-1H-indolyl and 2,3-dihydro-2-oxo-3,3′-spiropropane-1H-indol-1-yl); 1,3-dihydro-1-oxo-2H-iso-indolyl; 1,3-dihydro-1,3-dioxo-2H-iso-indolyl; 1H-benzopyrazolyl (e.g., 1-(ethoxycarbonyl)-1H-benzopyrazolyl); 2,3-dihydro-2-oxo-1H-benzimidazolyl (e.g., 3-ethyl-2,3-dihydro-2-oxo-1H-benzimidazolyl); 2,3-dihydro-2-oxo-benzoxazolyl (e.g., 5-chloro-2,3-dihydro-2-oxo-benzoxazolyl); 2-oxo-2H-benzopyranyl; 1,4-benzodioxanyl; 1,3-benzodioxanyl; 2,3-dihydro-3-oxo,4H-1,3-benzothiazinyl; 3,4-dihydro-4-oxo-3H-quinazolinyl (e.g., 2-methyl-3,4-dihydro-4-oxo-3H-quinazolinyl); 1,2,3,4-tetrahydro-2,4-dioxo-3H-quinazolyl (e.g., 1-ethyl-1,2,3,4-tetrahydro-2,4-dioxo-3H-quinazolyl); 1,2,3,6-tetrahydro-2,6-dioxo-7H-purinyl (e.g., 1,2,3,6-tetrahydro-1,3-dimethyl-2,6-dioxo-7 H-purinyl); 1,2,3,6-tetrahydro-2,6-dioxo-1 H-purinyl (e.g., 1,2,3,6-tetrahydro-3,7-dimethyl-2,6-dioxo-1 H-purinyl); 2-oxobenz[c,d]indolyl; 1,1-dioxo-2H-naphth[1,8-c,d]isothiazolyl; and 1,8-naphthylenedicarboxamido. Additional heterocyclics include 3,3a,4,5,6,6a-hexahydro-pyrrolo[3,4-b]pyrrol-(2H)-yl, and 2,5-diazabicyclo[2.2.1]heptan-2-yl, homopiperazinyl (or diazepanyl), tetrahydropyranyl, dithiazolyl, benzofuranyl, benzothienyl, oxepanyl, thiepanyl, azocanyl, oxecanyl, and thiocanyl. Any of the heterocyclyl groups mentioned herein may be optionally substituted with one, two, three, four or five substituents independently selected from the group consisting of: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₋₂₀ alkoxy (e.g., C₁₋₆ alkoxy, such as perfluoroalkoxy); (4) C₁₋₆ alkylsulfinyl; (5) C₆₋₁₀ aryl; (6)amino; (7) C₁₋₆ alk-C₆₋₁₀ aryl; (8) azido; (9) C₃₋₈ cycloalkyl; (10) C₁₋₆ alk-C₃₋₈ cycloalkyl; (11)halo; (12) C₁₋₁₂ heterocyclyl (e.g., C₁₋₁₂ heteroaryl); (13) (C₁₋₁₂ heterocyclyl)oxy; (14) hydroxy; (15) nitro; (16) C₁-20 thioalkoxy (e.g., C₁₋₆ thioalkoxy); (17)-(CH₂)_{q}CO₂R^{A}, where q is an integer from zero to four, and R^{A} is selected from the group consisting of (a) C₁₋₆ alkyl, (b) C₆₋₁₀ aryl, (c) hydrogen, and (d) C₁₋₆ alk-C₆₋₁₀ aryl; (18)-(CH₂)_{q}CONR^{B}′ R^{E}′, where q is an integer from zero to four and where R^{B}′ and R^{c}′ are independently selected from the group consisting of (a) hydrogen, (b) C₁₋₆ alkyl, (c) C₃₋₁₀ aryl, and (d) C₁₋₆ alk-C₆₋₁₀ aryl; (19)-(CH₂)_{q}SO₂R^{D}, where q is an integer from zero to four and where R^{D} is selected from the group consisting of (a)alkyl, (b) C₆₋₁₀ aryl, and (c)alk-C₆₋₁₀ aryl; (20)-(CH₂)_{q}SO₂NR^{E}′ R^{F}′, where q is an integer from zero to four and where each of R^{E}′ and R^{F}′ is, independently, selected from the group consisting of (a) hydrogen, (b) C₁₋₆ alkyl, (c) C₆₋₁₀ aryl, and (d) C₁₋₆ alk-C₆₋₁₀ aryl; (21)thiol; (22) C₆₋₁₀ aryloxy; (23) C₃₋₈ cycloalkoxy; (24) arylalkoxy; (25) C₁₋₆ alk-C₁₋₁₂ heterocyclyl (e.g., C₁₋₆ alk-C₁₋₁₂ heteroaryl); (26)oxo; (27) (C₁₋₁₂ heterocyclyl)imino; (28) C₂₋₂₀ alkenyl; (29) C₂₋₂₀ alkynyl; and (30)-(CH₂)_{q}CONR^{B}, where q is an integer from zero to four and where R^{B} is selected from the group consisting of (a) hydrogen, (b) C₁₋₆ alkyl, (c) C₃₋₁₀ aryl, and (d) C₁₋₆ alk-C₆₋₁₀ aryl. In some embodiments, each of these groups can be further substituted as described herein. For example, the alkylene group of a C₁-alkaryl or a C₁-alkheterocyclyl can be further substituted with an oxo group to afford the respective aryloyl and (heterocyclyl) oyl substituent group.

In some embodiments, the ring, P, is an aryl group. The term “aryl” includes a mono-, bicyclic, or multicyclic carbocyclic ring system having one or two aromatic rings and is exemplified by phenyl, naphthyl, 1,2-dihydronaphthyl, 1,2,3,4-tetrahydronaphthyl, anthracenyl, phenanthrenyl, fluorenyl, indanyl, indenyl, and the like, and may be optionally substituted with 1, 2, 3, 4, or 5 substituents. In some embodiments, the 1, 2, 3, 4, or 5 substituents are independently selected from the group consisting of: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₋₂₀ alkoxy (e.g., C₁₋₆ alkoxy, such as perfluoroalkoxy); (4) C₁₋₆ alkylsulfinyl; (5) C₆₋₁₀ aryl; (6)amino; (7) C₁₋₆ alk-C₆₋₁₀ aryl; (8) azido; (9) C₃₋₈ cycloalkyl; (10) C₁₋₆ alk-C₃-8 cycloalkyl; (11)halo; (12) C₁₋₁₂ heterocyclyl (e.g., C₁₋₁₂ heteroaryl); (13) (C₁₋₁₂ heterocyclyl)oxy; (14) hydroxy; (15) nitro; (16) C₁₋₂₀ thioalkoxy (e.g., C₁₋₆ thioalkoxy); (17) (CH₂)_{q}CO₂R^{A}, where q is an integer from zero to four, and R^{A} is selected from the group consisting of (a) C₁₋₆ alkyl, (b) C₆₋₁₀ aryl, (c) hydrogen, and (d) C₁₋₆ alk-C₆₋₁₀ aryl; (18)-(CH₂)_{q}CONR^{B}′ R^{C}′, where q is an integer from zero to four and where R^{B}′ and R^{c}′ are independently selected from the group consisting of (a) hydrogen, (b) C₁₋₆ alkyl, (c) C₃-10 aryl, and (d) C₁₋₆ alk-C₆₋₁₀ aryl; (19)-(CH₂)_{q}SO₂R^{D}, where q is an integer from zero to four and where R^{D} is selected from the group consisting of (a)alkyl, (b) C₆₋₁₀ aryl, and (c)alk-C₆₋₁₀ aryl; (20)-(CH₂)_{q}SO₂NR^{E}′ R^{F}′, where q is an integer from zero to four and where each of R^{E}′ and R^{F}′ is, independently, selected from the group consisting of (a) hydrogen, (b) C₁₋₆ alkyl, (c) C₆₋₁₀ aryl, and (d) C₁₋₆ alk-C₆₋₁₀ aryl; (21)thiol; (22) C₆₋₁₀ aryloxy; (23) C₃₋₈ cycloalkoxy; (24) C₆₋₁₀ aryl-C₁₋₆ alkoxy; (25) C₁₋₆ alk-C₁₋₁₂ heterocyclyl (e.g., C₁₋₆ alk-C₁₋₁₂ heteroaryl); (26) C₂₋₂₀ alkenyl; (27) C₂₋₂₀ alkynyl; and (28)-(CH₂)_{q}CONR^{B}, where q is an integer from zero to four and where R^{B} is selected from the group consisting of (a) hydrogen, (b) C₁₋₆ alkyl, (c) C₃₋₁₀ aryl, and (d) C₁₋₆ alk-C₆₋₁₀ aryl. In some embodiments, each of these groups can be further substituted as described herein. For example, the alkylene group of a C₁-alkaryl or a C₁alkheterocyclyl can be further substituted with an oxo group to afford the respective aryloyl and (heterocyclyl) oyl substituent group.

In some embodiments, the aryl is a heteroaryl group, wherein the “heteroaryl” includes a subset of heterocyclyls, as defined herein, which are aromatic: i.e., they contain 4n+2 pi electrons within the mono- or multicyclic ring system. Exemplary unsubstituted heteroaryl groups are of 1 to 12 (e.g., 1 to 11, 1 to 10, 1 to 9, 2 to 12, 2 to 11, 2 to 10, or 2 to 9) carbons. In some embodiment, the heteroaryl is substituted with 1, 2, 3, or 4 substituents groups as defined for a heterocyclyl group.

In some embodiments, the aryl is a C₃-C₁₀ aryl group.

In some embodiments, the aryl is a C₆-C₁₀ aryl group, such as phenyl or aminophenyl (e.g., p-aminophenyl).

In some embodiments, the aryl is selected from the group consisting of phenyl, hydroxyl-napthyl, naphthalene-2,6-diamine, 5-aminonaphthalen-1-ol, naphthalene-1,5-diol, naphthalene-1,5-diamine, (3-aminomethyl)cyclopentylamine, cyclopentane-1,3-diyldimethanamine, 3-(aminomethyl)cyclopentanamine, quinoline-3,7-diamine, 4-aminoquinolin-8-ol, quinoline-4,8-diol, quinoline-4,8-diamine, isoquinoline-4,8-diamine, pyridine-2,6-dicarboxylic acid, triazine, triazole, imidazole, morpholino, or 4-(aminomethyl) piperidine, any of which may be optionally substituted as defined for a heterocyclyl group.

In some embodiments, the linker, L, is a linkage between two elements (e.g., between the carbohydrate antigen and the core, or between the ring and the core). A linker can be a covalent bond (e.g., any bond created by chemical conjugation, such as an amide, ester, ether, azide, isothiocyanate, or disulfide bond) or a spacer (e.g., a moiety or amino acid sequence) that joins two elements and provides space and/or flexibility between the two elements. In some embodiments, the linker is a hydrocarbon linker (e.g., C₂-C₂₀ alkyl, C₂-C₂₀ alkenyl, or C₂-C₂₀ alkynyl), a polyamine linker (e.g, ethylene diamine, putrecine, cadaverine, spermidine, or spermine), a peptide linker (e.g, a 1, 2, 3, 4, 5, 6, 7, 8, 9, or 10 amino acid sequence), or a synthetic polymer (e.g., a polyether, such as polyethylene glycol).

In some embodiments, the core, M, is a moiety that contains one or more sites capable of being linked to carbohydrate antigen (e.g., either directly or indirectly through a linker and/or ring) wherein the linkage may be a covalent linkage (e.g., by a covalent bond) or a non-covalent linkage (e.g., via an affinity binding pair). A core may have, for example 1, 2, 3, 4, 5, 6, 7, 8, 9, 10, 12, 16, 20, 24, 28, 32 or more linkage sites.

In some embodiments, the core is amine. In some embodiments, the core is selected from the group consisting of branched polymers (e.g., star-shaped polymers, comb polymers, brush polymers, hyperbranched polymers, and dendrimers (e.g., poly (amidoamine) (PAMAM) dendrimers)); nucleic acids (e.g., oligonucleotides or longer nucleic acid molecules); polyamines (e.g. ethylene diamine or 2,4,6-tripyridyl-S-triazine); polypeptides (e.g., streptavidin and antibodies or antigen-binding fragments thereof, or carrier proteins (e.g., KLH)); polysaccharides (e.g., bacterial polysaccharides or plant polysaccharides), and micelles.

In some embodiments, the core, M, is a dendrimer, such as a poly (amidoamine) (PAMAM) dendrimer. In some embodiments, the core is a PAMAM dendrimer and “x” is 4 or more (e.g., 4, 5, 6, 7, 8, 9, 10, 12, 14, 16, 18, 20, 22, 24, 26, 28, 30, or 32 or more).

In some embodiments, the core is a carrier protein, such as keyhole limpet hemocyanin (KLH).

In some embodiment, the core is ethylene diamine and “x” is 2.

In some embodiments, the core is amine and “x” is 3.

In some embodiments, the core is 2,4,6-tripyridyl-S-triazine and “x” is 3.

In some embodiments, “x” is an integer from 1 to 32 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32). In preferred embodiments, “x” is 2, 3, 4, 8, or 16.

For example, in some embodiments, GD2 refers to a ganglioside having the following structure or a modified version thereof:

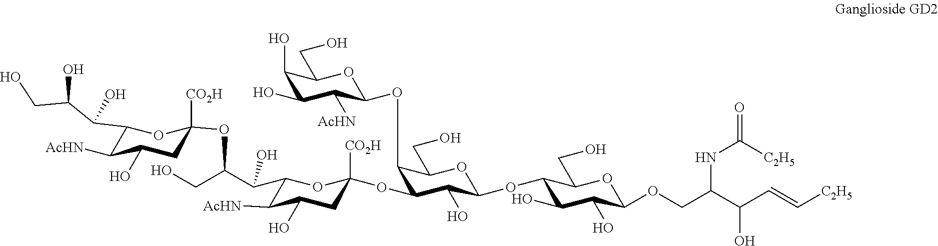

In some embodiments, GD2 comprises the following structure:

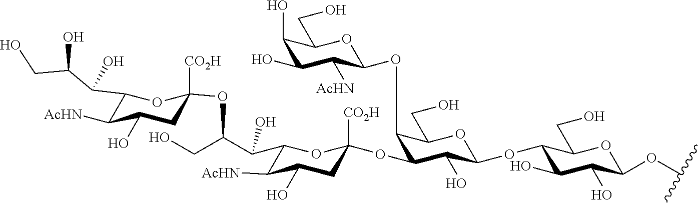

In some embodiments, GD2 comprises a thiophenyl group (e.g., thiophenyl GD2 as shown below).

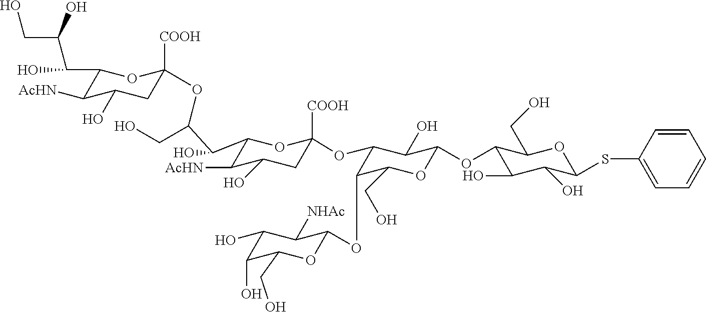 [SMILES: CC(=O)NC1C(OC2C(CO)OC(OC3C(CO)OC(Sc4ccccc4)C(O)C3O)C(O)C2OC2(C(=O)O)CC(O)C(NC(C)=O)C([C@H](O)C(CO)OC3(C(=O)O)CC(O)C(NC(C)=O)C([C@H](O)[C@H](O)CO)O3)O2)OC(CO)C(O)C1O]

In some embodiments, GD2 comprises an aryl group.

In some embodiments, GD2 comprises GD2-O-aryl-NH2 having the following structure:

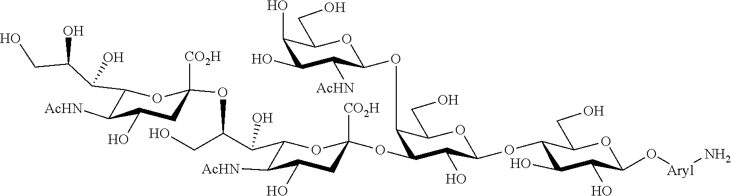

In some embodiments, GD2 comprises a p-amino phenyl ether group (e.g., AP-GD2 as shown below).

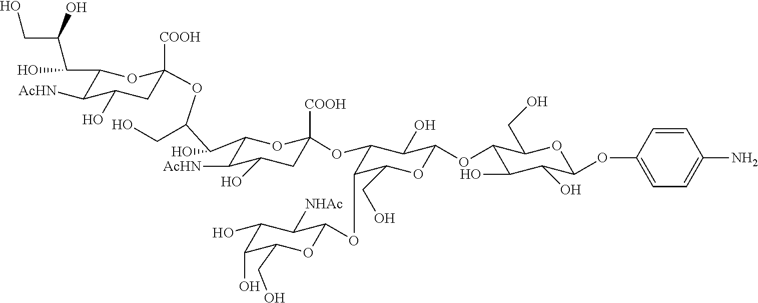 [SMILES: CC(=O)NC1C(OC2C(CO)OC(OC3C(CO)OC(Oc4ccc(N)cc4)C(O)C3O)C(O)C2OC2(C(=O)O)CC(O)C(NC(C)=O)C([C@H](O)C(CO)OC3(C(=O)O)CC(O)C(NC(C)=O)C([C@H](O)[C@H](O)CO)O3)O2)OC(CO)C(O)C1O]

Similarly, in some embodiments, GD3 refers to a ganglioside having the following structure or a modified version thereof:

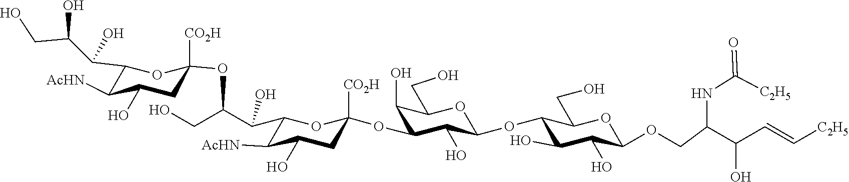

In some embodiments, GD3 comprises the following structure:

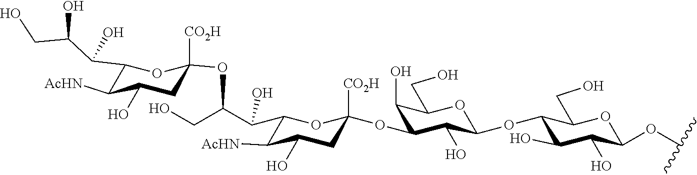

In some embodiments, GD3 is a thiophenyl GD3. In some embodiments, the thiophenyl GD3 has the following structure:

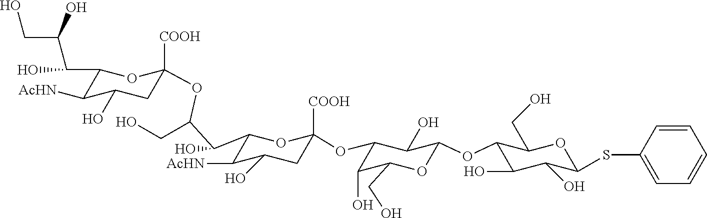 [SMILES: CC(=O)NC1C(O)CC(OC2C(O)C(CO)OC(OC3C(CO)OC(Sc4ccccc4)C(O)C3O)C2O)(C(=O)O)OC1[C@H](O)C(CO)OC1(C(=O)O)CC(O)C(NC(C)=O)C([C@H](O)[C@H](O)CO)O1]

In some embodiments, GD3 is GD3-O-aryl-NH2 having the following structure:

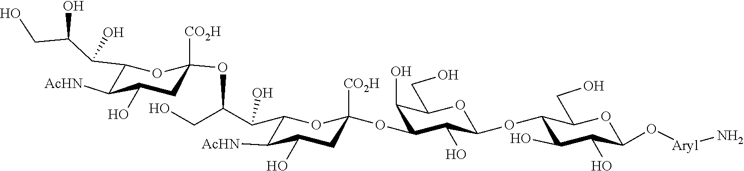

In some embodiments, GD3 is a p-amino phenyl ether GD3 (AP-GD3).

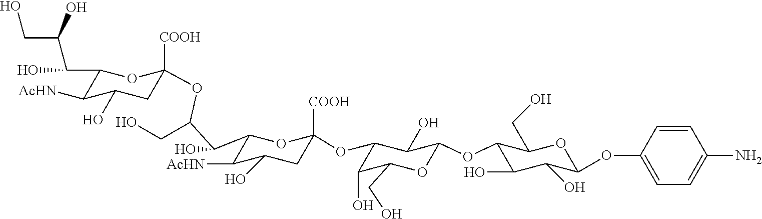 [SMILES: CC(=O)NC1C(O)CC(OC2C(O)C(CO)OC(OC3C(CO)OC(Oc4ccc(N)cc4)C(O)C3O)C2O)(C(=O)O)OC1[C@H](O)C(CO)OC1(C(=O)O)CC(O)C(NC(C)=O)C([C@H](O)[C@H](O)CO)O1]

In some embodiments, the aryl is a triazine. In some embodiments, the triazine is a 1, 3, 5-triazine. In some embodiments, the triazine is optionally substituted. In some embodiments, the triazine is 2-Amino-4,6-dichloro-1,3,5-triazine.

Accordingly, provided herein is a ganglioside comprising a heteroaryl, e.g., a triazine or a triazole. In some embodiments, the ganglioside comprising a heteroaryl is a monomer. In other embodiments, the ganglioside comprising a heteroaryl is a multimer (e.g., a trimer).

Thus, in certain aspects, provided herein is a composition comprising a ganglioside having the structure: (A)x-[(P)y-(L)z]-(M)b; wherein A is a ganglioside, or any portion thereof; x is an integer from 1 to 32; P is a heteroaryl; y is 1; L is a linker; z is an integer from 0 to 8; M is a core; and b is 0 or 1; wherein P is optionally substituted with 1, 2, 3, 4, or 5 substituents independently selected from (1) hydrogen; (2) C₁₋₇ acyl; (3) C₁₋₂₀ alkyl; (4)amino; (5) C₃₋₁₀ aryl; (6) hydroxy; (7) nitro; (8) C₁₋₂₀ alkyl-amino; and (9)-(CH₂)_{q}CONR^{B}, where q is an integer from 0 to 4 and where R^{B} is selected from the group consisting of (a) hydrogen, (b) C₁₋₆ alkyl, (c) C₃₋₁₀ aryl, and (d) C₁₋₆ alk-C₆₋₁₀ aryl.

In some embodiments, the heteroaryl is triazine or triazole. In some embodiments, a) the triazine is 1, 3, 5 triazine; or b) the triazole is 1, 2, 3 triazole or 1, 2, 4 triazole.

In some embodiments, the P is substituted with 1, 2, 3, 4, or 5 substituents independently selected from (1) hydrogen; (2) C₁₋₂₀ alkyl-amino; and (3)-(CH₂)_{q}CONR^{B}, where q is an integer from 0 to 4 and where R^{B} is selected from the group consisting of (a) hydrogen, (b) C₁₋₆ alkyl, (c) C₃₋₁₀ aryl, and (d) C₁₋₆ alk-C₆₋₁₀ aryl.

In some embodiments, the Mis (1)amine or (2) polyamidoamine (PAMAM). In some embodiments, x is 1, 2, 3, 4, 6, or 8.

Exemplary structures include:

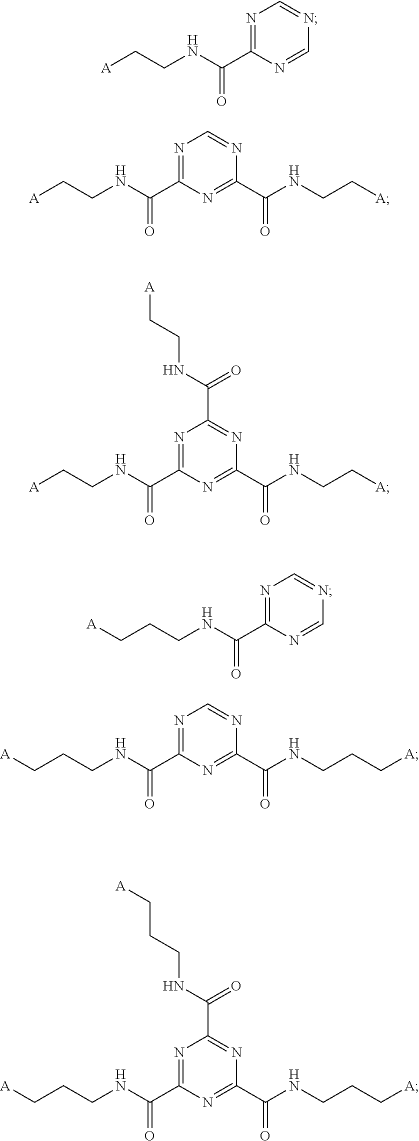

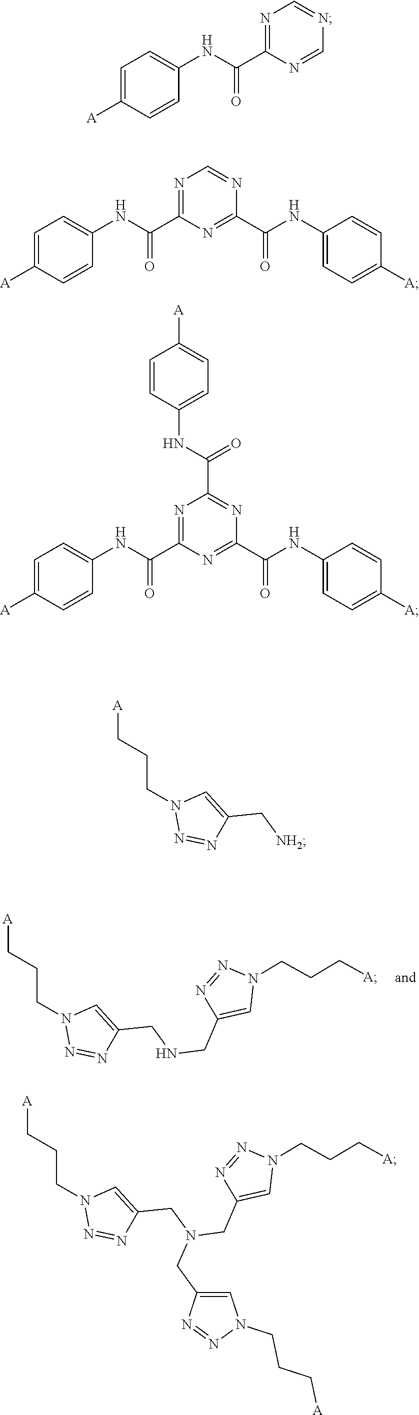

wherein A is a ganglioside.

In some embodiments, the ganglioside is selected from GD2, GD3, GD1b, GT1b, fucosyl-GM1, GloboH, polysialic acid (PSA), GM2, GM3, sialyl-Lewis^{X}, sialyl-Lewis^{Y}, sialyl-Lewis^{A}, sialyl-Lewis^{B}, and Lewis^{Y}, optionally wherein the ganglioside is GD2, GD3, GT1b, and GM2.

In some embodiments, the ganglioside is detectably labeled, optionally wherein the ganglioside is labeled with an enzyme, prosthetic group (e.g., streptavidin/biotin), fluorophore, luminescent tag, bioluminescent tag, and/or a radioisotope.

As demonstrated herein, the gangliosides of the present disclosure (natural or modified) can be labeled with a moiety that allows detection. Labeled gangliosides can be used as an antigen that can be used diagnostically or prognostically to monitor the level of anti-ganglioside antibodies in blood or tissues as part of a clinical testing procedure. The labeled gangliosides can also be used in various assays (e.g., ELISA assays, competitive ELISA assays, etc.). Examples of detectable moieties include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin (PE); an example of a luminescent material includes luminol; examples of bioluminescent materials include luciferase, luciferin, and aequorin, and examples of suitable radioactive material include ¹²⁵I, ¹³¹I, ³⁵S, or ³H. As used herein, the term “labeled”, with regard to the ganglioside, is intended to encompass direct labeling of the ganglioside by coupling (i.e., physically linking) a detectable substance, such as a radioactive agent or a fluorophore (e.g. fluorescein isothiocyanate (FITC) or phycoerythrin (PE) or indocyanine (Cy5)) to the ganglioside, as well as indirect labeling of the ganglioside by reactivity with a detectable substance. For example, a ganglioside may be labeled with a nucleic acid sequence that may be amplified and detected.

In some embodiments, the composition comprising the ganglioside of the present disclosure is a pharmaceutical composition.

### Pharmaceutical Compositions

Compounds that induce immune response against a ganglioside can be incorporated into pharmaceutical compositions suitable for administration to a subject. Such compositions typically comprise the compounds (e.g., modified forms of a ganglioside) and a pharmaceutically acceptable carrier. As used herein the pharmaceutically acceptable carrier is intended to include any and all solvents, dispersion media, coatings, antibacterial and antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary active compounds can also be incorporated into the compositions.

A pharmaceutical composition of the present invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition should be sterile and should be fluid to the extent that easy syringeability exists. It must be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Inhibition of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, the compounds of the present disclosure are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the present invention are dictated by, and directly dependent on, the unique characteristics of the active compound, the particular therapeutic effect to be achieved, and the limitations inherent in the art of compounding such an active compound for the treatment of individuals.

### Analyzing/Detecting Gangliosides

A ganglioside biomarker can be analyzed according to the methods described herein and other suitable techniques known in the art. The presence, level, or the lipid length of a ganglioside (e.g., GD1, GD2, GD3, GM2) can be detected using methods including, without limitation, immunodiffusion, immunoelectrophoresis, an immunofluorescence assay, an enzyme immunoassay, an immunoprecipitation assay, a chemiluminescence assay, an immunohistochemical assay, a dot blot assay, or a slot blot assay. General techniques to be used in performing the various immunoassays noted above and other variations of the techniques, such as in situ proximity ligation assay (PLA), fluorescence polarization immunoassay (FPIA), fluorescence immunoassay (FIA), enzyme immunoassay (EIA), nephelometric inhibition immunoassay (NIA), enzyme linked immunosorbent assay (ELISA), radioimmunoassay (RIA), sandwich ELISA, competitive ELISA, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference) alone or in combination or alternatively with NMR, MALDI-TOF, LC-MS/MS, are known to those of ordinary skill in the art.

Such reagents can also be used to monitor the ganglioside levels on a cell or tissue. Detection can be facilitated by coupling (e.g., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples pf suitable radioactive material include ¹²⁵I, ¹³¹I, ³⁵S, or ³H.

In some embodiments, ELISA and RIA procedures may be conducted such that a desired biomarker protein standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the biomarker protein in the sample is allowed to react with the corresponding immobilized antibody, radioisotope- or enzyme-labeled anti-biomarker protein antibody is allowed to react with the system, and radioactivity or the enzyme assayed. Other conventional methods may also be employed as suitable.

In some embodiments, sandwich ELISA is used to detect and measure the ganglioside levels. The sandwich ELISA quantify antigens between two layers of antibodies (i.e. capture and detection antibody). The antigen to be measured must contain at least two antigenic epitope capable of binding to antibody, since at least two antibodies act in the sandwich. Either monoclonal or polyclonal antibodies can be used as the capture and detection antibodies in Sandwich ELISA systems. Monoclonal antibodies recognize a single epitope that allows fine detection and quantification of small differences in antigen. A polyclonal is often used as the capture antibody to pull down as much of the antigen as possible. The advantage of Sandwich ELISA is that the sample does not have to be purified before analysis, and the assay can be very sensitive (up to 2 to 5 times more sensitive than direct or indirect ELISA).

In some embodiments, sandwich ELISA comprises use of one or more antibodies of the present disclosure (e.g., anti-GD2 or anti-GD3 antibody). In some embodiments, sandwich ELISA comprises use of one or more antibodies well known in the art (e.g., anti-GD2 or anti-GD3 antibody that is commercially available). In some embodiments, sandwich ELISA comprises use of a combination of an antibody of the present disclosure and an antibody that is known in the art (e.g., commercially available antibody).

A number of anti-GD2 or anti-GD3 antibodies suitable for the assays of the present disclosure (e.g., sandwich ELISA) are known in the art and/or commercially available.

Non-limiting examples of anti-GD2 antibodies include: murine m3F8 antibody, murine 14G2a antibody, dinutuximab, Ch14.18/CHO (Dinutuximab-beta), human 3F8 antibody, Naxitamab, and Hu14.18-K322A (see Sait and Modak (2017) Expert Rev Anticancer Ther. 17:889-904; Voeller and Sondel (2020) J Pediatr Hematol Oncol 41:163-169; Mora et al (2020) J of Clin Oncol 38:15); Ca.t No. BE0318 (BioXCell, Lebanon, NH); Cat. Nos. ab68456, ab82717 (Abcam, Waltham, MA); Cat. Nos. AGM-039YJ, AGM-143YJ, AGM-144YJ, AGM-145YJ, AGM-146YJ (Creative Biolabs, Shirley, NY).

Non-limiting examples of anti-GD3 antibodies include: Cat. No. ab11779 (Abcam, Waltham, MA); Cat. No. 14-9754-82 (ThermoFisher Scientific, Waltham, MA); Cat. No. 917701 (BioLegend, San Diego, CA); Cat. No. MABC1112 (Millipore Sigma, Burlington, MA); Cat. No. sc-33685 (Santa Cruz Biotechnology, Dallas, TX); antibodies provided by Hedberg et al. (2000) Glycoconjugate Journal 17:717-726.

In some embodiments, competitive ELISA is used to detect and measure the ganglioside levels. A competitive ELISA (also known as inhibition ELISA or competitive immunoassay) measures the concentration of an antigen by detection of signal interference. The sample antigen competes with a reference antigen for binding to a specific amount of labeled antibody. The reference antigen can be any one or more antigens of the present disclosure. Alternatively, the reference antigen can be any ganglioside or modified version thereof known in the art.

In some embodiments, the reference antigen is pre-coated on a multi-well plate. The sample is pre-incubated with labeled antibody (e.g., anti-GD2 or anti-GD3 antibody, e.g., an antibody of the present disclosure) and added to the wells. Depending on the amount of antigen in the sample, more or less free antibodies will be available to bind the reference antigen. This means the more antigen there is in the sample, the less reference antigen will be detected and the weaker the signal. Some competitive ELISA methods use labeled antigen instead of a labeled antibody. The labeled antigen and the sample antigen (unlabeled) compete for binding to the primary antibody. The lower the amount of antigen in the sample, the stronger the signal due to more labeled antigen in the well. Other conventional variations may also be employed as suitable.

In other embodiments, the competitive ELISA assay comprises coating a primary antibody (e.g., anti-GD2 or anti-GD3 antibody, e.g., an antibody of the present disclosure) on a multi-well plate. Here, a detectable/labeled reference antigen (e.g., FITC-labeled ganglioside) and the sample are co-incubated in the wells, and the labeled reference antigen competes with a native antigen for binding to the primary antibody. The lower the amount of antigen in the sample, the stronger the signal due to more labeled antigen in the well.

In yet other embodiments, the competitive ELISA assay comprises anchoring a primary antibody (e.g., anti-GD2 or anti-GD3 antibody, e.g., an antibody of the present disclosure) to the multi-well plate via an anti-species secondary antibody that is coated in the multi-well plate. For example, to anchor a primary antibody generated in mice, an anti-mouse secondary antibody is used. Then, a detectable/labeled reference antigen (e.g., FITC-labeled ganglioside) and the sample are co-incubated in the wells, and the labeled reference antigen competes with a native antigen for binding to the primary antibody. The lower the amount of antigen in the sample, the stronger the signal due to more labeled antigen in the well.

In some embodiments, a method for measuring biomarker ganglioside levels comprises the steps of: contacting a biological specimen (e.g., liquid biopsy (e.g., blood, serum)) with an antibody or variant (e.g., fragment) thereof which selectively binds the biomarker ganglioside, and detecting whether said antibody or variant thereof is bound to said sample and thereby measuring the levels of the biomarker ganglioside.

Immunohistochemistry may be used to detect the presence or the level of biomarker ganglioside, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabeling. The assay is scored visually, using microscopy.

Anti-ganglioside antibodies may also be used for imaging purposes, for example, to detect the presence of biomarker ganglioside on cells and tissues of a subject. Suitable labels include radioisotopes, iodine (¹²⁵I, ¹²¹I), carbon (¹⁴C), sulphur (³⁵S), tritium (³H), indium (¹¹²In), and technetium (⁹⁹mTc), fluorescent labels, such as fluorescein and rhodamine, and biotin.

Antibodies and derivatives thereof that may be used encompass polyclonal or monoclonal antibodies, chimeric, human, humanized, primatized (CDR-grafted), veneered or single-chain antibodies as well as functional fragments, i.e., biomarker protein binding fragments, of antibodies. For example, antibody fragments capable of binding to a biomarker protein or portions thereof, including, but not limited to, Fv, Fab, Fab′ and F(ab′) 2 fragments can be used.

In some embodiments, a mass spectrometry (e.g., MALDI-TOF, LC/MS/MS, LC/MS, or others known in the art) is used to detect the presence, level, or the lipid length of a ganglioside (e.g., GD1, GD2, GD3, and/or GM2) in a biological specimen, such as liquid biopsy (e.g., blood, saliva, serum, see the section on Samples). The mass spectrometry-based method is particularly useful in determining the heterogeneity of the lipid length of a ganglioside, which is a novel biomarker of the present disclosure.

The “level” or “amount” of a biomarker (e.g., a ganglioside) in a subject is “significantly” higher or lower than the level of a biomarker in a control, if the amount of the biomarker is greater or less, respectively, than the level in a control by an amount greater than the standard error of the assay employed to assess amount.

In some embodiments, the amount or level of a biomarker in a subject can be considered “significantly” higher or lower than the normal and/or control amount if the amount is at least or about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, 1000%, 1500%, 2000%, 2500%, 3000%, or more, or any range in between, such as 5%-100%, higher or lower, respectively, than the normal and/or control amount of the biomarker. Such significant modulation values can be applied to any metric described herein, such as the level of a ganglioside or a change in the heterogeneity of the lipid length of a ganglioside, and the like.

The term “heterogeneity of lipid length” of a at least one ganglioside includes the level or distribution pattern of the lipid length of at least one ganglioside in a given sample.

In some embodiments, there is a change (e.g., increase or decrease) or a significant change in the heterogeneity of lipid length, if the level or distribution pattern of the lipid length of at least one ganglioside in a subjection sample is at least or about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, 1000%, 1500%, 2000%, 2500%, 3000%, or more, or any range in between, such as 5%-100%, higher or lower than that in a normal and/or a control sample.

Similarly, the term “heterogeneity of a lipid length” of a ganglioside includes the distribution patterns of gangliosides having a short lipid length vs. a long lipid length (see Example 6). Gangliosides have two lipid tails: sphingosine and acyl. As used herein, two lipid tails are not distinguished. Accordingly, the term “heterogeneity of a lipid length” as used herein refers to the average length of both lipid tails of a ganglioside. In some embodiments, there is a change or a significant change in the heterogeneity of a lipid length of a ganglioside, if the amount or level of a ganglioside having a short lipid length (14-34 carbons) in a subject sample is at least or about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, 1000%, 1500%, 2000%, 2500%, 3000%, or more, or any range in between, such as 5%-100%, higher or lower than that in a normal and/or a control sample.

In some embodiments, there is a change or a significant change in the heterogeneity of a lipid length of a ganglioside, if the amount or level of a ganglioside having a long lipid length (36-48 carbons) in a subject sample is at least or about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, 1000%, 1500%, 2000%, 2500%, 3000%, or more, or any range in between, such as 5%-100%, higher or lower than that in a normal and/or a control sample.

In some embodiments, there is a change or a significant change in the heterogeneity of a lipid length of a ganglioside, if the amount or level of a ganglioside having a short lipid length (14-34 carbons) and a long lipid length (36-48 carbons) in a subject sample is at least or about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 250%, 300%, 350%, 400%, 450%, 500%, 550%, 600%, 650%, 700%, 750%, 800%, 850%, 900%, 950%, 1000%, 1500%, 2000%, 2500%, 3000%, or more, or any range in between, such as 5%-100%, higher or lower than that in a normal and/or a control sample.

### Control

A control refers to any suitable reference standard, such as a normal patient, cultured primary cells/tissues isolated from a subject such as a normal subject, adjacent normal cells/tissues obtained from the same organ or body location of the patient, a tissue or cell sample isolated from a normal subject, or a primary cells/tissues obtained from a depository. In other embodiments, the control may comprise an expression level (e.g., the level of a ganglioside) and/or lipid tail length of a ganglioside of a subject, such as a normal or healthy subject. In some embodiments, a control refers to a sample lacking the test agent (e.g., an anti-ganglioside antibody).

A control also refers to any reference standard suitable to provide a comparison to the expression products in the test sample. In certain embodiments, the control comprises obtaining a control sample from which the level or the lipid length of a ganglioside is detected and compared to the same from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In some embodiments, the control may comprise a reference standard expression product (e.g., ganglioside) level from any suitable source, including but not limited to an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present invention.

In some embodiments, the amount of proteins or nucleic acids may be determined within a sample relative to, or as a ratio of, the amount of proteins or nucleic acids of another gene in the same sample. In some embodiments, the control comprises a ratio transformation of expression product levels, including but not limited to determining a ratio of product levels of two gangliosides or the ratio of short lipid length vs. long lipid length of a ganglioside in the test sample and comparing it to any suitable ratio of the same in a reference standard; determining product levels of the two or more genes in the test sample and determining a difference in product levels in any suitable control; and determining product levels of the two or more gangliosides in the test sample, normalizing their level to the level of housekeeping gene products in the test sample, and comparing to any suitable control. In preferred embodiments, the control comprises a control sample which is of the same lineage and/or type as the test sample. In other embodiments, the control may comprise product levels grouped as percentiles within or based on a set of patient samples, such as all patients with cancer. In some embodiments, a control product level is established wherein higher or lower levels of product relative to, for instance, a particular percentile, are used as the basis for predicting outcome. In other preferred embodiments, a control product level is established using product levels from cancer control patients with a known outcome, and the product levels from the test sample are compared to the control product level as the basis for predicting outcome. As demonstrated by the data provided herein, the methods of the present invention are not limited to use of a specific cut-point in comparing the level of product in the test sample to the control.

In some embodiments, a pre-determined marker amount and/or activity measurement(s) can be any suitable standard. For example, the pre-determined marker amount and/or activity measurement(s) can be obtained from the same or a different human for whom a patient selection is being assessed. In some embodiments, the pre-determined marker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

### Diagnostic Assays

The present invention provides, in part, methods, systems, and code for accurately classifying whether a biological sample comprises a ganglioside and/or whether the levels of a ganglioside are modulated (e.g., upregulated or downregulated), thereby indicative of the state of a disorder of interest, such as cancer. In some embodiments, the present invention is useful for classifying a sample (e.g., from a subject) as associated with or at risk for cancer or a subtype thereof, mediated by a ganglioside using a statistical algorithm and/or empirical data (e.g., the presence, absence, level, or the lipid length of a ganglioside).

An exemplary method for detecting the level of a ganglioside, and thus useful for classifying whether a sample is associated with a cancer or a clinical subtype thereof or different stages of a cancer involves obtaining a biological sample from a test subject and contacting the biological sample with an antibody or antigen-binding fragment thereof of the present invention capable of detecting a ganglioside such that the level of ganglioside is detected in the biological sample. In some embodiments, at least one antibody or antigen-binding fragment thereof is used, wherein two, three, four, five, six, seven, eight, nine, ten, or more such antibodies or antibody fragments can be used in combination (e.g., in sandwich ELISAs) or in serial. In certain instances, the statistical algorithm is a single learning statistical classifier system. For example, a single learning statistical classifier system can be used to classify a sample as a cancer sample based upon a prediction or probability value and the presence or level of ganglioside. The use of a single learning statistical classifier system typically classifies the sample as a cancer sample with a sensitivity, specificity, positive predictive value, negative predictive value, and/or overall accuracy of at least or about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naïve learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the sample classification results to a clinician (a non-specialist, e.g., primary care physician; and/or a specialist, e.g., a histopathologist or an oncologist).

In some embodiments, the method of the present disclosure further provides a diagnosis in the form of a probability that the individual has a cancer. For example, the individual can have about a 0%, 5%, 10%, 15%, 20%, 25%, 30%, 35%, 40%, 45%, 50%, 55%, 60%, 65%, 70%, 75%, 80%, 85%, 90%, 95%, or greater probability of having the cancer. In yet another embodiment, the method of the present invention further provides a prognosis of the cancer in the individual. In some instances, the method of classifying a sample as a cancer sample may be further based on the symptoms (e.g., clinical factors) of the individual from which the sample is obtained. The symptoms or group of symptoms can be, for example, lymphocyte count, white cell count, erythrocyte sedimentation rate, diarrhea, abdominal pain, bloating, pelvic pain, lower back pain, cramping, fever, anemia, weight loss, anxiety, depression, and combinations thereof. In some instances, the method of classifying a sample as a cancer sample may be further based on genetic mutations and/or predisposition to cancer, irrespective of the symptoms. In some embodiments, the diagnosis of an individual as having a cancer is followed by administering to the individual a therapeutically effective amount of a cancer therapy (e.g., chemotherapeutic agents).

An exemplary method for detecting the presence or absence of a ganglioside comprises using an antibody of the present disclosure, or fragment thereof, capable of binding to a ganglioside, preferably an antibody with a detectable label. Antibodies can be polyclonal, or more preferably, monoclonal. Such agents can be labeled. The term “labeled”, with regard to the antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody. The term “biological sample” is intended to include tissues, cells, and biological fluids isolated from a subject, such as serum, blood, as well as tissues, cells, and fluids present within a subject. That is, the detection method of the present disclosure can be used to detect a ganglioside in a biological sample in vitro, ex vivo, as well as in vivo. In vitro techniques for detection of a ganglioside include enzyme linked immunosorbent assays (ELISAs), immunoprecipitations, immunohistochemistry (IHC), flow cytometry and related techniques, and immunofluorescence. Furthermore, in vivo techniques for detection of a ganglioside include introducing into a subject a labeled anti-ganglioside antibody. For example, the antibody can be labeled with a radioactive, luminescent, fluorescent, or other similar marker whose presence and location in a subject can be detected by standard imaging techniques, either alone or in combination with imaging for other molecules, such as markers of cell type (e.g., CD8+ T cell markers).

Another exemplary method for detecting the presence, level, or the lipid length of a ganglioside is using a mass spectrometry, preferably coupled with HPLC.

In some embodiments, the methods further involve obtaining a control biological sample (e.g., biological sample from a subject who does not have a cancer), a biological sample from the subject during remission or before developing a cancer, or a biological sample from the subject during treatment for developing a cancer.

In some embodiments, the methods comprise contacting the control sample with a compound or agent capable of detecting a ganglioside such that the presence and/or the level of a ganglioside is detected in the biological sample, and comparing the presence or the level of a ganglioside in the control sample with the presence or the level of a ganglioside in the test sample.

A preferred biological sample is a serum, blood, saliva, tumor microenvironment, peritumoral, or intratumoral, isolated by conventional means from a subject.

In still other embodiments, the antibodies can be associated with a component or device for the use of the antibodies in an ELISA or RIA. Non-limiting examples include antibodies immobilized on solid surfaces for use in these assays (e.g., linked and/or conjugated to a detectable label based on light or radiation emission as described above). In other embodiments, the antibodies are associated with a device or strip for detection of a ganglioside by use of an immunochromatographic or immunochemical assay, such as in a “sandwich” or competitive assay, immunohistochemistry, immunofluorescence microscopy, and the like. Additional examples of such devices or strips are those designed for home testing or rapid point of care testing. Further examples include those that are designed for the simultaneous analysis of multiple analytes in a single sample. For example, an unlabeled antibody of the invention may be applied to a “capture” a ganglioside in a biological sample and the captured (or immobilized) ganglioside may be bound to a labeled form of an anti-ganglioside antibody of the present disclosure for detection. Other embodiments of immunoassays are well-known the skilled artisan, including assays based on, for example, immunodiffusion, immunoelectrophoresis, immunohistopathology, immunohistochemistry, and histopathology.

The level and/or heterogeneity of the lipid length of at least one ganglioside, as determined by the compositions and methods of the present disclosure, correlate with different grades of a cancer. Accordingly, in some embodiments, the compositions and methods of the present disclosure can be used to determine a grade of a cancer, based on the level and/or heterogeneity of the lipid length of at least one ganglioside determined as described herein. A cancer's grade describes how abnormal the cancer cells and tissue look under a microscope when compared to healthy cells. Cancer cells that look and organize most like healthy cells and tissue are low grade tumors. Doctors describe these cancers as being well differentiated. Lower grade cancers are typically less aggressive and have a better prognosis. The more abnormal the cells look and organize themselves, the higher the cancer's grade. Cancer cells with a high grades tend to be more aggressive. They are called poorly differentiated or undifferentiated. Some cancers have their own system for grading tumors. Many others use a standard 1-4 grading scale.
Grade 1: Tumor cells and tissue looks most like healthy cells and tissue. These are called well-differentiated tumors and are considered low grade.
Grade 2: The cells and tissue are somewhat abnormal and are called moderately differentiated. These are intermediate grade tumors.
Grade 3: Cancer cells and tissue look very abnormal. These cancers are considered poorly differentiated, since they no longer have an architectural structure or pattern. Grade 3 tumors are considered high grade.
Grade 4: These undifferentiated cancers have the most abnormal looking cells. These are the highest grade and typically grow and spread faster than lower grade tumors.

As used herein, low grade cancer refers to Grade I cancer; and high grade cancer refers to cancer of Grades 2-4.

Similarly, the level and/or heterogeneity of the lipid length of at least one ganglioside, as determined by the compositions and methods of the present disclosure, correlate with different stages of a cancer. Accordingly, in some embodiments, the compositions and methods of the present disclosure can be used to determine a grade of a cancer, based on the level and/or heterogeneity of the lipid length of at least one ganglioside determined as described herein.

A cancer's stage explains how large the primary tumor is and how far the cancer has spread in the patient's body. There are several different staging systems. Many of these have been created for specific kinds of cancers. Others can be used to describe several types of cancer. One common system that many people are aware of puts cancer on a scale of 0 to IV.
Stage 0 is for abnormal cells that haven't spread and are not considered cancer, though they could become cancerous in the future. This stage is also called “in-situ.”
Stage I through Stage III are for cancers that haven't spread beyond the primary tumor site or have only spread to nearby tissue. The higher the stage number, the larger the tumor and the more it has spread.
Stage IV cancer has spread to distant areas of the body.

As used herein, cancer at the early/low stage refers to cancer at Stage I; and cancer at the late/high/advanced stage includes cancer at Stage II to Stage IV.

Likewise, the level and/or heterogeneity of the lipid length of at least one ganglioside, as determined by the compositions and methods of the present disclosure, correlate with the tumor burden. Accordingly, in some embodiments, the compositions and methods of the present disclosure can be used to determine the tumor burden of a subject, based on the level and/or heterogeneity of the lipid length of at least one ganglioside determined as described herein. Tumor burden (or tumor load) is defned as the total amount of tumor (cells/mass) distributed in the patients' body, including bone marrow. In Response Evaluation Criteria in Solid Tumors (RECIST) analysis, tumor burden is considered the sum of the longest diameters of all measurable lesions. Various methods can be used to determine the tumor burden in a subject. For example, computed tomography (CT) and magnetic resonance (MR) imaging have been used to assess tumor response based on morphologic (size, location) criteria, specifcally by using RECIST. RECIST classification describes lesions' size and distinguishes 4 types of treatment response-stable disease (SD), partial response (PR), complete response (CR) or progressive disease (PD).

### Prognostic Assays

The term “prognosis” includes a prediction of the probable course and outcome of cancer or the likelihood of recovery from the disease. In some embodiments, the use of statistical algorithms provides a prognosis of cancer in an individual. For example, the prognosis can be surgery, development of a clinical subtype of cancer (e.g., solid tumors, such as lung cancer, melanoma, and renal cell carcinoma), development of one or more clinical factors, development of intestinal cancer, or recovery from the disease.

The assays described herein, such as the preceding diagnostic assays or the following assays, can be utilized to to determine whether a subject can be administered an agent (e.g., an agonist, antagonist, peptidomimetic, polypeptide, peptide, nucleic acid, small molecule, immunotherapy, immune checkpoint inhibition therapy, or other drug candidate) to treat a cancer. For example, such methods can be used to determine whether a subject can be effectively treated with one or a combination of agents. Thus, the present disclosure provides methods for determining whether a subject can be effectively treated with one or more agents for treating a cancer in which a test sample is obtained and a ganglioside is detected.

The methods described herein may be performed, for example, by utilizing pre-packaged diagnostic kits comprising at least one antibody reagent described herein, which may be conveniently used, e.g., in clinical settings to diagnose patients exhibiting symptoms or family history of a cancer.

Other aspects of the present disclosure include uses of the compositions and methods described herein for association and/or stratification analyses in which a ganglioside in biological samples from individuals with a cancer, are analyzed and the information is compared to that of controls (e.g., individuals who do not have the cancer; controls may be also referred to as “healthy” or “normal” individuals or at early timepoints in a given time lapse study) who are preferably of similar age and race. The appropriate selection of patients and controls is important to the success of association and/or stratification studies. Therefore, a pool of individuals with well-characterized phenotypes is extremely desirable. Criteria for cancer diagnosis, cancer predisposition screening, predicting clinical outcomes, cancer prognosis, determining drug responsiveness (pharmacogenomics), drug toxicity screening, etc. are described herein.

Different study designs may be used for genetic association and/or stratification studies (Modern Epidemiology, Lippincott Williams & Wilkins (1998), 609-622). Observational studies are most frequently carried out in which the response of the patients is not interfered with. The first type of observational study identifies a sample of persons in whom the suspected cause of the disease is present and another sample of persons in whom the suspected cause is absent, and then the frequency of development of disease in the two samples is compared. These sampled populations are called cohorts, and the study is a prospective study. The other type of observational study is case-control or a retrospective study. In typical case-control studies, samples are collected from individuals with the phenotype of interest (cases) such as certain manifestations of a disease, and from individuals without the phenotype (controls) in a population (target population) that conclusions are to be drawn from. Then the possible causes of the disease are investigated retrospectively. As the time and costs of collecting samples in case-control studies are considerably less than those for prospective studies, case-control studies are the more commonly used study design in genetic association studies, at least during the exploration and discovery stage.

After all relevant phenotypic and/or genotypic information has been obtained, statistical analyses are carried out to determine if there is any significant correlation between the presence of an allele or a genotype with the phenotypic characteristics of an individual. Preferably, data inspection and cleaning are first performed before carrying out statistical tests for genetic association. Epidemiological and clinical data of the samples can be summarized by descriptive statistics with tables and graphs well-known in the art. Data validation is preferably performed to check for data completion, inconsistent entries, and outliers. Chi-squared tests and t-tests (Wilcoxon rank-sum tests if distributions are not normal) may then be used to check for significant differences between cases and controls for discrete and continuous variables, respectively.

An important decision in the performance of genetic association tests is the determination of the significance level at which significant association can be declared when the p-value of the tests reaches that level. In an exploratory analysis where positive hits will be followed up in subsequent confirmatory testing, an unadjusted p-value <0.2 (a significance level on the lenient side), for example, may be used for generating hypotheses for significant association of a ganglioside level with certain phenotypic characteristics of a cancer. It is preferred that a p-value <0.05 (a significance level traditionally used in the art) is achieved in order for the level to be considered to have an association with a cancer. When hits are followed up in confirmatory analyses in more samples of the same source or in different samples from different sources, adjustment for multiple testing will be performed as to avoid excess number of hits while maintaining the experiment-wise error rates at 0.05. While there are different methods to adjust for multiple testing to control for different kinds of error rates, a commonly used but rather conservative method is Bonferroni correction to control the experiment-wise or family-wise error rate (Multiple comparisons and multiple tests, Westfall et al, SAS Institute (1999)). Permutation tests to control for the false discovery rates, FDR, can be more powerful (Benjamini and Hochberg, Journal of the Royal Statistical Society, Series B 57, 1289-1300, 1995, Resampling-based Multiple Testing, Westfall and Young, Wiley (1993)). Such methods to control for multiplicity would be preferred when the tests are dependent and controlling for false discovery rates is sufficient as opposed to controlling for the experiment-wise error rates.

Once individual risk factors, genetic or non-genetic, have been found for the predisposition to disease, a classification/prediction scheme can be set up to predict the category (for instance, disease or no-disease) that an individual will be in depending on his phenotype and/or genotype and other non-genetic risk factors. Logistic regression for discrete trait and linear regression for continuous trait are standard techniques for such tasks (Applied Regression Analysis, Draper and Smith, Wiley (1998)). Moreover, other techniques can also be used for setting up classification. Such techniques include, but are not limited to, MART, CART, neural network, and discriminant analyses that are suitable for use in comparing the performance of different methods (The Elements of Statistical Learning, Hastie, Tibshirani & Friedman, Springer (2002)).

### Exemplary Embodiments of the Diagnostic and Prognostic Methods

In certain aspects, provided herein are diagnostic and prognostic methods. For example, in certain aspects, provided herein is a method of diagnosing a cancer in a subject, the method comprising: a) determining the level of at least one ganglioside in a subject sample; and b) comparing said level of the at least one ganglioside to that in a control sample, wherein a significantly higher level of the at least one ganglioside in the subject sample as compared to the level in the control sample indicates that the subject has a cancer.

In certain aspects, provided herein is a method of identifying a subject having a cancer, the method comprising: a) determining the level of at least one ganglioside in a subject sample; and b) comparing said level of the at least one ganglioside to that in a control sample, wherein a significantly higher level of the at least one ganglioside in the subject sample as compared to the level in the control sample identifies the subject as having a cancer.

In certain aspects, provided herein is a method of determining a stage of a cancer, the method comprising: a) determining the level of at least one ganglioside in a subject sample; and b) comparing said level of the at least one ganglioside to that in a control sample, wherein at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% increase in the level of the at least one ganglioside in the subject sample as compared to the level in the control sample indicates that the subject has an early-stage cancer; and/or wherein at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% increase in the level of the at least one ganglioside in the subject sample as compared to the level in the control sample indicates that the subject has a late-stage cancer.

In some embodiments, at least 100% and no more than 200% increase in the level of the at least one ganglioside in the subject sample as compared to the level in the control sample indicates that the subject has an early-stage cancer. In some embodiments, at least 200% increase in the level of the at least one ganglioside in the subject sample as compared to the level in the control sample indicates that the subject has a late-stage cancer.

In certain aspects, provided herein is a method of determining a grade of a cancer, the method comprising: a) determining the level of at least one ganglioside in a subject sample; and b) comparing said level of the at least one ganglioside to that in a control sample, wherein at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% increase in the level of the at least one ganglioside in the subject sample as compared to the level in the control sample indicates that the subject has a Grade I cancer; wherein at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% increase in the level of the at least one ganglioside in the subject sample as compared to the level in the control sample indicates that the subject has a Grade II cancer; wherein at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% increase in the level of the at least one ganglioside in the subject sample as compared to the level in the control sample indicates that the subject has a Grade III cancer; and/or wherein at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% increase in the level of the at least one ganglioside in the subject sample as compared to the level in the control sample indicates that the subject has a Grade IV cancer.

In some embodiments, at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% increase in the level of the at least one ganglioside in the subject sample as compared to the level in the control sample indicates that the subject has a low grade cancer (e.g., Grade I).

In some embodiments, at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% increase in the level of the at least one ganglioside in the subject sample as compared to the level in the control sample indicates that the subject has an high grade cancer (e.g., Grade II, III, or IV).

In some embodiments, at least 100% and no more than 200% increase in the level of the at least one ganglioside in the subject sample as compared to the level in the control sample indicates that the subject has a low grade cancer (e.g., Grade I). In some embodiments, at least 200% increase in the level of the at least one ganglioside in the subject sample as compared to the level in the control sample indicates that the subject has a high-grade cancer (e.g., Grade II, III, or IV).

In certain aspects, provided herein is a method of determining a tumor burden of a cancer, the method comprising: a) determining the level of at least one ganglioside in a subject sample; and b) comparing said level of the at least one ganglioside to that in a control sample, wherein at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% increase in the level of the at least one ganglioside in the subject sample as compared to the level in the control sample indicates that the subject has a low tumor burden; and/or wherein at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% increase in the level of the at least one ganglioside in the subject sample as compared to the level in the control sample indicates that the subject has a high tumor burden.

In some embodiments, at least 100% and no more than 200% increase in the level of the at least one ganglioside in the subject sample as compared to the level in the control sample indicates that the subject has a low tumor burden. In some embodiments, at least 200% increase in the level of the at least one ganglioside in the subject sample as compared to the level in the control sample indicates that the subject has a high tumor burden.

In certain aspects, provided herein is a method of detecting a recurrence of a cancer in a subject, the method comprising: a) obtaining or providing a sample from the subject whose cancer has regressed after receiving cancer treatment; b) determining the level of at least one ganglioside in the subject sample; and c) comparing said level of the at least one ganglioside to that in a control sample, wherein a significantly higher level of the at least one ganglioside in the subject sample as compared to the level in the control sample indicates a recurrence of a cancer in the subject.

In certain aspects, provided herein is a method of detecting a minimal residual disease in a subject, the method comprising: a) obtaining or providing a sample from the subject in remission; b) determining the level of at least one ganglioside in the subject sample; and c) comparing said level of the at least one ganglioside to that in a control sample, wherein a significantly higher level of the at least one ganglioside in the subject sample as compared to the level in the control sample indicates that the subject has a minimal residual disease.

In certain aspects, provided herein is a method of stratifying subjects afflicted with a cancer according to benefit from a cancer therapy (e.g., immunotherapy), the method comprising: a) determining the level of at least one ganglioside in a subject sample; b) determining the level of the at least one ganglioside in a control; and c) comparing the level of the at least one ganglioside detected in steps a) and b); wherein no significant change or a decrease in the level of the at least one ganglioside in the subject sample as compared to the level in the control is an indication that the subject afflicted with the cancer would benefit from the cancer therapy.

In certain aspects, provided herein is a method of determining whether a subject afflicted with a cancer would likely respond or alternatively would likely not respond to a cancer therapy (e.g., immunotherapy), the method comprising: a) determining the level of at least one ganglioside in a subject sample; b) determining the level of the at least one ganglioside in a control; and c) comparing the level of the at least one ganglioside detected in steps a) and b); wherein a significantly higher level of the at least one ganglioside in the subject sample as compared to the level in the control is an indication that the subject afflicted with the cancer would not respond to the cancer therapy; and/or wherein no significant change or a decrease in the level of the at least one ganglioside in the subject sample as compared to the level in the control is an indication that the subject afflicted with the cancer would respond to the cancer therapy.

In certain aspects, provided herein is a method for predicting the clinical outcome of a subject afflicted with a cancer, the method comprising: a) determining the level of at least one ganglioside in a subject sample; b) determining the level of the at least one ganglioside in a control; and c) comparing the level of the at least one ganglioside determined in steps a) and b); wherein a significantly higher level of the at least one ganglioside in the subject sample as compared to the level in the control is an indication that the subject has a poor clinical outcome.

In certain aspects, provided herein is a method of monitoring the progression of a cancer in a subject, the method comprising: a) detecting in a subject sample at a first point in time the level of at least one ganglioside; b) repeating step a) at a subsequent point in time; and c) comparing the level of the at least one ganglioside detected in steps a) and b) to monitor the progression of the cancer in the subject.

In some embodiments, the method monitors the progression of a cancer in a subject who has received a cancer therapy between the first point in time and the subsequent point in time. In some embodiments, the subject is at risk for developing a cancer.

In certain aspects, provided herein is a method of assessing the efficacy of a cancer therapy in a subject, the method comprising: a) determining the level of at least one ganglioside in a first sample obtained from a subject; b) repeating step a) during at least one subsequent point in time after administration of the cancer therapy; and c) comparing the level of at least one ganglioside detected in steps a) and b), wherein a significantly lower level of the at least one ganglioside in the at least one subsequent sample, relative to the first sample, is an indication that the therapy is efficacious to treat a cancer in the subject.

In some embodiments, the first and/or at least one subsequent sample is a portion of a single sample or pooled samples obtained from the subject.

In some embodiments, the cancer therapy is a surgery, chemotherapy, cancer vaccines, chimeric antigen receptors, radiation therapy, immunotherapy, a modulator of expression of immune checkpoint inhibitory proteins or ligands, or any combination thereof. In some embodiments, the immunotherapy is an immune checkpoint inhibition therapy. In some embodiments, the cancer therapy is avelumab, durvalumab, atezolizumab, BRAF/MEK inhibitor, a tyrosine kinase inhibitor, pembrolizumab, nivolumab, ipilimumab, or a combination thereof.

Numerous embodiments are further provided that can be applied to any aspect of the present invention described herein.

For example, the diagnostic and prognostic methods described herein may use any method known in the art to detect and determine the level of at least one ganglioside. In some embodiments, the at least one ganglioside is detected and its level is determined by at least one monoclonal antibody, or antigen-binding fragment thereof. In preferred embodiments, the at least one ganglioside is detected and its level is determined by at least one monoclonal antibody, or antigen-binding fragment thereof, described herein, using any one of exemplary methods described herein or those known in the art (e.g., ELISA, sandwich ELISA, competitive ELISA).

In yet other preferred embodiments, the at least one ganglioside is detected and its level is determined by mass spectrometry (e.g., LC/MS, LC/MS/MS, or any other mass spectrometry method known in the art).

In some embodiments, at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% higher level of the at least one ganglioside indicates a significantly higher level.

In some embodiments, at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% lower level of the at least one ganglioside indicates a significantly lower level.

In some embodiments, at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% increase or decrease in the level of at least one ganglioside indicates no significant change in the level of the at least one ganglioside.

Further provided herein are diagnostic and prognostic methods that use the heterogeneity or homogeneity of the lipid length (e.g., as determined using mass spectrometry) of at least one ganglioside. In some embodiments, a significant change in heterogeneity of the lipid length of the at least one ganglioside in the subject sample as compared to the control sample indicates that the subject has a cancer. Similarly, in some embodiments, a significant change in the heterogeneity of the lipid length of the at least one ganglioside in the subject sample as compared to the control sample identifies the subject as having a cancer.

In certain aspects, provided herein is a method of determining a stage of a cancer, the method comprising: a) determining the lipid length of at least one ganglioside in the subject sample using mass spectrometry; and b) comparing said lipid length of the at least one ganglioside to that in a control sample, wherein at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% change in the heterogeneity of the lipid length of the at least one ganglioside in the subject sample as compared to that in the control sample indicates that the subject has an early-stage cancer; and/or at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% change in the heterogeneity of the lipid length of the at least one ganglioside in the subject sample as compared to that in the control sample indicates that the subject has a late-stage cancer.

In some embodiments, at least 100% and no more than 200% change (e.g., increase or decrease) in heterogeneity of the lipid length of the at least one ganglioside in the subject sample as compared to that in the control sample indicates that the subject has an early-stage cancer. In some embodiments, at least 200% change (e.g., increase or decrease) in heterogeneity of the lipid length of the at least one ganglioside in the subject sample as compared to that in the control sample indicates that the subject has a late-stage cancer.

In some embodiments, at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% increase or decrease in (a) the level of a ganglioside having a short lipid length with respect to the level of a ganglioside having a long lipid length; or (b) the level of a ganglioside having a long lipid length with respect to the level of a ganglioside having a short lipid length; indicates that the subject has an early-stage or a late-stage cancer.

In certain aspects, provided herein is a method of determining a grade of a cancer, the method comprising: a) determining the lipid length of at least one ganglioside in the subject sample using mass spectrometry; and b) comparing the said lipid length of the at least one ganglioside to that in a control sample, wherein at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% change (e.g., increase or decrease) in heterogeneity of the lipid length of the at least one ganglioside in the subject sample as compared to that in the control sample indicates that the subject has a Grade I, Grade II, Grade III, or Grade IV cancer.

In some embodiments, at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% change (e.g., increase or decrease) in heterogeneity of the lipid length of the at least one ganglioside in the subject sample as compared to that in the control sample indicates that the subject has a low grade cancer or a high grade cancer.

In some embodiments, at least 100% and no more than 200% change (e.g., increase or decrease) in heterogeneity of the lipid length of the at least one ganglioside in the subject sample as compared to that in the control sample indicates that the subject has a low grade cancer. In some embodiments, at least 200% change (e.g., increase or decrease) in heterogeneity of the lipid length of the at least one ganglioside in the subject sample as compared to that in the control sample indicates that the subject has a high grade cancer.

In some embodiments, at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% increase or decrease in (a) the level of a ganglioside having a short lipid length with respect to the level of a ganglioside having a long lipid length; or (b) the level of a ganglioside having a long lipid length with respect to the level of a ganglioside having a short lipid length; indicates that the subject has a low grade or a high grade cancer.

In certain aspects, provided herein is a method of determining a tumor burden of a cancer, the method comprising: a) determining the lipid length of at least one ganglioside in the subject sample using mass spectrometry; and b) comparing said lipid length of the at least one ganglioside to that in a control sample, wherein at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% change in the heterogeneity of the lipid length of the at least one ganglioside in the subject sample as compared to that in the control sample indicates that the subject has low tumor burden; and/or at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% change in the heterogeneity of the lipid length of the at least one ganglioside in the subject sample as compared to that in the control sample indicates that the subject has a high tumor burden.

In some embodiments, at least 100% and no more than 200% change (e.g., increase or decrease) in heterogeneity of the lipid length of the at least one ganglioside in the subject sample as compared to that in the control sample indicates that the subject has a low tumor burden. In some embodiments, at least 200% change (e.g., increase or decrease) in heterogeneity of the lipid length of the at least one ganglioside in the subject sample as compared to that in the control sample indicates that the subject has a high tumor burden.

In some embodiments, at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% increase or decrease in (a) the level of a ganglioside having a short lipid length with respect to the level of a ganglioside having a long lipid length; or (b) the level of a ganglioside having a long lipid length with respect to the level of a ganglioside having a short lipid length; indicates that the subject has low tumor burden or a high tumor burden.

In certain aspects, provided is a method of detecting a recurrence of a cancer in a subject, the method comprising: a) obtaining or providing a sample from the subject whose cancer has regressed after receiving cancer treatment; b) determining the lipid length of at least one ganglioside in the subject sample using mass spectrometry; and c) comparing the said lipid length of the at least one ganglioside to that in a control sample, wherein a significant change in heterogeneity of the lipid length of the at least one ganglioside in the subject sample as compared to that in the control sample indicates a recurrence of a cancer in the subject.

In certain aspects, provided herein is a method of detecting a minimal residual disease in a subject, the method comprising: a) obtaining or providing a sample from the subject in remission; b) determining the lipid length of at least one ganglioside in the subject sample using mass spectrometry; and c) comparing the said lipid length of the at least one ganglioside to that in a control sample, wherein a significant change in heterogeneity of the lipid length of the at least one ganglioside in the subject sample as compared to that in the control sample indicates that the subject has a minimal residual disease.

In certain aspects, provided herein is a method of stratifying subjects afflicted with a cancer according to benefit from a cancer therapy (e.g., immunotherapy), the method comprising: a) determining the lipid length of at least one ganglioside in a subject sample using mass spectrometry; b) determining the lipid length of the at least one ganglioside in a control; and c) comparing the the lipid length of the at least one ganglioside detected in steps a) and b); wherein no significant change in heterogeneity of the lipid length of the at least one ganglioside in the subject sample as compared to that in the control is an indication that the subject afflicted with the cancer would benefit from the cancer therapy.

In certain aspects, provided herein is a method of determining whether a subject afflicted with a cancer would likely respond to a cancer therapy, the method comprising: a) determining the lipid length of at least one ganglioside in a subject sample using mass spectrometry; b) determining the lipid length of the at least one ganglioside in a control; and c) comparing the lipid length of the at least one ganglioside detected in steps a) and b); wherein a significant change in heterogeneity of the lipid length of the at least one ganglioside in the subject sample as compared to that in the control is an indication that the subject afflicted with the cancer would not respond to the cancer therapy; and/or wherein no significant change in heterogeneity of the lipid length of the at least one ganglioside in the subject sample as compared to that in the control is an indication that the subject afflicted with the cancer would respond to the cancer therapy.

In certain aspects, provided herein is a method for predicting the clinical outcome of a subject afflicted with a cancer, the method comprising: a) determining the lipid length of at least one ganglioside in a subject sample using mass spectrometry; b) determining the lipid length of the at least one ganglioside in a control; and c) comparing the lipid length of the at least one ganglioside determined in steps a) and b); wherein a significant change in heterogeneity of the lipid length of the at least one ganglioside in the subject sample as compared to the control sample is an indication that the subject has a poor clinical outcome.

In certain aspects, provided herein is a method of monitoring the progression of a cancer in a subject, the method comprising: a) detecting in a subject sample at a first point in time the lipid length of at least one ganglioside using mass spectrometry; b) repeating step a) at a subsequent point in time; and c) comparing heterogeneity of the lipid length of the at least one ganglioside detected in steps a) and b) to monitor the progression of the cancer in the subject, optionally wherein the subject is at risk for developing a cancer.

In some embodiments, between the first point in time and the subsequent point in time, the subject has received a cancer therapy.

In certain aspects, provided herein is a method of assessing the efficacy of a cancer therapy in a subject, the method comprising: a) determining the lipid length of at least one ganglioside using mass spectrometry in a first sample obtained from a subject; b) repeating step a) during at least one subsequent point in time after administration of the cancer therapy; and c) comparing the level of at least one ganglioside detected in steps a) and b), wherein a significant change in heterogeneity of the lipid length of the at least one ganglioside in the second sample, relative to the first sample, is an indication that the therapy is efficacious to treat a cancer in the subject.

In some embodiments, the first and/or at least one subsequent sample is a portion of a single sample or pooled samples obtained from the subject.

The diagnostic and prognostic methods described herein may use any method known in the art to detect and determine the heterogeneity of the lipid length of the at least one ganglioside. In preferred embodiments, the heterogeneity of the lipid length of the at least one ganglioside is determined by mass spectrometry (e.g., LC/MS, LC/MS/MS, or any other mass spectrometry method known in the art).

In some embodiments, at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% change in the heterogeneity or homogeneity of the lipid length of the at least one ganglioside indicates a significant change in heterogeneity or homogeneity.

In some embodiments, at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% increase or decrease in (a) the level of a ganglioside having a short lipid length with respect to the level of a ganglioside having a long lipid length; or (b) the level of a ganglioside having a long lipid length with respect to the level of a ganglioside having a short lipid length; indicates a significant change in the heterogeneity or homogeneity of the lipid length.

In some embodiments, at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% change in the heterogeneity or homogeneity of the lipid length of the at least one ganglioside indicates no significant change in heterogeneity or homogeneity.

In some embodiments, at least, about, or no more than 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000% increase or decrease in (a) the level of a ganglioside having a short lipid length with respect to the level of a ganglioside having a long lipid length; or (b) the level of a ganglioside having a long lipid length with respect to the level of a ganglioside having a short lipid length; indicates no significant change in the heterogeneity or homogeneity of the lipid length.

Numerous embodiments are further provided that can be applied to any aspect of the present invention described herein. For example, in some embodiments, the cancer therapy is a surgery, chemotherapy, cancer vaccines, chimeric antigen receptors, radiation therapy, immunotherapy, a modulator of expression of immune checkpoint inhibitory proteins or ligands, or any combination thereof. In some embodiments, the immunotherapy is an immune checkpoint inhibition therapy. In some embodiments, the cancer therapy is avelumab, durvalumab, atezolizumab, BRAF/MEK inhibitor, a tyrosine kinase inhibitor, pembrolizumab, nivolumab, ipilimumab, or a combination thereof.

In some embodiments, a ganglioside is a tumor-associated ganglioside. In some embodiments, the tumor-associated ganglioside is selected from GD2, GD3, GD1b, GT1b, fucosyl-GM1, GloboH, polysialic acid (PSA), GM2, GM3, sialyl-Lewis^{X}, sialyl-Lewis^{Y}, sialyl-Lewis^{A}, sialyl-Lewis^{B}, Lewis^{Y}, any portion thereof; optionally wherein the tumor-associated ganglioside is selected from GD1, GD2, GD3, GT1b, and GM2.

In some embodiments, the cancer is selected from the group consisting of neuroblastoma, lymphoma, leukemia, melanoma, glioma, small cell lung cancer, breast carcinoma, ovarian cancer, soft tissue sarcomas, osteosarcoma, Ewing's sarcoma, desmoplastic round cell tumor, rhabdomyosarcoma, retinoblastoma, non-small cell lung cancer, renal cell cancer, Wilms tumor, prostate cancer, gastric cancer, endometrial cancer, pancreatic cancer, and colon cancer.

In some embodiments, the cancer is selected from the group consisting of neuroblastoma, lymphoma, leukemia, melanoma, glioma, small cell lung cancer, breast carcinoma, ovarian cancer, soft tissue sarcomas, osteosarcoma, Ewing's sarcoma, desmoplastic round cell tumor, rhabdomyosarcoma, retinoblastoma.

In some embodiments, the sample comprises cells, serum, blood, peritumoral tissue, and/or intratumoral tissue obtained from the subject (e.g., biopsy). In preferred embodiments, the sample comprises liquid biopsy (comprising liquid).

In some embodiments, a significantly higher level of at least one ganglioside comprises an at least twenty percent increase of the level of the at least one ganglioside.

In some embodiments, a significantly lower level of at least one ganglioside comprises an at least twenty percent decrease of the level of the at least one ganglioside. In preferred embodiments, a significantly higher or lower level of at least one ganglioside is the level that is at least or about 50% higher or lower than the level of control (e.g., a non-cancerous sample). In other embodiments, a significantly higher or lower level of at least one ganglioside is the level that is at least or about 25% higher or lower than the level of the previous reading of the subject in a longitudinal study. In some embodiments, a significant change in the heterogeneity of lipid length of at least one ganglioside comprises an at least twenty percent change (e.g., increase or decrease) in the subject sample relative to the control sample.

In some embodiments, the control sample is a sample from a cancer-free subject.

In some embodiments, the diagnostic and/or prognostic methods described herein further comprise recommending, prescribing, and/or administering to the subject a cancer therapy (e.g., immune checkpoint inhibition therapy).

In some embodiments, the subject is a mammal. The carbohydrate moiety of a ganglioside (e.g., GD2) is highly conserved (i.e., identical) in all mammals, thus, can be used to diagnose a cancer in all mammals (e.g., humans, pets, livestock).

In some embodiments, the subject is an animal model of cancer or a human.

In preferred embodiments, the subject is a human.

### Monitoring of Effects During Clinical Trials

Monitoring the influence of agents (e.g., compounds, drugs or small molecules) on the level of a ganglioside can be applied not only in basic drug screening, but also in clinical trials. For example, the effectiveness of an agent determined by a screening assay as described herein to decrease the level of a ganglioside can be monitored in clinical trials of subjects, detectable by the anti-ganglioside antibodies or fragments described herein or by the mass spectrometry-based methods. In such clinical trials, the level of a ganglioside and/or symptoms or other markers of the cancer, can be used as a “read out” or marker of the phenotype of a particular cell, tissue, or system.

In preferred embodiments, the present disclosure provides a method for monitoring the effectiveness of treatment of a subject with an agent (e.g., an agonist, antagonist, peptidomimetic, polypeptide, peptide, nucleic acid, small molecule, immunotherapy, immune checkpoint inhibition therapy, or other drug candidate) including the steps of (i) obtaining a pre-administration sample from a subject prior to administration of the agent; (ii) detecting the level of at least one ganglioside in the preadministration sample; (iii) obtaining one or more post-administration samples from the subject; (iv) detecting the level of the at least one ganglioside in the post-administration samples; (v) comparing the level of the at least one ganglioside in the pre-administration sample with the at least one ganglioside in the post administration sample or samples; and (vi) altering the administration of the agent to the subject accordingly. For example, increased administration of the agent may be desirable to decrease the level of a ganglioside to lower levels than detected, i.e., to increase the effectiveness of the agent. According to such an embodiment, a ganglioside may be used as an indicator of the effectiveness of an agent, even in the absence of an observable phenotypic response. Similarly, a ganglioside analysis, such as by immunohistochemistry (IHC) or by a mass spectrometry-based methods, can also be used to select patients who will receive a cancer therapy (e.g., immunotherapy, immune checkpoint inhibition therapy).

### Sample

Biological samples can be collected from a variety of sources from a subject including a body fluid sample, cell sample, or a tissue sample. Body fluids refer to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In some embodiments, the subject and/or control sample is selected from the group consisting of cells, cell lines, whole blood, serum, plasma, buccal scrape, saliva, cerebrospinal fluid, and bone marrow. In some embodiments, samples can contain live cells/tissue, fresh frozen cells, fresh tissue, biopsies, fixed cells/tissue, cells/tissue embedded in a medium, such as paraffin, histological slides, or any combination thereof.

The samples can be collected from individuals repeatedly over a longitudinal period of time (e.g., once or more on the order of days, weeks, months, annually, biannually, etc.).

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids. In some embodiments, certain cell types are purified based on at least one marker present on the cell surface.

A sample may comprise a fixed molecule. A molecule is “fixed” or “affixed” to a substrate if it is covalently or non-covalently associated with the substrate such the substrate can be rinsed with a fluid (e.g. standard saline citrate, pH 7.4) without a substantial fraction of the molecule dissociating from the substrate.

As described herein, in some embodiments, the level of at least one ganglioside measurement(s) in a sample from a subject is compared to a control biological sample (e.g., biological sample from a subject who does not have a cancer), a control biological sample from the subject during remission or before developing a cancer, or a control biological sample from the subject during treatment for developing a cancer. In some embodiments, a control biological sample is from a subject prior to treatment with a certain therapy. In some embodiments, wherein a subject is treated with multiple rounds of one or more therapies, a control biological sample may be from an earlier or later time point with respect to the subject sample during such treatment. For example, a subject sample after third rounds of therapy may be compared with a control subject sample after the first round of therapy.

In some embodiments, the level of at least one ganglioside measurement(s) in a sample from a subject is compared to a predetermined control (standard) sample. The sample from the subject is typically from a diseased tissue, such as cancer cells or tissues. The control sample can be from the same subject or from a different subject. The control sample is typically a normal, non-diseased sample. However, in some embodiments, such as for staging of disease or for evaluating the efficacy of treatment, the control sample can be from a diseased tissue. The control sample can be a combination of samples from several different subjects. In some embodiments, the biomarker amount and/or activity measurement(s) from a subject is compared to a pre-determined level. This pre-determined level is typically obtained from normal samples.

As described herein, a “pre-determined” biomarker amount measurement(s) may be a biomarker amount measurement(s) used to, by way of example only, evaluate a subject that may be selected for treatment, evaluate a response to a cancer therapy, and/or evaluate a response to a combination of anti-cancer therapies. A pre-determined biomarker amount and/or activity measurement(s) may be determined in populations of patients with or without cancer. The pre-determined biomarker amount measurement(s) can be a single number, equally applicable to every patient, or the pre-determined biomarker amount measurement(s) can vary according to specific subpopulations of patients. Age, weight, height, and other factors of a subject may affect the pre-determined biomarker amount measurement(s) of the individual. Furthermore, the pre-determined biomarker amount can be determined for each subject individually. In some embodiments, the amounts determined and/or compared in a method described herein are based on absolute measurements.

In some embodiments, the presence or the level of at least one ganglioside measurement(s) in a sample from a subject is compared with the sample without an agent that detects the ganglioside. For example, if an antibody or antigen-binding fragment thereof is used to detect the level of a ganglioside in a subject sample, the control sample for comparison may be the same subject sample in which the antibody or antigen-binding fragment thereof is omitted, i.e., “background signal.” Such background signal control is used in experimental data presented herein.

In some embodiments, the amounts determined and/or compared in a method described herein are based on relative measurements, such as ratios (e.g., biomarker level before a treatment vs. after a treatment, such biomarker measurements relative to a spiked or man-made control, such biomarker measurements relative to the expression of a housekeeping gene, and the like). For example, the relative analysis can be based on the ratio of pre-treatment biomarker measurement as compared to post-treatment biomarker measurement. Pre-treatment biomarker measurement can be made at any time prior to initiation of anti-cancer therapy. Post-treatment biomarker measurement can be made at any time after initiation of anti-cancer therapy. In some embodiments, post-treatment biomarker measurements are made 1, 2, 3, 4, 5, 6, 7, 8, 9, 10, 11, 12, 13, 14, 15, 16, 17, 18, 19, 20 weeks or more after initiation of anti-cancer therapy, and even longer toward indefinitely for continued monitoring. Treatment can comprise one or more anti-cancer therapies, e.g., immune checkpoint inhibitors.

The pre-determined biomarker amount measurement(s) can be any suitable standard. For example, the pre-determined biomarker amount measurement(s) can be obtained from the same or a different human for whom a patient selection is being assessed. In some embodiments, the pre-determined biomarker amount measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

In some embodiments of the present disclosure the change of biomarker amount measurement(s) from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cutoff values apply equally when the measurement is based on relative changes, such as based on the ratio of pre-treatment biomarker measurement as compared to post-treatment biomarker measurement.

### Cancer

Cancer, tumor, or hyperproliferative disorder refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma (SCLC), bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

The compositions and methods of the present invention may be used to detect ovarian cancer, small cell lung cancer (SCLC), or melanoma.

### Cancer Therapy

The therapeutic agents of the present invention can be used alone or can be administered in combination therapy with, e.g., chemotherapeutic agents, hormones, antiangiogens, radiolabelled, compounds, or with surgery, cryotherapy, immunotherapy, cancer vaccine, immune cell engineering (e.g., CAR-T), and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, agents of the present invention can be administered with a therapeutically effective dose of chemotherapeutic agent. In other embodiments, agents of the present invention are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer being treated, the extent of the disease and other factors familiar to the physician of skill in the art, and can be determined by the physician.

Immunotherapy is a targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF is known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy also encompasses immune checkpoint modulators. Immune checkpoints are a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, TMIDG2, KIR3DL3, and A2aR (see, for example, WO 2012/177624). Inhibition of one or more immune checkpoint inhibitors can block or otherwise neutralize inhibitory signaling to thereby upregulate an immune response in order to more efficaciously treat cancer. In some embodiments, the cancer vaccine is administered in combination with one or more inhibitors of immune checkpoints (immune checkpoint inhibition therapy), such as PD1, PD-L1, and/or CD47 inhibitors.

Adoptive cell-based immunotherapies can be combined with the therapies of the present invention. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, and the like.

The term “chimeric antigen receptor” or “CAR” refers to engineered T cell receptors (TCR) having a desired antigen specificity. T lymphocytes recognize specific antigens through interaction of the T cell receptor (TCR) with short peptides presented by major histocompatibility complex (MHC) class I or II molecules. For initial activation and clonal expansion, naïve T cells are dependent on professional antigen-presenting cells (APCs) that provide additional co-stimulatory signals. TCR activation in the absence of co-stimulation can result in unresponsiveness and clonal anergy. To bypass immunization, different approaches for the derivation of cytotoxic effector cells with grafted recognition specificity have been developed. CARs have been constructed that consist of binding domains derived from natural ligands or antibodies specific for cell-surface components of the TCR-associated CD3 complex. Upon antigen binding, such chimeric antigen receptors link to endogenous signaling pathways in the effector cell and generate activating signals similar to those initiated by the TCR complex. Since the first reports on chimeric antigen receptors, this concept has steadily been refined and the molecular design of chimeric receptors has been optimized and routinely use any number of well-known binding domains, such as scFV and another protein binding fragments described herein.

In other embodiments, immunotherapy comprises non-cell-based immunotherapies. In some embodiments, compositions comprising antigens with or without vaccine-enhancing adjuvants are used. Such compositions exist in many well-known forms, such as peptide compositions, oncolytic viruses, recombinant antigen comprising fusion proteins, and the like. In some embodiments, immunomodulatory cytokines, such as interferons, G-CSF, imiquimod, TNFalpha, and the like, as well as modulators thereof (e.g., blocking antibodies or more potent or longer lasting forms) are used. In some embodiments, immunomodulatory interleukins, such as IL-2, IL-6, IL-7, IL-12, IL-17, IL-23, and the like, as well as modulators thereof (e.g., blocking antibodies or more potent or longer lasting forms) are used. In some embodiments, immunomodulatory chemokines, such as CCL3, CCL26, and CXCL7, and the like, as well as modulators thereof (e.g., blocking antibodies or more potent or longer lasting forms) are used. In some embodiments, immunomodulatory molecules targeting immunosuppression, such as STAT3 signaling modulators, NFkappaB signaling modulators, and immune checkpoint modulators, are used.

In still other embodiments, immunomodulatory drugs, such as immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-xB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), 13C (indole-3-carbinol)/DIM (di-indolmethane) (13C/DIM), Bay 11-7082, luteolin, cell permeable peptide SN-50, IKBa.-super repressor overexpression, NFKB decoy oligodeoxynucleotide (ODN), or a derivative or analog of any thereo, are used. In yet other embodiments, immunomodulatory antibodies or protein are used. For example, antibodies that bind to CD40, Toll-like receptor (TLR), OX40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 a antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL12 inhibitor, an IL23 inhibitor, ustekinumab, and the like.

Nutritional supplements that enhance immune responses, such as vitamin A, vitamin E, vitamin C, and the like, are well-known in the art (see, for example, U.S. Pat. Nos. 4,981,844 and 5,230,902 and PCT Publ. No. WO 2004/004483) can be used in the methods described herein.

Similarly, various agents or a combination thereof can be used to treat a cancer. For example, chemotherapy, radiation, epigenetic modifiers (e.g., histone deacetylase (HDAC) modifiers, methylation modifiers, phosphorylation modifiers, and the like), targeted therapy, and the like are well-known in the art.

In some embodiments, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6 (5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of.beta.-nicotinamide adenine dinucleotide (NAD+) into nicotinamide and poly-ADP-ribose (PAR). Both poly(ADP-ribose) and PARP have been linked to regulation of transcription, cell proliferation, genomic stability, and carcinogenesis (Bouchard V. J. et.al. Experimental Hematology, Volume 31, Number 6, June 2003, pp. 446-454 (9); Herceg Z.; Wang Z.-Q. Mutation Research/Fundamental and Molecular Mechanisms of Mutagenesis, Volume 477, Number 1, 2 Jun. 2001, pp. 97-110 (14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In other embodiments, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In other embodiments, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In other embodiments, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light.

In yet other embodiments, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors.

### Clinical Efficacy/Response to a Therapy

Clinical efficacy can be measured by any method known in the art. For example, the response to a therapy relates to any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al., J. Clin. Oncol. (2007) 25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular anti-immune checkpoint therapeutic regimen is at least 25%, 30%, 35%, 40%, 45%, 50%, 55%, 60%, 65%, 70%, 75%, 80%, 85%, or more.

Additional criteria for evaluating the response to a cancer therapy are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include probability of survival, probability of metastasis within a given time period, and probability of tumor recurrence.

For example, in order to determine appropriate threshold values, a particular anti-cancer therapeutic regimen can be administered to a population of subjects and the outcome can be correlated to biomarker measurements that were determined prior to administration of any cancer therapy. The outcome measurement may be pathologic response to therapy given in the neoadjuvant setting. Alternatively, outcome measures, such as overall survival and disease-free survival can be monitored over a period of time for subjects following the cancer therapy for whom biomarker measurement values are known. In certain embodiments, the same doses of anti-cancer agents are administered to each subject. In related embodiments, the doses administered are standard doses known in the art for anti-cancer agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy can be determined using methods such as those described in the Examples section.

### Kits

The present invention also encompasses kits for detecting the presence or the level of a ganglioside in a biological sample. For example, the kit can comprise a labeled compound or agent capable of detecting a ganglioside in a biological sample; means for determining the amount of the ganglioside in the sample; and means for comparing the amount of the ganglioside in the sample with a standard. The compound or agent can be packaged in a suitable container. For example, in some embodiments, the present invention provides kits comprising at least one antibody, or an antigen-binding fragment thereof, described herein. Kits containing antibodies, or antigen-binding fragments thereof, of the invention find use in detecting a ganglioside, e.g., in diagnostic or prognostic assays. Kits of the present invention can contain an antibody, or an antigen-binding fragment thereof, coupled to a solid support, e.g., a tissue culture plate or beads (e.g., sepharose beads).

A kit can include additional components to facilitate the particular application for which the kit is designed. For example, kits can be provided which contain antibodies for detection and quantification of a ganglioside in vitro or ex vivo, e.g. in an ELISA or a Western blot. Additional, exemplary agents that kits can contain include means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or a ganglioside standards). A kit may additionally include buffers and other reagents recognized for use in a method of the disclosed invention. Non-limiting examples include agents to reduce non-specific binding, such as a carrier protein or a detergent. A kit of the present invention can also include instructional materials disclosing or describing the use of the kit or an antibody of the disclosed disclosure in a method of the disclosed disclosure as provided herein.

This invention is further illustrated by the following examples which should not be construed as limiting. The contents of all references, patents and published patent applications cited throughout this application, as well as the Figures, are incorporated herein by reference.

### EXAMPLES

### Example 1: Materials and Methods

### Mice

The animal protocol used was reviewed and approved by the Lady Davis Institute Animal Care Committee and animal experiments were performed according to the guidelines of the Canadian Council on Animal Care. Healthy wild-type female C₅₇/B16 mice (10-12 weeks of age, 19-20 g) were purchased from Harlan (Lachine, Quebec, Canada) and used to immunize with PAMAM-GD2 and PAMAM-GD3 to generate antibodies. A maximum of five mice per cage were kept in a 12-h dark-light cycle with food and water ad libitum.

### Immunization

PAMAM-GD2 and PAMAM-GD3 were each injected twice intraperitoneally (50 ug in PBS) to mice about 5-7 days apart. The first immunization had 10% (v/v) gerbu (an adjuvant), while the second immunization had no adjuvant. Splenocytes were harvested from the immunized mice and fused with myeloma lines to generate hybridomas (Haskard and Archer, J. Immunol. Methods, 74 (2), 361-67 (1984), Roder et al., Methods Enzymol., 121, 140-67 (1986), and Huse et al., Science, 246, 1275-81 (1989)). The ability of the antibodies to bind to PAMAM-GD2, PAMAM-GD3, or to native gangliosides was analyzed by flow cytometry and ELISA.

### Immunohistochemistry (IHC)

Paraffin-embedded 4-μm-thick tissue sections were deparaffinized and washed in phosphate buffer saline (PBS). Endogenous hydrogen peroxidase and biotin were blocked with 0.3% (v/v) H₂O₂ and Avidin/Biotin blocking kit (Vector Laboratories, SP-2001), respectively. Unspecific background was blocked with Blocking reagent followed by overnight incubation with an anti-GD2 antibody or an anti-GD3 antibody. Sections were incubated with anti-biotynilated mouse IgG horse rabbit peroxidase (HRP), followed by DAB reaction, and counterstaining with Hematoxylin/Eosin. Sections without primary antibody were used as negative control. Images were taking using a light microscope scanner.

### Scoring

The immunoreactivity of GD2 and GD3 was reviewed and scored using a semi-quantitative method by a pathologist (Ziebarth et al, 2012: Uterine leiomyosarcoma diffusely express disialoganglioside GD2 and bind the therapeutic immunocytokine 14.18-IL2: Implications for immunotherapy) and Orsi et al, 2017: GD2 expression in breast cancer). The immunostaining was assessed in a blind manner and independently from the clinicopathological data. The samples were classified according to their intensity into different categories. No immunoreactivity (0), 1+ (weak stain), 2+ (moderate stain), 3+ (strong stain). Those samples with scores 0 or 1+ stain were considered negative and scores 2+ and 3+ were considered positive. Final GD2 or GD3 scores were shown as percentage (%) of total ovarian samples or of cancer ovarian subtypes.

### ELISA

Gangliosides were isolated from equal volume of serum from each donor, using organic solvent extraction methods (see e.g., Example 10). Serum-equivalent volumes of analytes were immobilized in ELISA plates, and assayed with mAbs against GD2 or GD3. After incubation with secondary antibody, optical density (OD) was read at 450 nm. Negative values were set at optical density (OD)<0.15; low positive GD2 values at OD between 0.15-0.5 and high positive GD2 values at OD>0.5. Control purified native GD2 or GD3 (10 ng/well) were used as standard positive control. Wells with no primary were used as background. Each sample was assayed in triplicate, and each ELISA replicated at least 2-3 independent times.

### Flow Cytometry

2×10⁵ cells of EL4-GD2+, EL4-GD3+, and Jurkat cells were incubated for 20 min on ice with 2 mL mouse antisera (1:50 dilution) or with positive control anti-GD2 mAb (13 nM, 14G2a; Santa Cruz Biotechnology) or positive control anti-GD3 mAb (13 nM, R24; Abcam), followed by FITC-conjugated anti-mouse IgG secondary (1.8 nM, Sigma). Cells were studied immediately in a flow cytometer (Becton-Dickinson), and data analyzed using CellQuest software. Pre-bleed sera and normal mouse sera were used as negative controls. Jurkat cells (negative for GD2 and GD3) were used as negative control cells.

### Patients

Patients samples (TMAs and liquid biopsies) were obtained from the LDI/JHG biobank under ethical approval. Retrospective study of clinical history was used to asses the correlation between TMGs and CA-125 levels, cancer disease stages and survival, and menopausal stage. A total of 176 patients were studied (n=9 normal; n=16 borderline; n=151 ovarian). From those, the levels of GD2 were analyzed in liquid biopsies by ELISA and compared to CA-125 values in the same patients (n=40 ovarian patients; n=7 borderline; n=33 ovarian).

### Example 2: Design and Synthesis of Carbohydrate Analogs and Dendrimer Products for Generation of Antibodies Against GD2 and GD3

As described herein, upregulation of gangliosides are prevalent in tumors.

TABLE 3
Matrix of cancers with upregulated tumor marker gangliosides

| | | | | | | | Sialyl | Sialyl |
|---|---|---|---|---|---|---|---|---|
| | | | Fucosyl- | | | | Lewis | Lewis |
| Indication | GD2 | GD3 | GM1 | GM2 | GM3 | PolySia | X | A |
| Neuroblastoma | X | X | X | X | X | X | | |
| Lymphoma | X | X | X | X | X | | | |
| Leukemia | X | X | | | | | | |
| Melanoma | X | X | | | | | | |
| Glioma | X | X | | X | X | X | | |
| Small Cell Lung | X | X | X | X | | | | |
| Cancer | | | | | | | | |
| Breast Carcinoma | X | X | | X | X | X | X | X |
| Ovarian Cancer | X | X | X | X | X | | | |
| Soft Tissue | X | X | | | | | | |
| Sarcomas | | | | | | | | |
| Osteosarcoma | X | X | | | | | | |
| Ewing's Sarcoma | X | X | | | X | | | |
| Desmoplastic | X | X | | | | | | |
| Round Cell Tumor | | | | | | | | |
| Rhabdomyosarcoma | X | X | | | | | | |
| Retinoblastoma | X | | | | X | | | |
| Non-Small Cell | | | X | X | X | X | X | X |
| Lung Cancer | | | | | | | | |
| Renal Cell Cancer | | | X | X | X | | X | X |
| Wilms Tumor | | | | | X | X | | |
| (Nephroblastoma) | | | | | | | | |
| Medullary Thyroid | | | | | | X | | |
| Cancer | | | | | | | | |
| Prostate Cancer | | | | X | | | | |
| Gastric Cancer | | | | X | | X | X | X |
| Endometrial Cancer | | | | X | | X | | |
| Pancreatic Cancer | | | | X | | X | X | X |
| Colon Cancer | | | | X | | | X | X |
| Esophageal Cancer | | | | | | | X | X |
| Head and Neck | | | | | | | X | X |

The carbohydrates of normal gangliosides and TMGs share common structures. For example, GD2 and GD3 differ from each other by one sugar, and the ubiquitously expressed GM1 differs from GD2 by two sugar units. Hence, it is very difficult to safely raise anti-ganglioside immunity. It is prepared herein ganglioside mimetics that are structurally identical to native GD2 and GD3 carbohydrates and without the variable lipid tail, to reduce heterogeneity and the risk of cross-reactivity. Synthetic analogs p-amino phenyl ether-GD2 (AP-GD2) and p-amino phenyl ether-GD3 (AP-GD3) were generated, whereby their amine group can be exploited for conjugation to amenable scaffolds. In AP-GD2 and AP-GD3, the anomeric center of the glucose linked to the phenyl ether is in the b-configuration, which is the native linkage found between sugar and ceramide in the natural gangliosides. This stereochemistry is key for preserving the native structure, and contrasts with some chemistry-only based synthetic methods that result in a mixture of a- and b-configurations. After high-performance liquid chromatography purification, the identity of AP-GD2 was verified by liquid chromatography-mass spectrometry, and the configuration was confirmed using 1H nuclear magnetic resonance (NMR) spectroscopy. The AP-GD3 was characterized similarly.

Using AP-GD2 and AP-GD3 glycomimetic precursors, the lipid-free, water-soluble oligomeric products were synthesized to immunize mice for generation of the antibodies. Purified AP-GD2 and AP-GD3 were converted to the corresponding isothiocyanate and coupled to the free amines of PAMAM GO dendritic core to provide the corresponding thioureas, PAMAM-GD2 and PAMAM-GD3. After separation from unreacted precursors and excess reagents, PAMAM-GD2 and PAMAM-GD3 were characterized using thin-layer chromatography, NMR spectroscopy, and quantitative. Additional details regarding the synthesis are provided below.

### Synthesis of AP-GD2 and AP-GD3

p-Aminophenylether-b-D-lactopyranoside (AP-Lac, from Toronto Research Chemicals) was used to enzymatically synthesize water soluble (>20 mg/ml) AP-GD2 and AP-GD3 intermediate carbohydrates. The synthesis of AP-GD2 included 215 mmol of AP-GD3 (4 mM), 295 mmol of UDP-GalNAc (5.4 mM), and 10.5 units of CgtA (construct CJL-30), 10 mM MnCl₂ and 24 mM Hepes pH 7.0 in a final volume of 55 ml. The reaction mixture was incubated at 37° C. for 6.5 hr. Insoluble material was removed by centrifugation at 27,000 g for 15 min and the enzyme was removed by ultrafiltration using a 10 kDa membrane cut off. The filtrate was loaded on SepPak C18 5 g column (waters Corp) and the product was eluted with water while unidentified contaminants remained in the column. The product was further purified by anion exchange chromatography on HiTrapQ (GE Healthcare) using a gradient of 0-0.3 M NH₄HCO₃ and by size exclusion chromatography on a Superdex Peptide 10/300 GL column (GE Healthcare). The measured molecular weights of AP-GD2 (1,218 g/mol) and AP-GD3 (927 g/mol) correspond to expected values. Structures were verified by 1D and 2D NMR spectroscopy and mass spectrometry (EI-MS). The AP-GD2 and AP-GD3 intermediates were purified to >99% purity by size-exclusion chromatography (Superdex 30 16 mm×85 cm column, GE Health Care) before their application in the synthesis of vaccines by conjugation to a dendrimer scaffold.

### Synthesis of PAMAM-GD2 and PAMAM-GD3 Dendrimers

Thiophosgene (2 ml) was added to a stirred solution of AP-GD2 (2 mg) in 80% ethanol (300 ml). After 3 hr at room temperature, thin layer chromatography (ethyl acetate: methanol: water: acetic acid 4:2:1:0.1 v/v) showed that a single product had formed. Concentration to near dryness gave a solid, which was treated with water and filtered. The filter cake was washed with water, and the combined filtrate and washings were freeze-dried to give isothiocyanatophenyl GD2 as white powder (1.8 mg, 90% yield). In a separate flask, the volatiles from a methanol solution of polyamidoamine (PAMAM GO, Dendritech, Inc) were evaporated under reduced pressure, and the resulting residue was dissolved in dimethylformamide (DMF). Isothiocyanatophenyl GD2 (1.8 mg) in DMF (110 ml) was added drop-wise to a stirred DMF solution (100 ml) of N,N-diisopropylethylamine (0.5 ml) and PAMAM GO (2 ml of 85.4 mg/ml). The reaction was stirred at room temperature for 20 h, until no starting material was detected by TLC. The mixture was diluted with water and dialyzed against water (MW cutoff 2 kDa, Spectrum Laboratories Inc.). The resulting solution was freeze-dried to give 1.34 mg (80% yield) of PAMAM-GD2 product as white powder that was further characterized by 1D and 2D NMR spectroscopy. The PAMAM-GD3 was synthesized from AP-GD3 precursor and PAMAM using a similar process. Syntheses of PAMAM-GD2 and PAMAM-GD3 were reproduced each at least three times, yielding identical products as determined by TLC and NMR.

The products are heterogeneous mixtures of different amounts of GD2 moieties conjugated onto PAMAM, ranging from 0-4 and including a fully conjugated tetramer (e.g. see the aromatic region (6 ppm and above) of the 1H NMR). Thus, further investigation of the components of the products was undertaken. The HPLC of PAMAM-GD2 showed two major peaks which were isolated (peak 1 and peak 2) and subjected to NMR analysis. Peak 1 is a PAMAM-lactose tetramer, having lost the GalNAc and sialic acid residues and which does not contain GD2 epitopes. Peak 2 is a mixture PAMAM-GD2 with all its components, and contains GD2 epitopes including a tetramer. Quantification of the amount of antigen (AP-GD2) in PAMAM-GD2 was done by using a selective anti-GD2 mAb (clone 14G2a; Santa Cruz Biotechnology). The amount (w/w) of GD2-reactive epitopes were quantified in each of the different components of PAMAM-GD2 mixtures. The loss of sialic acid and GalNAc residue in peak 1 results in no detection of GD2 epitopes. All other fractions isolated from PAMAM-GD2 mixtures bind to 14G2a (Santa Cruz Biotechnology), suggesting that those fractions have GD2 content. Quantification from dose-response curves indicates that 49±12% (w/w) is AP-GD2 content in the PAMAM-GD2 mixture.

### Example 3: Design and Synthesis of the Modified Versions of Gangliosides

Exemplary species of modified gangliosides were made by coupling a heteroaryl (e.g., triazine) with various forms of GD2 or GD3 (e.g., amino-phenyl GD, amino-propyl GD, aminoethyl GD, aminobutyl GD, etc.). Detailed methods of synthesis are provided below.

Synthesis of Triazine-GD2 conjugate (Tria-tri-GD2)

2-Amino-4,6-dichloro-1,3,5-triazine (1 mg, 0.007 mmol) was dissolved in 1 mL of anhydrous DMF. To that solution was added powdered 1,1-thiocarbonyldiimidazole (2.5 mg, 0.014 mmol) and the reaction mixture was stirred for 3 h. The progress of the reaction was monitored by HPLC (eluting gradient solvent system used was 0 to 100% acetonitrile in water). The peak with retention time 3.54 min (2-Amino-4,6-dichloro-1,3,5-triazine, starting material and limiting reactant)disappeared and a new peak with retention time at 2.29 min was generated. The peak at the retention time of 2.29 min was isolated and was lyophilized. The product was used for the next step without any further purification or characterization.

To the product (0.1 mg, 0.533 μmol) in 1 mL DMF was added aminophenylGD2 (3.2 μmol, 4 mg) and 20 μL of 1 N NaOH was added in the reaction mixture. The reaction mixture was then stirred for 12 h and the progress of the reaction was monitored by HPLC using the same gradient solvent system (0 to 100% acetonitrile in water). The peak at 2.29 min disappeared and a new major peak at 1.89 min appeared.

This peak was isolated, lyophilized and used for activity assays (see ELISA).

Synthesis of Triazine-GD3 conjugate (Tria-tri-GD3)

2-Amino-4,6-dichloro-1,3,5-triazine (1 mg, 0.007 mmol) was dissolved in 1 mL of anhydrous DMF. To that solution was added powdered 1,1-thiocarbonyldiimidazole (2.5 mg, 0.014 mmol) and the reaction mixture was stirred for 3 h. The progress of the reaction was monitored by HPLC (eluting gradient solvent system used was 0 to 100% acetonitrile in water). The peak with retention time 3.54 min (2-Amino-4,6-dichloro-1,3,5-triazine, starting material and limiting reactant)disappeared and a new peak with retention time at 2.29 min was generated. The peak at the retention time of 2.29 min was isolated and was lyophilized. The product was used for the next step without any further purification or characterization.

To the product (0.1 mg, 0.533 μmol) in 1 mL DMF was added aminophenylGD3 (3.2 μmol, 3.2 mg) and 20 μL of 1 N NaOH was added in the reaction mixture. The reaction mixture was then stirred for 12 h and the progress of the reaction was monitored by HPLC using the same gradient solvent system (0 to 100% acetonitrile in water). The peak at 2.29 min disappeared and a new major peak at 2.34 min appeared.

This peak was isolated, lyophilized and used for activity assays (see ELISA).

Synthesis of analogs of Triazine-GD2 with x=2 stoichiometry (Tria-di-GD2)

2-Amino-4,6-dichloro-1,3,5-triazine (1 mg, 0.007 mmol) was dissolved in 1 mL of anhydrous DMF. 10 mg of solid sodium carbonate was added to it. To that solution was added powdered Boc-anhydride (3 mg, 0.014 mmol) and the reaction mixture was stirred for 12 h. The progress of the reaction was monitored by HPLC (eluting gradient solvent system used was 0 to 100% acetonitrile in water). The peak with retention time 3.54 min (2-Amino-4,6-dichloro-1,3,5-triazine, starting material and limiting reactant)disappeared and a new peak was generated. The peak was isolated and lyophilized. The product was used for the next step without any further purification or characterization.

To the product (0.1 mg) in 1 mL DMF was added aminophenyl-GD2 (3.2 μmol, 4 mg) and 20 μL of 1 N NaOH was added in the reaction mixture. The reaction mixture was then stirred for 12 h and the progress of the reaction was monitored by HPLC using the same gradient solvent system (0 to 100% acetonitrile in water). The peak at 2.29 min disappeared and a new major peak at 1.89 min appeared.

Removal of the BOC protecting group is optional, and if the group is removed the deprotected site can be derivatized with fluorochromes (e.g. FITC or Cy7) or tags, such as biotin, using commercially available reagents.

### Synthesis of Triazine-di-GD3

2-Amino-4,6-dichloro-1,3,5-triazine (1 mg, 0.007 mmol) was dissolved in 1 mL of anhydrous DMF. To that solution was added powdered 1,1-thiocarbonyldiimidazole (2.5 mg, 0.014 mmol) and the reaction mixture was stirred for 3 h. The progress of the reaction was monitored by HPLC (eluting gradient solvent system used was 0 to 100% acetonitrile in water). The peak with retention time 3.54 min (2-Amino-4,6-dichloro-1,3,5-triazine, starting material and limiting reactant)disappeared and a new peak with retention time at 2.29 min was generated. The peak at the retention time of 2.29 min was isolated and was lyophilized. The product was used for the next step without any further purification or characterization.

To the product (0.1 mg, 0.533 μmol) in 1 mL DMF was added aminophenylGD3 (3.2 μmol, 3.2 mg) and 20 μL of IN NaOH was added in the reaction mixture. The reaction mixture was then stirred for 12 h and the progress of the reaction was monitored by HPLC using the same gradient solvent system (0 to 100% acetonitrile in water). The peak at 2.29 min disappeared and a new major peak at 2.34 min appeared.

This peak was isolated, and lyophilized. Removal of the BOC protecting group is optional, and it the group is removed the deprotected site can be derivatized with fluorochromes (e.g. FITC or Cy7) or tags such as biotin, using commercially available reagents.

Synthesis of FITC labelled Triazine-di-GD2

To the boc-protected Triazine-diGD2 in water, was added 20% (v/v) in acetic acid. The reaction mixture was stirred at room temperature for 1 h. The progress of the reaction was monitored by HPLC (eluting gradient solvent system used was 0 to 100% acetonitrile in water). The product was used for the next step without any further purification.

To the product in DMF was added fluorescein isothiocyanate (FITC) and the reaction was carried out for 12 h. The progress of the reaction was monitored by HPLC (eluting gradient solvent system used was 0 to 100% acetonitrile in water).

Synthesis of FITC labelled Triazine-di-GD3

To the boc-protected Triazine-diGD3 (1 mg) solution in water, (1 mL) was added 20% (v/v) acetic acid. The reaction mixture was stirred at room temperature for 1 h. The progress of the reaction was monitored by HPLC (eluting gradient solvent system used was 0 to 100% acetonitrile in water). The peak with boc-protected Triazine-diGD3 retention time disappeared and a new peak was generated, isolated, and was lyophilized. The product was used for the next step without any further purification or characterization.

To the product in DMF was added 3-molar excess fluorescein isothiocyanate and the reaction was carried out for 12 h. The progress of the reaction was monitored by HPLC (eluting gradient solvent system used was 0 to 100% acetonitrile in water), and a new peak was generated, isolated, and lyophilized.

The pure product was then used for activity and for binding studies.

Generation of Anti-human GD2 and GD3 antibodies

From immunization of mice with PAMAM-GD2 and PAMAM-GD3, twelve unique hybridomas secreting selective anti-GD2 or anti-GD3 mAbs were generated herein. This is a major feat, because worldwide there are only a handful of selective mAbs. This is because carbohydrates have poor immunogenicity, thus it is difficult to generate antibodies that specifically bind to carbohydrates, let alone carbohydrate portions of gangliosides such as GD2 and GD3. The mAbs presented herein bind to the unique carbohydrate portion of GD2 or GD3 and are highly selective. In flow cytometry assays, each mAb binds only to the cell surface of cell lines expressing GD2 or expressing GD3 (mouse rat or human) but there is no binding to cells known to be negative for GD2 or for GD3. The binding and selectivity of the 12 mAbs have been characterized by flow cytometry. For IHC and ELISA, four of the 12 distinct mAbs (Clones disclosed in Tables 1 and 2) have been characterized in depth, because these mAbs are also cytotoxic to tumors and useful for cancer immunotherapy in vivo.

### Example 4: Quantification of GD2 and GD3 in Human Liquid Biopsies by ELISA

Knowing that GD2 and GD3 are shed from cancer cells, an ELISA method was applied that was developed for gangliosides and evaluated the presence of GD2 or GD3 in fifty ovarian cancer patient blood samples (n=37 diagnosed high grade and n=13 diagnosed early stage or borderline). In blinded ELISA studies 48/50 (96%) and 46/50 (92%) were positive for GD2 or GD3. The overall rate was 49/50 (98%). All liquid biopsies from healthy and non-cancer patients were negative and had values equal to background (n=23 individual non-cancer females; and one pooled sample of 30 healthy donors).

### Longitudinal Evaluation of Liquid Biopsy Monitors Response or Recurrence.

A longitudinal evaluation of GD2 and GD3 in blood samples correlated detection with tumor burden and response to treatment. Sera were collected before therapy (at diagnosis), and ˜2 weeks after completion of treatment (surgery plus adjuvant chemotherapy). Tumor volumes were quantified by PET imaging (data show cumulative tumor volumes in the whole body; Table 4).

TABLE 4
Liquid biopsies of melanoma patients collected
before and after standard therapy

| | Patient 1 (responsive) | | Patient 2 (non-responsive) | |
|---|---|---|---|---|
| | Treatment | | Treatment | |
| | naïve | Post-therapy | naïve | Post-therapy |
| | initial | residual | initial | residual |
| Analyte | 310 cm³ | 3.1 cm³ | 183 cm³ | 103 cm³ |
| GD3 | 22 | 2 | 20 | 13 |
| GD2 | 2 | 1 | 2 | 2 |
| PS control | 15 | 29 | 7 | 26 |

* Blood GD2 or GD3 is found in patinets with melanoma tumors.
* GD2 or GD3 levels in blood correlate with tumor volume.
*Reduced tumor volume (post-therapy) results in reduced GD2 or GD3 levels in blood.

Two patients are shown as examples. Patient 1: tumor burden at diagnosis was 310 cm³ and GD2/GD3 levels were 10-fold elevated above normal. The patient responded well to treatment. After therapy residual tumor was 3.0 cm³ and GD2/GD3 levels were significantly reduced and indistinguishable from normal. Patient 2: tumor burden at diagnosis was 183 cm³ and GD2/GD3 levels were 6-fold elevated above normal. The patient responded poorly to treatment. After therapy, residual tumor remained large at 103 cm³, and the GD2/GD3 levels remained high, with a minor 30% reduction in the GD3 level.

The results demonstrated that the ELISA is useful to monitor treatment response or cancer recurrence. In particular, the limit of quantitative detection (LOQD) is equivalent to minimal residual disease detectable by metabolic PET, which would make this test valuable for the monitoring of cancer recurrence. Glycolipids are extracted in organic solvents from 1 ml of serum. Each sample is assayed in triplicate, and each ELISA replicated at least 2-3 independent times, using two independent mAbs for each GD2 or GD3. Only 1 ml of serum is sufficient to test a sample and to cross-validate it by other methods. The rest of a standard blood collection tube (5 ml) may be used for evaluation of other markers; and for archival purposes. A standard curve of purified native GD2 or GD3 are used as internal positive control, normal ganglioside GM1 is used as negative control (10 ng/well), and a range of mAb concentrations (225 nM, 75 nM, 25 nM) are used. Background wells have all reagents but no primary mAb. As cut-off, a statistically significant 2-fold higher value from the average of all controls, with at least 2x sd from controls is used.

### Example 5: Detection of GD2 and GD3 in Ovarian Cancer Tissue Biopsies by IHC

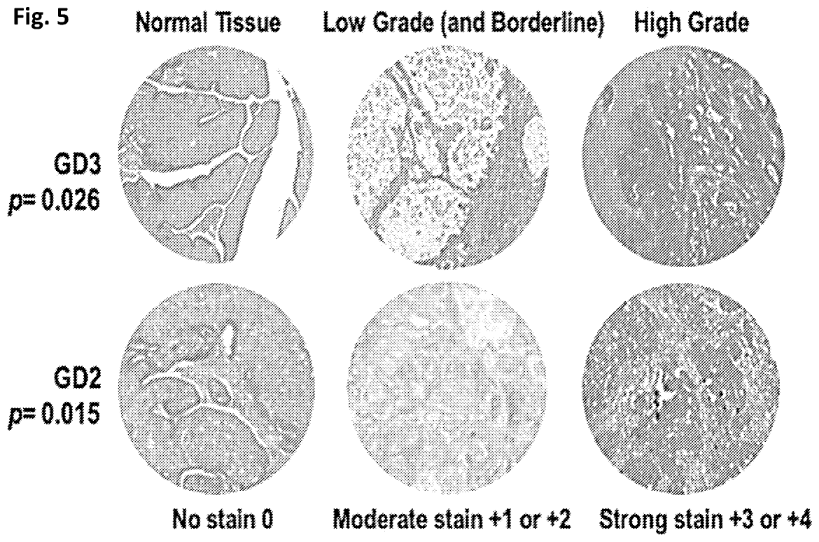

Anti-GD2 and anti-GD3 mAbs were evaluated in IHC of 113 ovarian cancer tissue biopsies (75 advanced stage and 38 low stage). As used herein, cancer at the early/low stage corresponds to cancer at Stage I; and cancer at the late/high/advanced stage includes cancer at Stage II to Stage IV. Overall 109/113 (96%) were positive for GD2 and GD3; with 73/75 at advanced stage and 36/38 at early stage. Staining was significant and homogeneous, restricted to tumor tissue, and absent in the normal surrounding tissue (FIG. 4). It is key to note that the tissues that were studied by IHC comprise the same 50 patients whose blood was used in ELISA (see Example 5). The data correlated 100%. This is cross-validating, since a blood-borne GD2 can only arise from a GD2-positive tumor.

A comparison with the standard CA-125 marker (detectable in ˜60% early stage and ˜85% late stage, and not useful for monitoring early recurrence) showed that GD2/GD3 were a significantly superior marker. For early stage patients 25/38 had abnormal levels of CA-125 (244±106 U/1) and 24 of those 25 were positive for GD2. The other 13/38 early stage patients had normal CA-125 levels and 12 of those 13 were GD2+ive. Hence the ELISA would have detected 12/13 early stage patients that otherwise would not be detected. Overall the early stage patients were 36/38 GD2+ive. For late stage patients 66/75 had abnormal levels of CA-125 (966±527 U/1) and 65 of them were GD2+ive. The other 9/75 had normal CA-125 levels, and 8 of those 9 were GD2+ive. Hence the ELISA would have detected 8/9 late stage patients that otherwise would not be detected. Overall the advance stage patients were 73/75 GD2-positive.

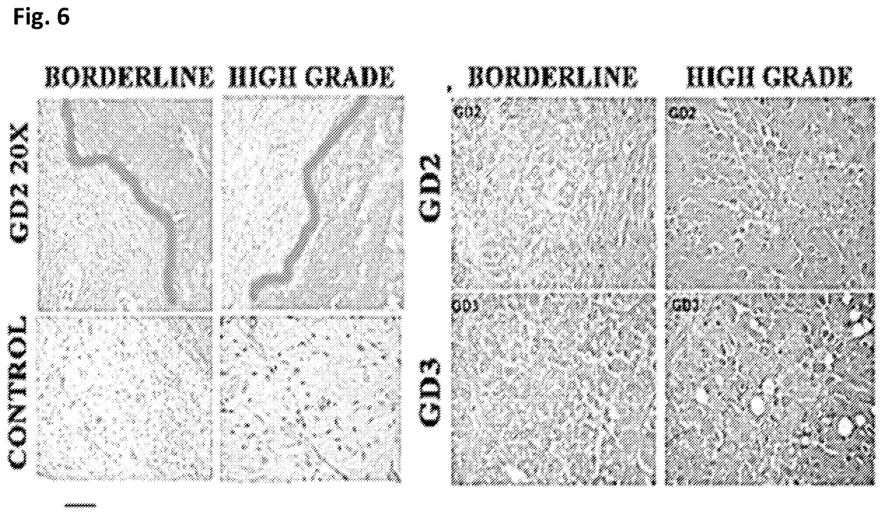

The data for early stage diagnosis is further highlighted by the relative staining intensity. There is a significant direct correlation of single staining intensity for GD3 (p=0.026) and GD2 (p=0.015) with cancer stage (FIG. 5). Such data indicate a correlation with lack of response to platinum-chemotherapies, which could help drive personalized therapeutic decisions.

These are retrospective studies, double-blinded, scored by a pathologist, under approved Ethics protocols. Tumor Microarrays (TMAs), with standard DAB staining procedures were also used. In addition, both manual and automated methods in Molecular Pathology Services (Discovery XTautomated IHC platform; Ventana, ROCHE) have been used.

These data support the diagnostic use of GD2 and GD3 markers. Current diagnostic pathology guidelines include stains for CK20, ER, PAX8, P53, and CK7 to differentiate ovarian form metastatic colorectal cancers. Adding GD2 and GD3 to the list would addresses a need for better diagnostic pathology and prediction of responses.

### Example 6: LC/MS Assay in Liquid Biopsies-Level of Gangliosides

Using nLC-ESI-MS/MS (LC/MS), liquid biopsies from 6 cancer patients (2 Melanoma, 2 Renal cancer, 2 SCLC newly diagnosed-treatment naïve) and 3 non-cancer controls (2 donors studied individually, and 1 pooled plasma from 30 donors) were analyzed (Tables 5 and 6). The LC/MS method was developed to study all tumor gangliosides at once (e.g. the Cancer Gangliosome Matrix, comprising 20 tumor gangliosides within at least 5,000 analytes).

Cancer patients showed elevated tumor gangliosides, which were specific to each cancer. Assesed by LC-MS, and compared to non-cancer samples, melanoma samples showed a significant increase in the level of GM2, GD3 and GD1b; renal cancer samples showed a significant increase in the level of GD3 and GD2; and lung cancer samples showed a significant increase in the level of GD3 and GM2. In the studies of ovarian cancer samples done specifically to evaluate GD3, 12/13 ovarian cancer samples demonstrated a significant increase in GD3 compared to 2 non-cancer control samples. Control non-cancer samples showed the level that was very low or below the threshold of detection (Table 5). Internal standards included Cholesterol (not shown) and Phosphatidylcholine (not shown) which were present at high levels in all samples. Phosphatidylserine (PS), a non-specific positive marker of general stress (cancer, inflammation, diabetes, infection, sepsis, apoptosis), was elevated in all cancer samples over control non-cancer samples.

TABLE 5
LC-MS-MS detection of gangliosides as diagnostics of cancer.

| Tumor | | Renal | Lung | Non- | Ovarian | Non- |
|---|---|---|---|---|---|---|
| Ganglioside | Melanoma | Cancer | Cancer | cancer | Cancer | cancer |
| GM2 | 1,350 | 60 | 140 | 14 | | |
| GD3 | 430 | 200 | 100 | 10 | 213 | 33 |
| GD2 | 45 | 155 | 20 | 15 | | |
| GD1b | 80 | 0 | 0 | 0 | | |
| PS | 60,000 | 26,000 | 18,000 | 4,000 | | |

Left Panel is 1 patient per case, with relative quantification. The limit of Quantifiable Detection (LOQD) was ˜1-20 units. Right Panel is absolute quantification φmol/ml). Glycolipids, measured in serum that was taken at the time of diagnosis, compared the average of thirteen ovarian cancer samples (4 early stage and 9 late stage) to the average of 2 non-cancer samples.

### Example 7: LC/MS Assay in Liquid Biopsies-Lipid Tail Length

In addition to demonstrating by LC/MS that cancer patients have elevated levels of tumor gangliosides, provided herein for the first time is a surprising and unexpected finding that the tumor gangliosides have lipid tail heterogeneity patterns that are specific to each cancer.

In melanoma, GM2 with the short chain forms (lipid tails) predominated and were increased 10-fold above normal, whereas the long chain forms increased from undetectable in normal to 90 units in cancer. For GD3, the short chain forms increased 40-fold above normal. For GD1 short chain forms increased from undetectable in normal to 70 units. Renal cancer had significant increases in GD3 and GD2 (particularly in short lipid chain forms). Lung cancer had significant increases or shifts in GD3 (particularly in long lipid chain forms) and a shift in GM2 to short lipid chain forms (Table 6). All control samples had signatures very low or below threshold, but normal gangliosides such as GM1 were detected.

As demonstrated herein for the first time, using LC/MS, the present disclosure detected and quantified the lipid variable length of gangliosides, and established the association of the lipid tail length with the cancer diagnosis. In addition, as further demonstrated herein, the LC/MS method detected other ganglioside tumor markers such as GM2 and GD1, which currently cannot be quantified in ELISA. Detection of a specific signature for many tumor gangliosides (e.g. the Cancer Gangliosome Matrix) would be of value for expanded applications and detection/diagnosis/prognosis of tumors.

TABLE 6
LC-MS-MS detection of gangliosides
as diagnostic of human cancer.

| | | | | | | | Non-cancer | |
|---|---|---|---|---|---|---|---|---|
| | Melanoma | | Renal Cancer | | Lung Cancer | | controls | |
| | Short | Long | Short | Long | Short | Long | Short | Long |
| Analyte | lipid | lipid | lipid | lipid | lipid | lipid | lipid | lipid |
| GM2 | 1260 | 90 | 0 | 60 | 20 | 120 | 140 | 0 |
| GD3 | 380 | 50 | 110 | 90 | 30 | 70 | 10 | 10 |
| GD2 | 30 | 15 | 155 | 0 | 15 | 5 | 15 | 60 |
| GD1 | 70 | 10 | 0 | 0 | 0 | 0 | 0 | 0 |

Glycolipids and lipids of serum were studied at time of diagnosis. Only relevant data compared to normal controls are shown, in relative units. For simplicity, the lipid tails are segregated as short (14-34 carbons) or long (36-48 carbons). Examples shown include a melanoma with extensive metastatic disease; a kidney cancer, and a non-small cell lung cancer. Patients were compared to the average of non-cancer donors. Example of data from ˜5,000 analytes (significant changes vs. normal in bold). Other gangliosides remain unchanged or undetectable. The currently estimated Limit of Quantifiable Detection (LOQD) is ˜40 units, or 0.010-0.24 ρmol/mL.

The LC/MS method is useful in cross-validating the ELISA, using the same liquid biopsy samples of the cohorts used for ELISA. Overall, ELISA and LC/MS on blood, and IHC of tissues provide robust data cross-validation. However, the LC/MS method has its own intrinsic value, as it measures many unique features of GD2 and GD3 signatures that the IHC and the ELISA do not measure.

### Example 8: The Level of GD2/GD3 Indicates Responsiveness to Cancer Therapy

The presence of GD2 or GD3 in the blood sample of melanoma cancer patients treated with different cancer therapies (Table 7), e.g., immune checkpoint inhibition therapy and/or chemotherapy, was detected using ELISA. The level of GD2/GD3 correlated with responsiveness to various cancer therapies as determined by PET imaging. The low level or undetectable level of GD2 or GD3 correlated with better response to treatment with various cancer therapies.

TABLE 7
Responsiveness to cancer therapy

| Case | GD2 | GD3 | Therapy | 1-year response (PET) |
|---|---|---|---|---|
| 1 | − | − | Avelumab (PDL-1) + | Almost complete (90% |
| | | | BRAF/MEK | response to the therapy) |
| 2 | +++ | + | Pembrolizumab (PD-1) | Progression-death (0% |
| | | | | response to the therapy) |
| 3 | ++ | ++ | Nivolumab (PD-1) + | Partial (60% response |
| | | | Ipilimumab (CTLA-4) | to the therapy) |
| 4 | ++ | − | Nivolumab (PD-1) + | Partial (50% response |
| | | | BRAF/MEK | to the therapy) |
| 5 | − | − | Pembrolizumab (PD-1) | Complete (100% response |
| | | | | to the therapy) |

### Example 9: Direct Binding ELISA

Native gangliosides (GD2 or GD3, Control), Tria-tri-GD2, Tria-tri-GD3, PAMAM-GD2, PAMAM-GD3, or the monomeric precursors AP-GD2 or AP-GD3 were immobilized onto polystyrene Corning Strip Well 96-well plates (10 ng/well). After bocking all wells with albumin (BSA), the wells were reacted for binding by anti-GD2 or anti-GD2 mAbs (7 nM final) or with serum from mice vaccinated with PAMAM-GD2 (1:75 dilution). Secondary antibody used was horseradish peroxidase (HRP)-conjugated anti-mouse IgG (Sigma). BG=background control.

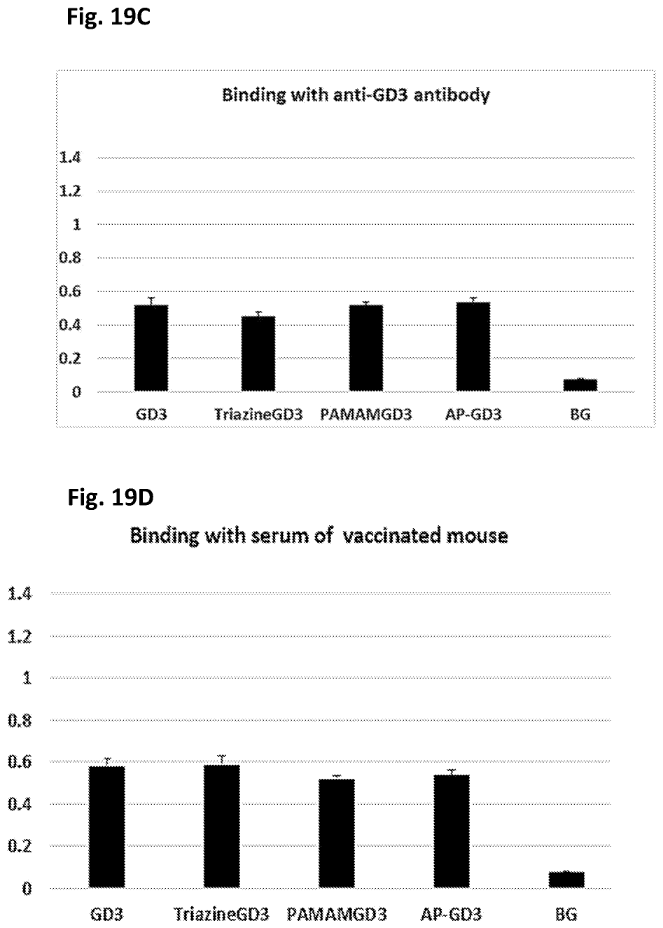

Referring to Table 8 and FIGS. 19A-19D, both Triazine-triGD2 and Triazine-triGD3 were recognized and bound by anti-GD2 or anti-GD3 mAbs, respectively. In addition, antisera raised against PAMAM-GD2 or PAMAM-GD3 cross-reacted with and bound directly to Triazine-triGD2 or Triazine-triGD3, indicating that the carbohydrate moieties are the same immunogen in PAMAM-GD2 and Triazine-triGD2 for example.

TABLE 8
Direct Binding ELISA data

| | | | | |
|---|---|---|---|---|
| OD(450 nm) | Average | StDev | SEM | Count |

GD2 versions with anti-GD2 antibody

| | | | | | | | |
|---|---|---|---|---|---|---|---|
| Control | 1.174 | 1.069 | 1.072 | 1.105 | 0.059774577 | 0.034510868 | 3 |
| TriazineGD2 | 0.506 | 0.478 | 0.39 | 0.458 | 0.060530984 | 0.03494758 | 3 |
| PAMAMGD2 | 0.541 | 0.533 | 0.485 | 0.51966667 | 0.030287511 | 0.017486503 | 3 |
| AP-GD2 | 0.483 | 0.558 | 0.568 | 0.53633333 | 0.046457866 | 0.026822462 | 3 |
| BG | 0.08 | 0.078 | 0.069 | 0.07566667 | 0.005859465 | 0.003382964 | 3 |

GD2 versions with vaccinated mouse sera

| | | | | | | | |
|---|---|---|---|---|---|---|---|
| GD2 | 1.319 | 1.23 | 1.128 | 1.22566667 | 0.095573706 | 0.055179505 | 3 |
| TriazineGD2 | 0.537 | 0.442 | 0.559 | 0.51266667 | 0.062179847 | 0.035899551 | 3 |
| PAMAMGD2 | 0.703 | 0.547 | 0.628 | 0.626 | 0.078019228 | 0.045044423 | 3 |
| AP-GD2 | 0.694 | 0.587 | 0.635 | 0.63866667 | 0.053594154 | 0.030942599 | 3 |
| BG | 0.08 | 0.078 | 0.069 | 0.07566667 | 0.005859465 | 0.003382964 | 3 |

GD3 versions with anti-GD3 antibody

| | | | | | | | |
|---|---|---|---|---|---|---|---|
| GD3 | 0.602 | 0.465 | 0.493 | 0.52 | 0.072380937 | 0.041789153 | 3 |
| TriazineGD3 | 0.44 | 0.422 | 0.502 | 0.45466667 | 0.041968242 | 0.024230376 | 3 |
| PAMAMGD3 | 0.541 | 0.533 | 0.485 | 0.51966667 | 0.030287511 | 0.017486503 | 3 |
| AP-GD3 | 0.483 | 0.558 | 0.568 | 0.53633333 | 0.046457866 | 0.026822462 | 3 |
| BG | 0.08 | 0.078 | 0.069 | 0.07566667 | 0.005859465 | 0.003382964 | 3 |

GD3 versions with vaccinated mouse sera

| | | | | | | | |
|---|---|---|---|---|---|---|---|
| GD3 | 0.646 | 0.534 | 0.555 | 0.57833333 | 0.059534304 | 0.034372146 | 3 |
| TriazineGD3 | 0.675 | 0.548 | 0.539 | 0.58733333 | 0.076054805 | 0.043910262 | 3 |
| PAMAMGD3 | 0.541 | 0.533 | 0.485 | 0.51966667 | 0.030287511 | 0.017486503 | 3 |
| AP-GD3 | 0.483 | 0.558 | 0.568 | 0.53633333 | 0.046457866 | 0.026822452 | 3 |
| BG | 0.08 | 0.078 | 0.069 | 0.07566667 | 0.005859465 | 0.003382964 | 3 |

*BG: Background

### Example 10: Competition ELISA

Native gangliosides GD2 or GD3 (Advanced Immunochemical Inc.) were immobilized onto polystyrene Corning Strip Well 96-well plates (10 ng/well) and tested for binding of anti-GD2 or anti-GD3 mAbs. Secondary antibody used was horseradish peroxidase (HRP)-conjugated anti-mouse IgG (Sigma). During the ELISA, competition was carried out by adding Triazine-tri-GD2 or Triazine-tri-GD3 as a competing ligand.

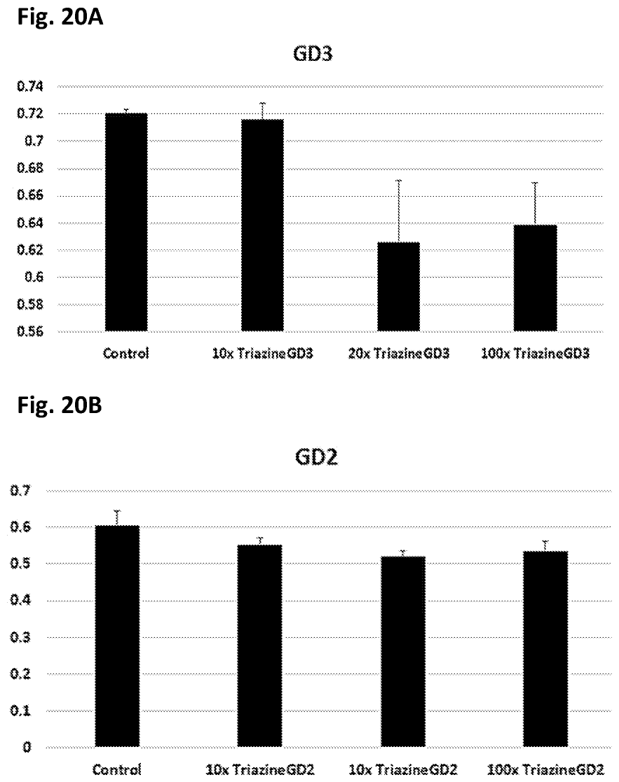

Referring to Table 9 and FIGS. 20A and 20B, both Triazine-triGD2 and Triazine-triGD3 were recognized by anti-GD2 or anti-GD3 mAbs, respectively; resulting in competition of the mAbs binding to GD2 or GD3 immobilized onto the plates.

TABLE 9

| | | | | |
|---|---|---|---|---|
| OD(450 nm) | Average | StDev | SEM | Count |

Triazine-triGD2

| | | | | | | | |
|---|---|---|---|---|---|---|---|
| Control | 0.597 | 0.68 | 0.541 | 0.606 | 0.069935685 | 0.040377386 | 3 |
| 10x TriazineGD2 | 0.573 | 0.569 | 0.519 | 0.55366667 | 0.030088758 | 0.017371752 | 3 |
| 10x TriazineGD2 | 0.541 | 0.533 | 0.485 | 0.51966667 | 0.030287511 | 0.017486503 | 3 |
| 100x TriazineGD2 | 0.483 | 0.558 | 0.568 | 0.53633333 | 0.046457866 | 0.026822462 | 3 |

Triaizne-triGD3

| | | | | | | | |
|---|---|---|---|---|---|---|---|
| Control | 0.726 | 0.719 | 0.716 | 0.72033333 | 0.005131601 | 0.002962731 | 3 |
| 10x TriazineGD3 | 0.723 | 0.732 | 0.693 | 0.716 | 0.020420578 | 0.011789826 | 3 |
| 20x TriazineGD3 | 0.703 | 0.547 | 0.628 | 0.626 | 0.078019228 | 0.045044423 | 3 |
| 100x TriazineGD3 | 0.694 | 0.587 | 0.635 | 0.63866667 | 0.053594154 | 0.030942599 | 3 |

### Example 11: Sandwich ELISA

1. Coating solution is prepared by diluting the anti-GD2/GD3 capture antibody (1:1000-1:5000 dilution) in coating buffer (10 mM phosphate).
2. 100 μL of coating antibody solution is added per well of the microtiter plate.
3. The plate is kept for overnight incubation (up to 18 hours) in refrigerated conditions (2-8° C.).
4. Next day, the solution is aspirated, and plate is washed once with 200 μL of wash buffer (Tris or PBS with 0.05% tween-20). The microtiter plate is inverted and tapped on adsorbent towel to remove any excess liquid.
5. 200 μL Blocking buffer (PBS with 0.1% BSA or 5% non-fat skimmed milk or 1% casein) per well is added and plate is incubated for 1 hour at room temperature.
6. The solution is aspirated, and the microtiter plate is inverted and tapped on adsorbent towel to remove any excess liquid.
7. Samples and standards with various amounts of GD2/GD3 are prepared in blocking buffer and added in duplicates (100 μL per well) to bind to the capture antibody on the plate, either carbohydrate or lipid moiety is bound to the antibody.
8. The plate is incubated for 2 hours at room temperature with gentle shaking (˜300-500 rpm). During this incubation, the micelles comprising gangliosides are captured by the antibody.
9. After incubation, the solution is aspirated, and the microtiter plate is inverted and tapped on adsorbent towel to remove any excess liquid.
10. The plate is washed with 200 μL of wash buffer (Tris or PBS with 0.05% tween-20) 3-4 times.
11. Following each wash, the microtiter plate is inverted and tapped on adsorbent towel to remove any excess liquid.
12. The anti-GD2/GD3 detection antibody labeled with a horse radish peroxidase (HRP) is prepared with appropriate dilution in blocking buffer.
13. 100 μL of the detection antibody is added per well and the plate is incubated for 3 hours at room temperature with gentle shaking (˜300-500 rpm).
14. After incubation, the solution is aspirated, and the microtiter plate is inverted and tapped on adsorbent towel to remove any excess liquid.
15. The plate is washed with 200 μL of wash buffer (Tris or PBS with 0.05% tween-20) 3-4 times.
16. After each wash, the microtiter plate is inverted and tapped on adsorbent towel to remove any excess liquid.
17. Working solution of streptavidin-HRP (1:5000 dilution) is prepared in blocking buffer.
18. 100 μL of the streptavidin-HRP solution is added per well and the plate is incubated for 45 minutes at room temperature with gentle shaking (˜300-500 rpm).
19. After incubation, the solution is aspirated, and the plate is washed with 200 μL of wash buffer (Tris or PBS with 0.05% tween-20) 3-4 times.
20. After each wash, the microtiter plate is inverted and tapped on adsorbent towel to remove any excess liquid.
21. 100 μL of the TMB substrate is added per well and the plate is incubated for 30 minutes at room temperature.
22. 100 μL of the stop solution is added to each well and absorbance is measured at 450 nm within 30 minutes of stop solution addition.
23. Standard curve is prepared using known amounts of GD2/GD3 and their correlation with the ODs observed. Concentrations for the samples are calculated using their observed ODs and standard curve fit.

### Example 12: Preparation of Samples Extracted from Serum for ELISA Studies-Lipid Extraction Using CHCl₃:MEOH:H₂O

To extract glycolipids from a 100 μL serum volume, the sample was centrifuged at 3000×g for 10 min and the supernatant was collected free of pellet. Then, 5 volumes of CHCl₃:MeOH:H₂O [chloroform:methanol: water, e.g., ratios ranging from 1:2:1, or 4:8:3, or 2:4:1] were added to the collected supernatant, e.g., for extraction and/or concentration of the desired analyte. The mixture was centrifuged at 3000×g for 10 minutes, and the supernatant was recovered avoiding the interphase. To the recovered supernatant, 26 μl of distilled water was added And then the extraction steps were repeated once, and the supernatant recovered after the second extraction. If organic solvents were recovered, the organic phase was evaporated (e.g., in a Speed-Vac). The extracted samples (“ELISA samples”) were used for ELISA as described herein.

### Example 13: ELISA of Lipids Extracted Using CHCl₃:MEOH:H₂O

1. The ELISA samples were prepared as above. ELISA samples or pure ganglioside (used as standard positive control) were mixed 1:2 with 95% ethanol. ELISA plates were then coated with the ELISA samples or with a standard curve of positive control native ganglioside, by drying the material added to the wells at room temperature or in a 30° C. box for ˜5 minutes. 96-well ELISA plates were exemplified herein, but other formats are possible.
2. To all wells, the freshly made PBS-BSA 0.1% blocking buffer was added and incubated for 30-45 min to block non-specific binding in the wells.
3. After removing the blocking buffer, 50 μl of primary antibody, e.g., anti-GD2 or anti-GD3 antibodies of this disclosure, was added. The concentrations of the primary mAbs ranged from 1 μg/ml to 0.005 ug/ml.
4. The primary antibody was incubated for 30-60 min.
5. The wells were washed three times with 200 μl/well of the blocking buffer.
6. After removing the blocking buffer, 50 μl of a diluted secondary antibody, which was enzyme-conjugated or fluorescently-labeled, was added and incubated for 30-60 min.
7. The wells were washed three times with 200 μl/well of PBS.
8. A standard detection system for the tag of the secondary antibody is then used. In the case of horseradish peroxidase tagged secondary antibody, 50 μl of TMB reagent was added per well (TMB-ELISA Thermo Scientific [Catalog #34028].).
9. The plate was incubated for ˜3 to 5 min or until the wells containing the positive control (for generating a standard curve) reached a light blue color.
10. The reaction was stopped by adding 50 μl of 0.5N H2SO4.
11. Reaction was read with spectrophotometer at absorbance of 450 nm.

TABLE 10
Exemplary map to immobilizing samples

| | 1 | 2 | 3 | 4 | 5 | 6 | 7 | 8 | 9 | 10 | 11 | 12 |
|---|---|---|---|---|---|---|---|---|---|---|---|---|
| A | S1 | S2 | S3 | S4 | S5 | S6 | S7 | S8 | S9 | S10 | S11 | S12 |
| B | S1 | S2 | S3 | S4 | S5 | S6 | S7 | S8 | S9 | S10 | S11 | S12 |
| C | Bkg | Bkg | Bkg | Bkg | Bkg | Bkg | Bkg | Bkg | Bkg | Bkg | Bkg | Bkg |
| D | Bkg | Bkg | Bkg | Bkg | Bkg | Bkg | Bkg | Bkg | Bkg | Bkg | Bkg | Bkg |
| E | S13 | S14 | S15 | S16 | S17 | S18 | S19 | S20 | −Ctl | +Ctl | N1Ctl | N2Ctl |
| F | S13 | S14 | S15 | S16 | S17 | S18 | S19 | S20 | −Ctl | +Ctl | N1Ctl | N2Ctl |
| G | Bkg | Bkg | Bkg | Bkg | Bkg | Bkg | Bkg | Bkg | Bkg | Bkg | Bkg | Bkg |
| H | Bkg | Bkg | Bkg | Bkg | Bkg | Bkg | Bkg | Bkg | Bkg | Bkg | Bkg | Bkg |

Codes for Table 10:
S (1-20): samples from the lipid extraction
−Ctl: negative control
+Ctl: positive control
N (1-2) Ctl: negative control samples from normal patients
Bkg: background using only secondary antibody

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and amino acid sequences which reference an accession number correlating to an entry in a public database, such as those maintained by The Institute for Genomic Research (TIGR) on the World Wide Web at tigr. org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the present invention described herein. Such equivalents are intended to be encompassed by the following claims.

### REFERENCES

1. Howlader N. et al. Lifetime Risk (Percent) of Being Diagnosed with Cancer by Site and Race/Ethnicity; Males, 18 SEER Areas, 2012-2014SEER Cancer Statistics Review, 1975-2014, National Cancer Institute. Bethesda, MD, https://seer.cancer.gov/csr/1975_2014/, based on November 2016 SEER data submission, posted to the SEER web site, April 2017.
2. Cancer Stat Facts: Ovarian Cancer. Accessed 15 Jan. 2020. <World Wide Web at seer.cancer.gov/statfacts/html/ovary.html>
3. American Cancer Society. Cancer Facts and Figures . 2018. Atlanta, GA: American Cancer Society; 2018.
4. van Nagell J R Jr, Hoff J T. Transvaginal ultrasonography in ovarian cancer screening: current perspectives. Int J Womens Health. 2013; 6:25-33. Published 2013 Dec. 20.
5. ACOG Statement on FDA Safety Communication on Ovarian Cancer Screening Tests. 8 Sep. 2016. <World Wide Web at acog.org/About-ACOG/News-Room/Statements/2016/ACOG-Statement-on-FDA-Safety-Communication-on-Ovarian-Cancer-Screening-Tests?IsMobileSet=false>
6. Ovarian Cancer Canada 2019. 15 Jan. 2020. <World Wide Web at ovariancanada.org/About-Ovarian-Cancer/Detection>Esselen KM, Cronin AM, Bixel K, et al. Use of CA-125 Tests and Computed Tomographic Scans for Surveillance in Ovarian Cancer. JAMA Oncol. 2016; 2 (11): 1427-1433.
7. Pepin K. et al. CA 125 and Epithelial Ovarian Cancer: Role in Screening, Diagnosis, and Surveillance.
8. Dochez V, Caillon H, Vaucel E, Dimet J, Winer N, Ducarme G. Biomarkers and algorithms for diagnosis of ovarian cancer: CA125, HE4, RMI and ROMA, a review. J Ovarian Res. 2019; 12 (1): 28. Published 2019 Mar. 27.
9. Risk of Malignancy Index (RMI) for Ovarian Cancer. 15 Jan. 2020. <World Wide Web at mdcalc.com/risk-malignancy-index-rmi-ovarian-cancer>
10. Ovarian Cancer Testing: HE4, Ovarian Cancer Monitoring. 15 Jan. 2020. <World Wide Web at questdiagnostics.com/home/patients/health-test-info/cancer/ovarian-cancer/he4-monitoring/>Monk B. Monitoring and Managing Patients With Ovarian Cancer. 28 Feb. 2018. <World Wide Web at targetedonc.com/case-based-peer-perspectives/gynecologic-cancer/monk-recurrentovarian-cancer/monitoring-and-managing-patients-with-ovarian-cancer>
11. Herzog T J. Patient education: Treatment of ovarian cancer (Beyond the Basics). 28 Jan. 2020. <World Wide Web at uptodate.com/contents/treatment-of-ovarian-cancer-beyond-the-basics>
12. Women & Infants Hospital. “FDA clears biomarker test for ovarian cancer.” ScienceDaily, 6 Sep. 2011. <World Wide Web at sciencedaily.com/releases/2011/09/110906144034.htm>.
13. k081754-510 (k) SUBSTANTIAL EQUIVALENCE DETERMINATION DECISION SUMMARY. 15 Jan. 2020. <World Wide Web at accessdata.fda.gov/cdrh_docs/reviews/K081754.pdf>
14. Moore R G, McMeekin D S, Brown A K, et al. A novel multiple marker bioassay utilizing HE4 and CA125 for the prediction of ovarian cancer in patients with a pelvic mass. Gynecol Oncol. 2009; 112:40-6.
15. Dunton, G. et al. (2019) Multivariate Index Assay Outperforms CA125 in Detection of Ovarian Malignancy in African American Women. Biomarkers in Cancer 11:1-4
16. Esselen K M, Cronin A M, Bixel K, et al. Use of CA-125 Tests and Computed Tomographic Scans for Surveillance in Ovarian Cancer. JAMA Oncol. 2016; 2 (11): 1427-1433. 1842 Vermillion Reports Fourth Quarter and Year-End 2018 Financial Results. 15 Jan. 2020. <World Wide Web at globenewswire.com/news release/2019/03/28/1783632/0/en/Vermillion-Reports-Fourth-Quarter-and-Year-End-2018-Financial-Results.html>
17. Nolen B M, Lokshin A E. Biomarker testing for ovarian cancer: clinical utility of multiplex assays. Mol Diagn Ther. 2013; 17 (3): 139-146. doi: 10.1007/s40291-013-0027-6
18. Bercow A S, Chen L, Chatterjee S, et al. Cost of Care for the Initial Management of Ovarian Cancer. Obstet Gynecol. 2017;130 (6): 1269-1275.
19. How much does treating ovarian cancer cost? 15 Jan. 2020. <World Wide Web at herwaroncancer.com/common-types-of-cancer/ovarian-cancer/treatment-cost/>
20. Jacobs et al., I et al. (2015) Ovarian cancer screening and mortality in the UK Collaborative Trial of Ovarian Cancer Screening (UKTOCS): a randomised controlled trial. The Lancet. 10222:945-956
21. Buys, S et al., (2011) Effect of screening on ovarian cancer mortality: the Prostate, Lung, Colorectal and Ovarian (PLCO) Cancer Screening Randomized Controlled Trial. JAMA. (22): 2295-303
22. World Wide Web at nccn.org/professionals/physician_gls/default.aspx
23. Cannistra, S. A. Cancer of the ovary. N Engl J Med 351, 2519-2529, (2004).
24. Daly, M. B. & Ozols, R. F. Symptoms of ovarian cancer-—where to set the bar? JAMA 291, 2755-2756, (2004).
25. Vaughan, S. et al. Rethinking ovarian cancer: recommendations for improving outcomes. Nat Rev Cancer 11, 719-725, doi: 10.1038/nrc3144 (2011).
26. Averette, H. E. et al. National survey of ovarian carcinoma. I. A patient care evaluation study of the American College of Surgeons. Cancer 71, 1629-1638 (1993).
27. Felder, M. et al. MUC16 (CA125): tumor biomarker to cancer therapy, a work in progress. Mol Cancer 13, 129, doi: 10.1186/1476-4598-13-129 (2014).
28. Santin, A. D. et al. Increased levels of gangliosides in the plasma and ascitic fluid of patients with advanced ovarian cancer. BJOG 111, 613-618, (2004).
29. Shenoy, G. N. et al. Sialic Acid-Dependent Inhibition of T Cells by Exosomal Ganglioside GD3 in Ovarian Tumor Microenvironments. J Immunol, (2018).
30. Tiper, I. V. et al. VEGF Potentiates GD3-Mediated Immunosuppression by Human Ovarian Cancer Cells. Clin Cancer Res 22, 4249-4258, (2016).
31. Cancer Genome Atlas Research, N. Integrated genomic analyses of ovarian carcinoma. Nature 474, 609-615, (2011).
32. Gagnon, M. & Saragovi, H. U. Gangliosides: therapeutic agents or therapeutic targets? Expert Opinion on Therapeutic Patents 12, 1215-1223, (2002).
33. Daniotti, J. L., Lardone, R. D. & Vilcaes, A. A. Dysregulated Expression of Glycolipids in Tumor Cells: From Negative Modulator of Anti-tumor Immunity to Promising Targets for Developing Therapeutic Agents. Front Oncol 5, 300, (2016).
34. Zhang, X. & Kiechle, F. L. Review: Glycosphingolipids in health and disease. Ann Clin Lab Sci 34, 3-13 (2004).
35. Regina Todeschini, A. & Hakomori, S. I. Functional role of glycosphingolipids and gangliosides in control of cell adhesion, motility, and growth, through glycosynaptic microdomains. Biochimica et Biophysica Acta 1780, 421-433, (2008).
36. Navid, F., Santana, V. M. & Barfield, R. C. Anti-GD2 antibody therapy for GD2− expressing tumors. Current Cancer Drug Dargets 10, 200-209 (2010).
37. Cheever, M. A. et al. The prioritization of cancer antigens: a national cancer institute pilot project for the acceleration of translational research. Clin Cancer Res 15, 5323-5337, (2009).
38. Yang, R. K. & Sondel, P. M. Anti-GD2 Strategy in the Treatment of Neuroblastoma. Drugs Future 35, 665 (2010).
39. Chiantia, S., Kahya, N. & Schwille, P. Raft domain reorganization driven by short- and longchain ceramide: a combined AFM and FCS study. Langmuir 23, 7659-7665, (2007).
40. Grey, M. et al. Acceleration of alpha-synuclein aggregation by exosomes. J Biol Chem 290, 2969-2982, (2015).
41. Yu, R. K., Tsai, Y. T., Ariga, T. & Yanagisawa, M. Structures, biosynthesis, and functions of gangliosides-—an overview. J Oleo Sci 60, 537-544 (2011).
42. Chen, Y. X. et al. Effect of tumor gangliosides on tyrosine phosphorylation of p125FAK in platelet adhesion to collagen. Oncol Rep 29, 343-348, (2013).
43. Shibuya, H. et al. Enhancement of malignant properties of human osteosarcoma cells with disialyl gangliosides GD2/GD3. Cancer Sci 103, 1656-1664, (2012).
44. Furukawa, K., Hamamura, K., Ohkawa, Y., Ohmi, Y. & Furukawa, K. Disialyl gangliosides enhance tumor phenotypes with differential modalities. Glycoconj J 29, 579-584, (2012).
45. Hamamura, K. et al. Functional activation of Src family kinase yes protein is essential for the enhanced malignant properties of human melanoma cells expressing ganglioside GD3. J Biol Chem 286, 18526-18537 (2011).
46. Mitsuzuka, K., Handa, K., Satoh, M., Arai, Y. & Hakomori, S. A specific microdomain (“glycosynapse 3”) controls phenotypic conversion and reversion of bladder cancer cells through GM3-mediated interaction of alpha3betal integrin with CD9. J. Biol. Chem. 280, 35545-35553, (2005).
47. Yoshida, S. et al. Ganglioside G (D2) in small cell lung cancer cell lines: enhancement of cell proliferation and mediation of apoptosis. Cancer Research 61, 4244-4252 (2001).
48. Daniotti, J. L., Crespo, P. M. & Yamashita, T. In vivo modulation of epidermal growth factor receptor phosphorylation in mice expressing different gangliosides. Journal of Cellular Biochemistry 99, 1442-1451 (2006).
49. Ohkawa, Y. et al. Ganglioside GD3 Enhances Invasiveness of Gliomas by Forming a Complex with Platelet-derived Growth Factor Receptor alpha and Yes Kinase. J Biol Chem 290, 16043-16058, (2015).
50. Rampersaud, A. A., Oblinger, J. L., Ponnappan, R. K., Burry, R. W. & Yates, A. J. Gangliosides and growth factor receptor regulation. Biochem Soc Trans 27, 415-422 (1999).
51. Tong, W. et al. Small-molecule ligands of GD2 ganglioside, designed from NMR studies, exhibit induced-fit binding and bioactivity. Chem Biol 17, 183-194 (2010).
52. Tong, W., Maira, M., Gagnon, M. & Saragovi, H. U. Ligands Binding to Cell Surface Ganglioside GD2 Cause Src-Dependent Activation of N-Methyl-D-Aspartate Receptor Signaling and Changes in Cellular Morphology. PLOS One 10, e0134255, (2015).
53. Sorice, M. et al. Role of GM3-enriched microdomains in signal transduction regulation in T lymphocytes. Glycoconjugate Journal 20, 63-70, (2004).
54. Kasahara, K., Watanabe, Y., Yamamoto, T. & Sanai, Y. Association of Src family tyrosine kinase Lyn with ganglioside GD3 in rat brain. Possible regulation of Lyn by glycosphingolipid in caveolae-like domains. J Biol Chem 272, 29947-29953 (1997).
55. Yates, A. J. & Rampersaud, A. Sphingolipids as receptor modulators. An overview. Ann NY Acad Sci 845, 57-71 (1998).
56. Liu, Y., McCarthy, J. & Ladisch, S. Membrane ganglioside enrichment lowers the threshold for vascular endothelial cell angiogenic signaling. Cancer Research 66, 10408-10414, (2006).
57. Ziche, M., Morbidelli, L., Alessandri, G. & Gullino, P. M. Angiogenesis can be stimulated or repressed in vivo by a change in GM3: GD3 ganglioside ratio. Laboratory Investigation 67, 711-715 (1992).
58. Iwabuchi, K. et al. Reconstitution of membranes simulating “glycosignaling domain” and their susceptibility to lyso-GM3. J. Biol. Chem. 275, 15174-15181 (2000).
59. Todeschini, A. R., Dos Santos, J. N., Handa, K. & Hakomori, S. I. Ganglioside GM2-tetraspanin CD82 complex inhibits met and its cross-talk with integrins, providing a basis for control of cell motility through glycosynapse. J. Biol. Chem. 282, 8123-8133, (2007).
60. Nakashima, H. et al. Overexpression of caveolin-1 in a human melanoma cell line results in dispersion of ganglioside GD3 from lipid rafts and alteration of leading edges, leading to attenuation of malignant properties. Cancer Science 98, 512-520 (2007).
61. Aixinjueluo, W. et al. Mechanisms for the apoptosis of small cell lung cancer cells induced by anti-GD2 monoclonal antibodies: roles of anoikis. J. Biol. Chem. 280, 29828-29836 (2005).
62. Yoshida, S., Kawaguchi, H., Sato, S., Ueda, R. & Furukawa, K. An anti-GD2 monoclonal antibody enhances apoptotic effects of anti-cancer drugs against small cell lung cancer cells via JNK (c-Jun terminal kinase) activation. Japanese Journal of Cancer Research 93, 816-824 (2002).
63. Furukawa, K., Hamamura, K., Aixinjueluo, W. & Furukawa, K. Biosignals modulated by tumorassociated carbohydrate antigens: novel targets for cancer therapy. Annals of the New York Academy of Sciences 1086, 185-198 (2006).
64. Caldwell, S. et al. Mechanisms of ganglioside inhibition of APC function. Journal of Immunology 171, 1676-1683 (2003).
65. Shurin, G. V. et al. Neuroblastoma-derived gangliosides inhibit dendritic cell generation and function. Cancer Research 61, 363-369 (2001).
66. Grayson, G. & Ladisch, S. Immunosuppression by human gangliosides. II. Carbohydrate structure and inhibition of human NK activity. Cellular Immunology 139, 18-29 (1992).
67. Dube, D. H. & Bertozzi, C. R. Glycans in cancer and inflammation—potential for therapeutics and diagnostics. Nat Rev Drug Discov 4, 477-488 (2005).
68. Rodriguez, E., Schetters, S. T. T. & van Kooyk, Y. The tumour glyco-code as a novel immune checkpoint for immunotherapy. Nat Rev Immunol 18, 204-211, (2018).
69. Astronomo, R. D. & Burton, D. R. Carbohydrate vaccines: developing sweet solutions to sticky situations? Nat Rev Drug Discov 9, 308-324 (2010).
70. Webb, T. J. et al. Molecular identification of GD3 as a suppressor of the innate immune response in ovarian cancer. Cancer Res 72, 3744-3752, (2012).
71. Heimburg-Molinaro, J. et al. Cancer vaccines and carbohydrate epitopes. Vaccine 29, 8802-8826, (2011).
72. Doronin, I I et al. Ganglioside GD2 in reception and transduction of cell death signal in tumor cells. BMC Cancer 14, 295, doi: 10.1186/1471-2407-14-295 (2014).
73. Kramer, G., Suss, W., Kohler, H., Schneider, B. & Hopf, H. C. [Ganglioside therapy in uremic polyneuropathy]. Med Klin (Munich) 83, 7-11, 40 (1988).
74. Brignole, C. et al. Immune cell-mediated antitumor activities of GD2-targeted liposomal c-myb antisense oligonucleotides containing CpG motifs. Journal of the National Cancer Institute 96, 1171-1180, (2004).
75. Zhang, S. et al. Selection of tumor antigens as targets for immune attack using immunohistochemistry: I. Focus on gangliosides. International Journal of Cancer 73, 42-49 (1997).
76. Poon, V. I. et al. Ganglioside GD2 expression is maintained upon recurrence in patients with osteosarcoma. Clin Sarcoma Res 5, 4, doi: 10.1186/s13569-014-0020-9 (2015).
77. Kong, Y., Li, R. & Ladisch, S. Natural forms of shed tumor gangliosides. Biochim Biophys Acta 1394, 43-56 (1998).
78. Ladisch, S., Li, R. & Olson, E. Ceramide structure predicts tumor ganglioside immunosuppressive activity. Proc Natl Acad Sci USA 91, 1974-1978 (1994).
79. Irani, D. N., Lin, K. I. & Griffin, D. E. Brain-derived gangliosides regulate the cytokine production and proliferation of activated T cells. J Immunol 157, 4333-4340 (1996).
80. Wu, A. M. et al. Glycomic mapping of pseudomucinous human ovarian cyst glycoproteins: identification of Lewis and sialyl Lewis glycotopes. Proteomics 7, 3699-3717, (2007).
81. Ladisch, S. & Wu, Z. L. Detection of a tumour-associated ganglioside in plasma of patients with neuroblastoma. Lancet 1, 136-138 (1985).
82. Bernhard, H., Meyer zum Buschenfelde, K. H. & Dippold, W. G. Ganglioside GD3 shedding by human malignant melanoma cells. Int J Cancer 44, 155-160 (1989).
83. Merritt, W. D., Der-Minassian, V. & Reaman, G. H. Increased GD3 ganglioside in plasma of children with T-cell acute lymphoblastic leukemia. Leukemia 8, 816-822 (1994).
84. Valentino, L. et al. Shed tumor gangliosides and progression of human neuroblastoma. Blood 75, 1564-1567 (1990).
85. Tai, T. et al. Immunogenicity of melanoma-associated gangliosides in cancer patients. International Journal of Cancer 35, 607-612 (1985).
86. Tsuchida, T., Ravindranath, M. H., Saxton, R. E. & Irie, R. F. Gangliosides of human melanoma: altered expression in vivo and in vitro. Cancer Res 47, 1278-1281 (1987).
87. Tsuchida, T., Saxton, R. E., Morton, D. L. & Irie, R. F. Gangliosides of human melanoma. Journal of the National Cancer Institute 78, 45-54 (1987).
88. Tsuchida, T., Saxton, R. E., Morton, D. L. & Irie, R. F. Gangliosides of human melanoma. J Natl Cancer Inst 78, 45-54, doi: 10.1093/jnci/78.1.45 (1987).
89. Tsuchida, Y., Fukui, M., Sakaguchi, H. & Ishiguro, T. Analysis of lectin affinity immunoelectrophoretic profiles of serum alpha-fetoprotein from patients with yolk sac tumors and carcinomas of the gastrointestinal tract: correlations with molecular structures. Tumour Biol 10, 289-296, doi: 10.1159/000217628 (1989).
90. Cheresh, D. A., Pierschbacher, M. D., Herzig, M. A. & Mujoo, K. Disialogangliosides GD2 and GD3 are involved in the attachment of human melanoma and neuroblastoma cells to extracellular matrix proteins. J. Cell Biol. 102, 688-696, doi: 10.1083/jcb.102.3.688 (1986).
91. Pukel, C. S. et al. GD3, a prominent ganglioside of human melanoma. Detection and characterisation by mouse monoclonal antibody. J Exp Med 155, 1133-1147 (1982).
92. Cheung, N.-K. V. et al. Monoclonal Antibodies to a Glycolipid Antigen on Human Neuroblastoma Cells. Cancer Research 45, 2642-2649 (1985).
93. Rabu, C., McIntosh, R., Jurasova, Z. & Durrant, L. Glycans as targets for therapeutic antitumor antibodies. Future Oncol 8, 943-960, doi: 10.2217/fon. 12.88 (2012).
94. Rashidijahanabad, Z. & Huang, X. Recent advances in tumor associated carbohydrate antigen based chimeric antigen receptor T cells and bispecific antibodies for anti-cancer immunotherapy. Semin Immunol, 101390, (2020).
95. Cheung, N. K. et al. Single-chain Fv-streptavidin substantially improved therapeutic index in multistep targeting directed at disialoganglioside GD2. Journal of Nuclear Medicine 45, 867-877 (2004).
96. Daniotti, J. L., Vilcaes, A. A., Torres Demichelis, V., Ruggiero, F. M. & Rodriguez-Walker, M. Glycosylation of Glycolipids in Cancer: Basis for Development of Novel Therapeutic Approaches. Front Oncol 3, 306, (2013).
97. Tong, W. et al. Vaccination with Tumor-Ganglioside Glycomimetics Activates a Selective Immunity that Affords Cancer Therapy. Cell Chem Biol 26, 1013-1026 e1014, (2019).
98. Moffett, S. et al. Preparation and characterization of new anti-PSMA monoclonal antibodies with potential clinical use. Hybridoma 26, 363-372, (2007).
99. Bai, Y. et al. An agonistic TrkB mAb causes sustained TrkB activation, delays RGC death, and protects the retinal structure in optic nerve axotomy and in glaucoma. Invest Ophthalmol Vis Sci 51, 4722-4731, (2010).
100. Saragovi, H. U. et al. A TrkA-selective, fast internalizing nerve growth factor-antibody complex induces trophic but not neuritogenic signals. J Biol Chem 273, 34933-34940, (1998).
101. Saragovi, H. U. et al. Functional and physical association of a cell surface phospholipid and interleukin-2 receptor p55 (alpha) subunits. Biochim Biophys Acta 1414, 51-64 (1998).
102. Cohen, J. D. et al. Detection and localization of surgically resectable cancers with a multi-analyte blood test. Science 359, 926-930, (2018).
103. Killock, D. Diagnosis: CancerSEEK and destroy-a blood test for early cancer detection. Nat Rev Clin Oncol 15, 133, (2018).
104. Aravanis, A. M., Lee, M. & Klausner, R. D. Next-Generation Sequencing of Circulating Tumor DNA for Early Cancer Detection. Cell 168, 571-574, (2017). 105. Lin, J. et al. Exosomes: novel biomarkers for clinical diagnosis. ScientificWorldJournal (2015).

1. A composition comprising a ganglioside having the structure:
(A)x-[(P)y-(L)z]-(M)b;
wherein A is a ganglioside, or any portion thereof; x is an integer from 1 to 32; P is a heteroaryl; y is 1; L is a linker; z is an integer from 0 to 8; M is a core; and b is 0 or 1;
wherein P is optionally substituted with 1, 2, 3, 4, or 5 substituents independently selected from (1) hydrogen; (2) C₁₋₇ acyl; (3) C₁₋₂₀ alkyl; (4)amino; (5) C₃₋₁₀ aryl; (6) hydroxy; (7) nitro; (8) C₁₋₂₀ alkyl-amino; and (9)-(CH₂)_{q}CONR^{B}, where q is an integer from 0 to 4 and where R^{B} is selected from the group consisting of (a) hydrogen, (b) C₁₋₆ alkyl, (c) C₃₋₁₀ aryl, and (d) C₁₋₆ alk-C₆-10 aryl.

2-6. (canceled)

7. The composition of claim 1, wherein the structure is selected from: 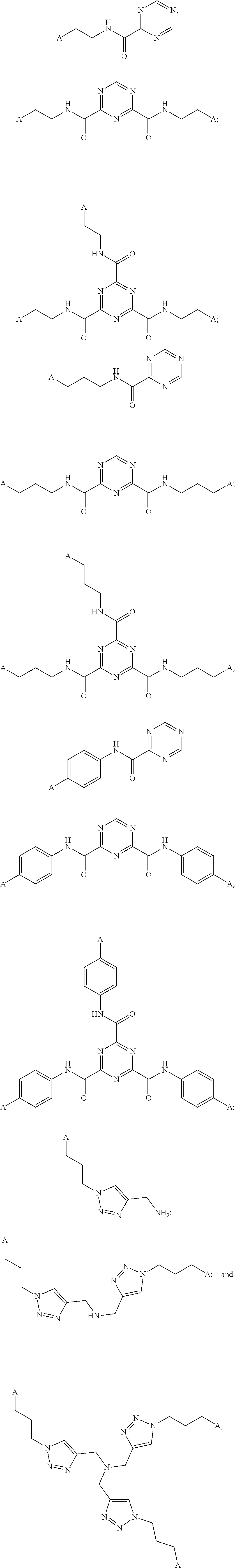
wherein A is a ganglioside.

8-10. (canceled)

11. A method of inducing an immune response against a ganglioside in a subject, comprising administering to the subject the composition of claim 1.

12. A method of treating a subject in need thereof, the method comprising administering to the subject the composition of claim 1.

13. A method of detecting the presence or level of a ganglioside in a sample, comprising detecting the ganglioside using a competitive ELISA assay, wherein the ganglioside of claim 1 is a reference antigen.

14. A method of producing an antibody in a mammal, comprising:
(a) immunizing the mammal with the composition of claim 1, optionally further comprising an adjuvant; and
(b) isolating an antibody that binds to the ganglioside from the mammal, a cell from the mammal, or a hybridoma made using a cell from the mammal.

15. (canceled)

16. A monoclonal antibody, or antigen-binding fragment thereof, wherein the monoclonal antibody specifically binds to the carbohydrate portion of a ganglioside.

17-19. (canceled)

20. The monoclonal antibody or antigen-binding fragment thereof of claim 16, comprising six CDR amino acid sequences selected from:
a) SEQ ID NOs: 2, 4, 6, 8, 10, and 12 (clone 4), or a variant sequence thereof which differs by only one or two amino acids, or which has at least or about 85% sequence identity;
b) SEQ ID NOs: 14, 16, 18, 20, 22, and 24 (clone 6), or a variant sequence thereof which differs by only one or two amino acids, or which has at least or about 85% sequence identity;
c) SEQ ID NOs: 26,28, 30, 32, 34, and 36 (clone 7), or a variant sequence thereof which differs by only one or two amino acids, or which has at least or about 85% sequence identity;
d) SEQ ID NOs: 38, 40, 42, 44, 46, and 48 (clone 8), or a variant sequence thereof which differs by only one or two amino acids, or which has at least or about 85% sequence identity;
e) SEQ ID NOs: 50, 52, 54, 56, 58, and 60 (clone 9), or a variant sequence thereof which differs by only one or two amino acids, or which has at least or about 85% sequence identity;
f) SEQ ID NOs: 62, 64, 66, 68, 70, and 72 (clone 10), or a variant sequence thereof which differs by only one or two amino acids, or which has at least or about 85% sequence identity;
g) SEQ ID NOs: 74, 76, 78, 80, 82, and 84 (clone 13), or a variant sequence thereof which differs by only one or two amino acids, or which has at least or about 85% sequence identity;
h) SEQ ID NOs: 86, 88, 90, 92, 94, and 96 (clone 14), or a variant sequence thereof which differs by only one or two amino acids, or which has at least or about 85% sequence identity;
i) SEQ ID NOs: 98, 100, 102, 104, 106, and 108 (clone 15), or a variant sequence thereof which differs by only one or two amino acids, or which has at least or about 85% sequence identity;
j) SEQ ID NOs: 110, 112, 114, 116, 118, and 120 (clone 17), or a variant sequence thereof which differs by only one or two amino acids, or which has at least or about 85% sequence identity;
k) SEQ ID NOs: 122, 124, 126, 128, 130, and 132 (clone 18), or a variant sequence thereof which differs by only one or two amino acids, or which has at least or about 85% sequence identity; and
l) SEQ ID NOs: 134, 136, 138, 140, 142, and 144 (clone 19), or a variant sequence thereof which differs by only one or two amino acids, or which has at least or about 85% sequence identity.

21. The monoclonal antibody or antigen-binding fragment thereof of claim 16 [or 17], comprising the VH and VL amino acid sequences selected from:
a) SEQ ID NOs: 146 and 148, or a variant sequence thereof which differs by only one or two amino acids, or which has at least or about 85% sequence identity;
b) SEQ ID NOs: 150 and 152, or a variant sequence thereof which differs by only one or two amino acids, or which has at least or about 85% sequence identity;
c) SEQ ID NOs: 154 and 156, or a variant sequence thereof which differs by only one or two amino acids, or which has at least or about 85% sequence identity;
d) SEQ ID NOs: 158 and 160, or a variant sequence thereof which differs by only one or two amino acids, or which has at least or about 85% sequence identity;
e) SEQ ID NOs: 162 and 164, or a variant sequence thereof which differs by only one or two amino acids, or which has at least or about 85% sequence identity;
f) SEQ ID NOs: 166 and 168, or a variant sequence thereof which differs by only one or two amino acids, or which has at least or about 85% sequence identity;
g) SEQ ID NOs: 170 and 172, or a variant sequence thereof which differs by only one or two amino acids, or which has at least or about 85% sequence identity;
h) SEQ ID NOs: 174 and 176, or a variant sequence thereof which differs by only one or two amino acids, or which has at least or about 85% sequence identity;
i) SEQ ID NOs: 178 and 180, or a variant sequence thereof which differs by only one or two amino acids, or which has at least or about 85% sequence identity;
j) SEQ ID NOs: 182 and 184, or a variant sequence thereof which differs by only one or two amino acids, or which has at least or about 85% sequence identity;
k) SEQ ID NOs: 186 and 188, or a variant sequence thereof which differs by only one or two amino acids, or which has at least or about 85% sequence identity; and
l) SEQ ID NOs: 190 and 192, or a variant sequence thereof which differs by only one or two amino acids, or which has at least or about 85% sequence identity.

22-24. (canceled)

25. An isolated nucleic acid molecule that encodes:
a) a polypeptide comprising an amino acid sequence listed in Table 1 and/or Table 2;
b) a polypeptide comprising an amino acid sequence with at least or about 85% identity to an amino acid sequence listed in Table 1 and/or Table 2; and/or
c) the monoclonal antibody or antigen-binding fragment thereof, of claim 16.

26. A vector comprising the isolated nucleic acid of claim 25.

27. A host cell which (a) comprises the isolated nucleic acid encoding the antibody or antige-binding fragment thereof of claim 16, (b) comprises the vector comprising the nucleic acid of (a), and/or (c) expresses the antibody, or antigen-binding fragment thereof, of claim 16.

28. A method of producing at least one monoclonal antibody or antigen-binding fragment thereof, according to claim 16, wherein the method comprises the steps of: (i) culturing a host cell comprising a nucleic acid comprising a sequence encoding at least one monoclonal antibody, or antigen-binding fragment thereof, according to claim 16 under conditions suitable to allow expression of said monoclonal antibody or antigen-binding fragment thereof; and (ii) recovering the expressed monoclonal antibody or antigen-binding fragment thereof.

29. A device or kit comprising at least one monoclonal antibody or antigen-binding fragment thereof, according to claim 16.

30-33. (canceled)

34. A method of purifying a ganglioside from a sample or a method of extracting lipids from a sample, the method comprising:
(a) obtaining the sample;
(b) adding to the sample about 2 to 5-fold volume of an organic solvent comprising CHCl₃: methanol: water at a ratio selected from (i) about 1:2:1, (ii) about 4:8:3, or (iii) about 2:4:1;
(c) shaking the combined mixture of (b); and
(d) separating the sample from the organic solvent, thereby extracting lipids from the sample and/or purifying a ganglioside.

35. (canceled)

36. A method of detecting the presence or level of at least one ganglioside (e.g., GD2 and/or GD3), comprising detecting said ganglioside in a sample using at least one monoclonal antibody or antigen-binding fragment thereof according to claim 16, optionally wherein the sample is from a subject afflicted with a cancer or a cancer-free subject.

37-44. (canceled)

45. A method of detecting the presence, level, or lipid length of at least one ganglioside, the method comprising detecting said ganglioside in a sample using mass spectrometry (e.g., LC/MS or LC/MS/MS), optionally wherein the sample is from a subject afflicted with a cancer or a cancer-free subject.

46. (canceled)

47. A method of diagnosing a cancer in a subject or a method of identifying a subject having a cancer, the method comprising:
a) determining the level of at least one ganglioside in a subject sample using at least one monoclonal antibody, or antigen-binding fragment thereof, optionally wherein the at least one monoclonal antibody, or antigen-binding fragment thereof is according to claim 16; and
b) comparing said level of the at least one ganglioside to that in a control sample,
wherein a significantly higher level of the at least one ganglioside in the subject sample as compared to the level in the control sample indicates that the subject has a cancer.

48. (canceled)

49. A method of determining a grade of a cancer, the method comprising:
a) determining the level of at least one ganglioside in a subject sample using at least one monoclonal antibody, or antigen-binding fragment thereof, optionally wherein the at least one monoclonal antibody, or antigen-binding fragment thereof is according to claim 16; and
b) comparing said level of the at least one ganglioside to that in a control sample,
wherein at least 100% and no more than 200% increase in the level of the at least one ganglioside in the subject sample as compared to the level in the control sample indicates that the subject has a low grade cancer; and/or
wherein at least 200% increase in the level of the at least one ganglioside in the subject sample as compared to the level in the control sample indicates that the subject has a high grade cancer.

50. A method of determining a tumor burden of a cancer, the method comprising:
a) determining the level of at least one ganglioside in a subject sample using at least one monoclonal antibody, or antigen-binding fragment thereof, optionally wherein the at least one monoclonal antibody, or antigen-binding fragment thereof is according claim 16; and
b) comparing said level of the at least one ganglioside to that in a control sample,
wherein at least 100% and no more than 200% increase in the level of the at least one ganglioside in the subject sample as compared to the level in the control sample indicates that the subject has a low tumor burden; and/or
wherein at least 200% increase in the level of the at least one ganglioside in the subject sample as compared to the level in the control sample indicates that the subject has a high tumor burden.

51. A method of detecting a recurrence of a cancer in a subject, the method comprising:
a) obtaining or providing a sample from the subject whose cancer has regressed after receiving cancer treatment;
b) determining the level of at least one ganglioside in the subject sample using at least one monoclonal antibody, or antigen-binding fragment thereof, optionally wherein the at least one monoclonal antibody, or antigen-binding fragment thereof is according to claim 16; and
c) comparing said level of the at least one ganglioside to that in a control sample,
wherein a significantly higher level of the at least one ganglioside in the subject sample as compared to the level in the control sample indicates a recurrence of a cancer in the subject.

52. A method of detecting a minimal residual disease in a subject, the method comprising:
a) obtaining or providing a sample from the subject in remission;
b) determining the level of at least one ganglioside in the subject sample using at least one monoclonal antibody, or antigen-binding fragment thereof, optionally wherein the at least one monoclonal antibody, or antigen-binding fragment thereof is according to claim 16; and
c) comparing said level of the at least one ganglioside to that in a control sample,
wherein a significantly higher level of the at least one ganglioside in the subject sample as compared to the level in the control sample indicates that the subject has a minimal residual disease.

53. A method of stratifying subjects afflicted with a cancer according to benefit from a cancer therapy, the method comprising:
a) determining the level of at least one ganglioside in a subject sample using at least one monoclonal antibody, or antigen-binding fragment thereof, optionally wherein the at least one monoclonal antibody, or antigen-binding fragment thereof is according to claim 16;
b) determining the level of the at least one ganglioside in a control; and
c) comparing the level of the at least one ganglioside detected in steps a) and b);
wherein no significant change or a decrease in the level of the at least one ganglioside in the subject sample as compared to the level in the control is an indication that the subject afflicted with the cancer would benefit from the cancer therapy.

54. A method of determining whether a subject afflicted with a cancer would likely respond to a cancer therapy, the method comprising:
a) determining the level of at least one ganglioside in a subject sample using at least one monoclonal antibody, or antigen-binding fragment thereof, optionally wherein the at least one monoclonal antibody, or antigen-binding fragment thereof is according to claim 16;
b) determining the level of the at least one ganglioside in a control; and
c) comparing the level of the at least one ganglioside detected in steps a) and b);
wherein a significantly higher level of the at least one ganglioside in the subject sample as compared to the level in the control is an indication that the subject afflicted with the cancer would not respond to the cancer therapy; and/or
wherein no significant change or a decrease in the level of the at least one ganglioside in the subject sample as compared to the level in the control is an indication that the subject afflicted with the cancer would respond to the cancer therapy.

55. A method for predicting the clinical outcome of a subject afflicted with a cancer, the method comprising:
a) determining the level of at least one ganglioside in a subject sample using at least one monoclonal antibody, or antigen-binding fragment thereof, optionally wherein the at least one monoclonal antibody, or antigen-binding fragment thereof is according to claim 16;
b) determining the level of the at least one ganglioside in a control; and
c) comparing the level of the at least one ganglioside determined in steps a) and b);
wherein a significantly higher level of the at least one ganglioside in the subject sample as compared to the level in the control is an indication that the subject has a poor clinical outcome.

56. A method of monitoring the progression of a cancer in a subject, the method comprising:
a) detecting in a subject sample at a first point in time the level of at least one ganglioside using at least one monoclonal antibody, or antigen-binding fragment thereof, optionally wherein the at least one monoclonal antibody, or antigen-binding fragment thereof is according to claim 16;
b) repeating step a) at a subsequent point in time; and
c) comparing the level of the at least one ganglioside detected in steps a) and b) to monitor the progression of the cancer in the subject, optionally wherein the subject is at risk for developing a cancer.

57. (canceled)

58. A method of assessing the efficacy of a cancer therapy in a subject, the method comprising:
a) determining the level of at least one ganglioside using at least one monoclonal antibody, or antigen-binding fragment thereof, optionally wherein the at least one monoclonal antibody, or antigen-binding fragment thereof is according to claim 16, in a first sample obtained from a subject;
b) repeating step a) during at least one subsequent point in time after administration of the cancer therapy; and
c) comparing the level of at least one ganglioside detected in steps a) and b),
wherein a significantly lower level of the at least one ganglioside in the at least one subsequent sample, relative to the first sample, is an indication that the therapy is efficacious to treat a cancer in the subject.

59-68. (canceled)

69. A method of diagnosing a cancer in a subject or a method of identifying a subject having a cancer, the method comprising:
a) determining the level of at least one ganglioside in a subject sample using mass spectrometry according to claim 45; and
b) comparing said level of the at least one ganglioside to that in a control sample,
wherein a significantly higher level of the at least one ganglioside in the subject sample as compared to the control sample indicates that the subject has a cancer.

70. (canceled)

71. A method of determining a grade of a cancer, the method comprising:
a) determining the level of at least one ganglioside in a subject sample using mass spectrometry according to claim 45; and
b) comparing said level of the at least one ganglioside to that in a control sample,
wherein at least 100% and no more than 200% increase in the level of the at least one ganglioside in the subject sample as compared to the level in the control sample indicates that the subject has low grade cancer; and/or
wherein at least 200% increase in the level of the at least one ganglioside in the subject sample as compared to the level in the control sample indicates that the subject has a high grade cancer.

72. A method of determining a tumor burden of a cancer, the method comprising:
a) determining the level of at least one ganglioside in a subject sample using mass spectrometry according to claim 45; and
b) comparing said level of the at least one ganglioside to that in a control sample,
wherein at least 100% and no more than 200% increase in the level of the at least one ganglioside in the subject sample as compared to the level in the control sample indicates that the subject has a low tumor burden; and/or
wherein at least 200% increase in the level of the at least one ganglioside in the subject sample as compared to the level in the control sample indicates that the subject has a high tumor burden.

73. A method of detecting a recurrence of a cancer in a subject, the method comprising:
a) obtaining or providing a sample from the subject whose cancer has regressed after receiving cancer treatment;
b) determining the level of at least one ganglioside in the subject sample using mass spectrometry according to claim 45; and
c) comparing said level of the at least one ganglioside to that in a control sample,
wherein a significantly higher level of the at least one ganglioside in the subject sample as compared to the level in the control sample indicates a recurrence of a cancer in the subject.

74. A method of detecting a minimal residual disease in a subject, the method comprising:
a) obtaining or providing a sample from the subject in remission;
b) determining the level of at least one ganglioside in the subject sample using mass spectrometry according to claim 45; and
c) comparing said level of the at least one ganglioside to that in a control sample,
wherein a significantly higher level of the at least one ganglioside in the subject sample as compared to the level in the control sample indicates that the subject has a minimal residual disease.

75. A method of stratifying subjects afflicted with a cancer according to benefit from a cancer therapy, the method comprising:
a) determining the level of at least one ganglioside in a subject sample using mass spectrometry according to claim 45;
b) determining the level of the at least one ganglioside in a control; and
c) comparing the level of the at least one ganglioside detected in steps a) and b);
wherein no significant change or a decrease in the level of the at least one ganglioside in the subject sample as compared to the level in the control is an indication that the subject afflicted with the cancer would benefit from the cancer therapy.

76. A method of determining whether a subject afflicted with a cancer would likely respond to a cancer therapy, the method comprising:
a) determining the level of at least one ganglioside in a subject sample using mass spectrometry according to claim 45;
b) determining the level of the at least one ganglioside in a control; and
c) comparing the level of the at least one ganglioside detected in steps a) and b);
wherein a significantly higher level of the at least one ganglioside in the subject sample as compared to the level in the control is an indication that the subject afflicted with the cancer would not respond to the cancer therapy; and/or
wherein no significant change or a decrease in the level of the at least one ganglioside in the subject sample as compared to the level in the control is an indication that the subject afflicted with the cancer would respond to the cancer therapy.

77. A method for predicting the clinical outcome of a subject afflicted with a cancer, the method comprising:
a) determining the level of at least one ganglioside in a subject sample using mass spectrometry according to claim 45;
b) determining the level of the at least one ganglioside in a control; and
c) comparing the level of the at least one ganglioside determined in steps a) and b);
wherein a significantly higher level of the at least one ganglioside in the subject sample as compared to the level in the control is an indication that the subject has a poor clinical outcome.

78. A method of monitoring the progression of a cancer in a subject, the method comprising:
a) detecting in a subject sample at a first point in time the level of at least one ganglioside using mass spectrometry according to claim 45;
b) repeating step a) at a subsequent point in time; and
c) comparing the level of the at least one ganglioside detected in steps a) and b) to monitor the progression of the cancer in the subject, optionally wherein the subject is at risk for developing a cancer.

79. (canceled)

80. A method of assessing the efficacy of a cancer therapy in a subject, the method comprising:
a) determining the level of at least one ganglioside using mass spectrometry according to claim 45, in a first sample obtained from a subject;
b) repeating step a) during at least one subsequent point in time after administration of the cancer therapy; and
c) comparing the level of at least one ganglioside detected in steps a) and b),
wherein a significantly lower level of the at least one ganglioside in the at least one subsequent sample, relative to the first sample, is an indication that the therapy is efficacious to treat a cancer in the subject.

81. (canceled)

82. A method of diagnosing a cancer in a subject or a method of identifying a subject having a cancer, the method comprising:
a) determining the lipid length of at least one ganglioside in a subject sample using mass spectrometry according to claim 45; and
b) comparing the said lipid length of the at least one ganglioside to that in a control sample,
wherein a significant change in heterogeneity of the lipid length of the at least one ganglioside in the subject sample as compared to the control sample indicates that the subject has a cancer.

83. (canceled)

84. A method of determining a grade of a cancer, the method comprising:
a) determining the lipid length of at least one ganglioside in the subject sample using mass spectrometry according to claim 45; and
b) comparing the said lipid length of the at least one ganglioside to that in a control sample,
wherein at least 100% and no more than 200% change (e.g., increase or decrease) in heterogeneity of the lipid length of the at least one ganglioside in the subject sample as compared to that in the control sample indicates that the subject has a low grade cancer; and/or
wherein at least 200% change (e.g., increase or decrease) in heterogeneity of the lipid length of the at least one ganglioside in the subject sample as compared to that in the control sample indicates that the subject has a high grade cancer.

85. A method of determining a tumor burden of a cancer, the method comprising:
a) determining the lipid length of at least one ganglioside in the subject sample using mass spectrometry according to claim 45; and
b) comparing the said lipid length of the at least one ganglioside to that in a control sample,
wherein at least 100% and no more than 200% change (e.g., increase or decrease) in heterogeneity of the lipid length of the at least one ganglioside in the subject sample as compared to that in the control sample indicates that the subject has a low tumor burden; and/or
wherein at least 200% change (e.g., increase or decrease) in heterogeneity of the lipid length of the at least one ganglioside in the subject sample as compared to that in the control sample indicates that the subject has a high tumor burden.

86. A method of detecting a recurrence of a cancer in a subject, the method comprising:
a) obtaining or providing a sample from the subject whose cancer has regressed after receiving cancer treatment;
b) determining the lipid length of at least one ganglioside in the subject sample using mass spectrometry according to claim 45; and
c) comparing the said lipid length of the at least one ganglioside to that in a control sample,
wherein a significant change in heterogeneity of the lipid length of the at least one ganglioside in the subject sample as compared to that in the control sample indicates a recurrence of a cancer in a subject.

87. A method of detecting a minimal residual disease in a subject, the method comprising:
a) obtaining or providing a sample from the subject in remission;
b) determining the lipid length of at least one ganglioside in the subject sample using mass spectrometry according to claim 45; and
c) comparing the said lipid length of the at least one ganglioside to that in a control sample,
wherein a significant change in heterogeneity of the lipid length of the at least one ganglioside in the subject sample as compared to that in the control sample indicates that the subject has a minimal residual disease.

88. A method of stratifying subjects afflicted with a cancer according to benefit from a cancer therapy (e.g., immunotherapy), the method comprising:
a) determining the lipid length of at least one ganglioside in a subject sample using mass spectrometry according to claim 45;
b) determining the lipid length of the at least one ganglioside in a control; and
c) comparing the the lipid length of the at least one ganglioside detected in steps a) and b);
wherein no significant change in heterogeneity of the lipid length of the at least one ganglioside in the subject sample as compared to that in the control is an indication that the subject afflicted with the cancer would benefit from the cancer therapy.

89. A method of determining whether a subject afflicted with a cancer would likely respond to a cancer therapy (e.g., immunotherapy), the method comprising:
a) determining the lipid length of at least one ganglioside in a subject sample using mass spectrometry according to claim 45;
b) determining the lipid length of the at least one ganglioside in a control; and
c) comparing the lipid length of the at least one ganglioside detected in steps a) and b);
wherein a significant change in heterogeneity of the lipid length of the at least one ganglioside in the subject sample as compared to that in the control is an indication that the subject afflicted with the cancer would not respond to the cancer therapy; and/or
wherein no significant change in heterogeneity of the lipid length of the at least one ganglioside in the subject sample as compared to that in the control is an indication that the subject afflicted with the cancer would respond to the cancer therapy.

90. A method for predicting the clinical outcome of a subject afflicted with a cancer, the method comprising:
a) determining the lipid length of at least one ganglioside in a subject sample using mass spectrometry according to claim 45;
b) determining the lipid length of the at least one ganglioside in a control; and
c) comparing the lipid length of the at least one ganglioside determined in steps a) and b);
wherein a significant change in heterogeneity of the lipid length of the at least one ganglioside in the subject sample as compared to the control sample is an indication that the subject has a poor clinical outcome.

91. A method of monitoring the progression of a cancer in a subject, the method comprising:
a) detecting in a subject sample at a first point in time the lipid length of at least one ganglioside using mass spectrometry according to claim 45;
b) repeating step a) at a subsequent point in time; and
c) comparing heterogeneity of the lipid length of the at least one ganglioside detected in steps a) and b) to monitor the progression of the cancer in the subject, optionally wherein the subject is at risk for developing a cancer.

92. (canceled)

93. A method of assessing the efficacy of a cancer therapy in a subject, the method comprising:
a) determining the lipid length of at least one ganglioside using mass spectrometry according to claim 45, in a first sample obtained from the subject;
b) repeating step a) during at least one subsequent point in time after administration of the cancer therapy; and
wherein a significant change in heterogeneity of the lipid length of the at least one ganglioside in the second sample, relative to the first sample, is an indication that the therapy is efficacious to treat a cancer in the subject.

94-110. (canceled)
